SEQUENCE LISTING (42 sequences)

SEQ ID NO: 1 - DNA, 141 bp, Artificial Sequence
  TCAG-MS2dgRNA
gtttcagagctaggccagcatgaggatcacccatgcctgcagggcctagcaagttgaaataaggctagtccgttatcaacttggccagcatgaggatcacccatgcctgcagggccaagtggcaccgagtcggtgcttttt

SEQ ID NO: 2 - DNA, 145 bp, Artificial Sequence
  TC5GC-MS2dgRNA
gtttcagagctagggccaacatgaggatcacccatgtctgcagggccctagcaagttgaaataaggctagtccgttatcaacttgggccaacatgaggatcacccatgtctgcagggcccaagtggcaccgagtcggtgcttttt

SEQ ID NO: 3 - DNA, 141 bp, Artificial Sequence
  TC-MS2dgRNA
gtttcagagctaggccaacatgaggatcacccatgtctgcagggcctagcaagttgaaataaggctagtccgttatcaacttggccaacatgaggatcacccatgtctgcagggccaagtggcaccgagtcggtgcttttt

SEQ ID NO: 4 - DNA, 145 bp, Artificial Sequence
  5GC-MS2dgRNA
gttttagagctagggccaacatgaggatcacccatgtctgcagggccctagcaagttaaaataaggctagtccgttatcaacttgggccaacatgaggatcacccatgtctgcagggcccaagtggcaccgagtcggtgcttttt

SEQ ID NO: 5 - DNA, 141 bp, Artificial Sequence
  MS2gRNA
gttttagagctaggccaacatgaggatcacccatgtctgcagggcctagcaagttaaaataaggctagtccgttatcaacttggccaacatgaggatcacccatgtctgcagggccaagtggcaccgagtcggtgcttttt

SEQ ID NO: 6 - DNA, 146 bp, Artificial Sequence
  SE-MS2gRNA
gttttagagctatgctgaacatgaggatcacccatgtctgcagcagcatagcaagttaaaataaggctagtccgttatcaactttgctgaacatgaggatcacccatgtctgcagcagcaaagtggcaccgagtcggtgctttttt

SEQ ID NO: 7 - DNA, 12 bp, Artificial Sequence
  native backbone
gttttagagcta

SEQ ID NO: 8 - DNA, 36 bp, Artificial Sequence
  native backbone
tagcaagttaaaataaggctagtccgttatcaactt

SEQ ID NO: 9 - DNA, 22 bp, Artificial Sequence
  native backbone
aagtggcaccgagtcggtgctt

SEQ ID NO: 10 - DNA, 12 bp, Artificial Sequence
  modified backbone
gtttcagagcta

SEQ ID NO: 11 - DNA, 36 bp, Artificial Sequence
  modified backbone
tagcaagttgaaataaggctagtccgttatcaactt

SEQ ID NO: 12 - DNA, 34 bp, Artificial Sequence
  native MS2 binding loop
ggccaacatgaggatcacccatgtctgcagggcc

SEQ ID NO: 13 - DNA, 36 bp, Artificial Sequence
  modified MS2 binding loop
tgctgaacatgaggatcacccatgtctgcagcagca

SEQ ID NO: 14 - DNA, 36 bp, Artificial Sequence
  modified MS2 binding loop
gggccaacatgaggatcacccatgtctgcagggccc

SEQ ID NO: 15 - DNA, 34 bp, Artificial Sequence
  modified MS2 binding loop
ggccagcatgaggatcacccatgcctgcagggcc

SEQ ID NO: 16 - protein, 1368 aa, Artificial Sequence
  Cas9
MDKKYSIGLDIGTNSVGWAVITDDYKVPSKKFKVLGNTDRHSIKKNLIGALLFDSGEIAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMLAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPTAFKYFDTTIDRKRYTSTKEVLDATFIHQSITGLYETRIDLSQLGGD

SEQ ID NO: 17 - protein, 1367 aa, Artificial Sequence
  dCas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGG

SEQ ID NO: 18 - protein, 473 aa, Artificial Sequence
  MH2-p65-HSF1
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 19 - DNA, 54 bp, Artificial Sequence
  primer with deep sequencing adaptor
acactctttccctacacgacgctcttccgatctcatggtttagaagagggagga

SEQ ID NO: 20 - DNA, 51 bp, Artificial Sequence
  primer with deep sequencing adaptor
gactggagttcagacgtgtgctcttccgatctgagcaggcagcatagcagt

SEQ ID NO: 21 - DNA, 158 bp, Artificial Sequence
  20bp-MS2gRNA
  misc_feature (1)..(20): n is a, c, g, t or u
nnnnnnnnnnnnnnnnnnnnguuuuagagcuaggccaacaugaggaucacccaugucugcagggccuagcaaguuaaaauaaggcuaguccguuaucaacuuggccaacaugaggaucacccaugucugcagggccaaguggcaccgagucggugcuu

SEQ ID NO: 22 - DNA, 152 bp, Artificial Sequence
  14bp-MS2gRNA
  misc_feature (1)..(14): n is a, c, g, t or u
nnnnnnnnnnnnnnguuuuagagcuaggccaacaugaggaucacccaugucugcagggccuagcaaguuaaaauaaggcuaguccguuaucaacuuggccaacaugaggaucacccaugucugcagggccaaguggcaccgagucggugcuu

SEQ ID NO: 23 - DNA, 152 bp, Artificial Sequence
  14bp-TCAG-MS2dgRNA
  misc_feature (1)..(14): n is a, c, g, t or u
nnnnnnnnnnnnnnguuucagagcuaggccagcaugaggaucacccaugccugcagggccuagcaaguugaaauaaggcuaguccguuaucaacuuggccagcaugaggaucacccaugccugcagggccaaguggcaccgagucggugcuu

SEQ ID NO: 24 - DNA, 156 bp, Artificial Sequence
  14bp-SE-MS2dgRNA
  misc_feature (1)..(14): n is a, c, g, t or u
nnnnnnnnnnnnnnguuuuagagcuaugcugaacaugaggaucacccaugucugcagcagcauagcaaguuaaaauaaggcuaguccguuaucaacuuugcugaacaugaggaucacccaugucugcagcagcaaaguggcaccgagucggugcuu

SEQ ID NO: 25 - DNA, 152 bp, Artificial Sequence
  14bp-TC-MS2dgRNA
  misc_feature (1)..(14): n is a, c, g, t or u
nnnnnnnnnnnnnnguuucagagcuaggccaacaugaggaucacccaugucugcagggccuagcaaguugaaauaaggcuaguccguuaucaacuuggccaacaugaggaucacccaugucugcagggccaaguggcaccgagucggugcuu

SEQ ID NO: 26 - DNA, 156 bp, Artificial Sequence
  14bp-5GC-MS2dgRNA
  misc_feature (1)..(14): n is a, c, g, t or u
nnnnnnnnnnnnnnguuuuagagcuagggccaacaugaggaucacccaugucugcagggcccuagcaaguuaaaauaaggcuaguccguuaucaacuugggccaacaugaggaucacccaugucugcagggcccaaguggcaccgagucggugcuu

SEQ ID NO: 27 - DNA, 156 bp, Artificial Sequence
  14bp-TC5GC-MS2dgRNA
  misc_feature (1)..(14): n is a, c, g, t or u
nnnnnnnnnnnnnnguuucagagcuagggccaacaugaggaucacccaugucugcagggcccuagcaaguugaaauaaggcuaguccguuaucaacuugggccaacaugaggaucacccaugucugcagggcccaaguggcaccgagucggugcuu

SEQ ID NO: 28 - DNA, 141 bp, Artificial Sequence
  TCAG-MS2dgRNA
guuucagagcuaggccagcaugaggaucacccaugccugcagggccuagcaaguugaaauaaggcuaguccguuaucaacuuggccagcaugaggaucacccaugccugcagggccaaguggcaccgagucggugcuuuuu

SEQ ID NO: 29 - DNA, 145 bp, Artificial Sequence
  TC5GC-MS2dgRNA
guuucagagcuagggccaacaugaggaucacccaugucugcagggcccuagcaaguugaaauaaggcuaguccguuaucaacuugggccaacaugaggaucacccaugucugcagggcccaaguggcaccgagucggugcuuuuu

SEQ ID NO: 30 - DNA, 141 bp, Artificial Sequence
  TC-MS2dgRNA
guuucagagcuaggccaacaugaggaucacccaugucugcagggccuagcaaguugaaauaaggcuaguccguuaucaacuuggccaacaugaggaucacccaugucugcagggccaaguggcaccgagucggugcuuuuu

SEQ ID NO: 31 - DNA, 145 bp, Artificial Sequence
  5GC-MS2dgRNA
guuuuagagcuagggccaacaugaggaucacccaugucugcagggcccuagcaaguuaaaauaaggcuaguccguuaucaacuugggccaacaugaggaucacccaugucugcagggcccaaguggcaccgagucggugcuuuuu

SEQ ID NO: 32 - DNA, 141 bp, Artificial Sequence
  MS2gRNA
guuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuuuu

SEQ ID NO: 33 - DNA, 146 bp, Artificial Sequence
  SE-MS2gRNA
guuuuagagcuaugcugaacaugaggaucacccaugucugcagcagcauagcaaguuaaaauaaggcuaguccguuaucaacuuugcugaacaugaggaucacccaugucugcagcagcaaaguggcaccgagucggugcuuuuuu

SEQ ID NO: 34 - DNA, 12 bp, Artificial Sequence
  native backbone
guuuuagagcua

SEQ ID NO: 35 - DNA, 36 bp, Artificial Sequence
  native backbone
uagcaaguuaaaauaaggcuaguccguuaucaacuu

SEQ ID NO: 36 - DNA, 22 bp, Artificial Sequence
  native backbone
aaguggcaccgagucggugcuu

SEQ ID NO: 37 - DNA, 12 bp, Artificial Sequence
  modified backbone
guuucagagcua

SEQ ID NO: 38 - DNA, 36 bp, Artificial Sequence
  modified backbone
uagcaaguugaaauaaggcuaguccguuaucaacuu

SEQ ID NO: 39 - DNA, 34 bp, Artificial Sequence
  native MS2 binding loop
ggccaacaugaggaucacccaugucugcagggcc

SEQ ID NO: 40 - DNA, 36 bp, Artificial Sequence
  modified MS2 binding loop
ugcugaacaugaggaucacccaugucugcagcagca

SEQ ID NO: 41 - DNA, 36 bp, Artificial Sequence
  modified MS2 binding loop
gggccaacaugaggaucacccaugucugcagggccc

SEQ ID NO: 42 - DNA, 34 bp, Artificial Sequence
  modified MS2 binding loop
ggccagcaugaggaucacccaugccugcagggcc

(19) United States
(12) Patent Application Publication
(10) Pub. No.: US 2021/0102206 A1
(43) Pub. Date: Apr. 8, 2021

(54) TARGETED GENE ACTIVATION USING MODIFIED GUIDE RNA

(71) Applicant: Salk Institute for Biological Studies, La Jolla, CA (US)

(72) Inventors: Hsin-Kai Liao, San Diego, CA (US); Fumiyuki Hatanaka, San Diego, CA (US); Juan Carlos Izpisua Belmonte, La Jolla, CA (US)

(73) Assignee: Salk Institute for Biological Studies, La Jolla, CA (US)

(21) Appl. No.: 17/104,372

(22) Filed: Nov. 25, 2020

Related U.S. Application Data
(63) Continuation of application No. PCT/US2018/036350, filed on Jun. 6, 2018.

(51) Int. Cl. C12N 15/113; C12N 15/67; C12N 15/86; C12N 9/22; C07K 14/08

(52) U.S. Cl. CPC C12N 15/113; C12N 15/67; C12N 15/86; C12N 2310/531; C07K 14/08; C12N 2750/14143; C12N 9/22

(57) ABSTRACT

Provided are modified guide RNAs (gRNAs), including dead guide RNAs (dgRNAs) with increased GC content and/or decreased repetitive content, as well as compositions and kits including such dgRNAs, which can be used in a targeted gene activation system, for example to increase expression of a gene to treat a disease in vivo. Such methods increase targeted gene expression, without creating DNA double strand breaks (DSBs).

### CROSS REFERENCE TO RELATED APPLICATION

This application is a continuation of International Application No. PCT/US2018/036350 filed Jun. 6, 2018, which was published in English under PCT Article 21(2), herein incorporated by reference in its entirety.

### ACKNOWLEDGMENT OF GOVERNMENT SUPPORT

This invention was made with government support under HL123755 awarded by The National Institutes of Health. The government has certain rights in the invention.

### FIELD

This application provides modified guide RNAs (gRNAs), including dead guide RNAs (dgRNAs) with increased GC and shortened repetitive content, as well as compositions and kits including such dgRNAs, which can be used in a targeted gene activation system, for example, to increase expression of a gene to reprogram a cell or to treat a disease in vivo.

### BACKGROUND

Over the past 30 years, epigenetic therapies have been developed to treat many human diseases, including cancer, diabetes, autoimmunity, and genetic disorders (Heerboth et al., 2014; Pfister and Ashworth, 2017). Most of these approaches have relied on drugs (‘epi-drugs’) that ubiquitously alter epigenetic marks (e.g., DNA methylation or histone modifications). However, these epi-drugs are not without risk, as off-target genes may be affected (Altucci and Rots, 2016; Hunter, 2015). Therefore, new methods for generating targeted epigenetic modifications to alter the expression of specific genes is desired (de Groote et al., 2012; Jurkowski et al., 2015; Takahashi et al., 2017).

Advances in genome editing technologies have revolutionized a wide range of scientific fields, from basic sciences to translational medicine. In particular, discovery of the bacterial immune system CRISPR/Cas9 has led to the development of tools for rapid and efficient RNA-based, sequence-specific genome editing (Jinek et al., 2012). In addition to enabling the engineering of eukaryotic genomes, recent alterations to the CRISPR/Cas9 system have provided opportunities for regulating gene expression and for creating epigenetic alterations without introducing DNA double-strand breaks (DSBs) (Qi et al., 2013), which can avoid creating undesired permanent mutations in target genomes. Original efforts to convert the CRISPR/Cas9 gene editing system into a transactivator were achieved by fusing a transcriptional activation domain (VP64) to versions of Cas9 that lacked nuclease activity (dCas9) (Gilbert et al., 2013; Perez-Pinera et al., 2013), which enabled the CRISPR/Cas9 system to transcriptionally activate target genes within the native chromosomal context. This transformative technology can provide the foundation for many scientific and medical applications, including: 1) performing functional genetic screens, 2) creating synthetic gene circuits, 3) developing therapeutic interventions to compensate for genetic defects, and 4) redirecting cell fate by epigenetic reprogramming for regenerative medicine (Chen and Qi, 2017; Thakore et al., 2016; Vora et al., 2016).

The original version of the dCas9-VP64 system cannot stimulate robust target gene activation (TGA) using a single guide RNA (sgRNA). In most cases, TGA efficiency relies on recruitment of multiple sgRNAs to the target gene (Gilbert et al., 2013; Kearns et al., 2014), which diminishes the utility of this epigenetic tool. To improve the efficiency of CRISPR/Cas9-mediated TGA, multiple transcriptional activation domains were fused or recruited to the dCas9/gRNA complex (e.g., the tripartite activator system (dCas9-VPR), synergistic activation mediator (SAM), or dCas9-Suntag) (Chavez et al., 2015; Konermann et al., 2015; Tanenbaum et al., 2014). These second-generation CRISPR/Cas9 TGA systems are effective for functional genetic studies by single gRNAs in vitro, but not in vivo (Komor et al., 2017; Thakore et al., 2016), primarily due to insufficient transduction of the Cas9 fusion protein in vivo and low levels of in vivo TGA. In addition, sequences encoding the dCas9/gRNA and co-transcriptional activator complexes exceed the capacity of most common viral vectors (e.g., adeno-associated virus (AAV)), which represent the most promising method for gene delivery in vivo (Komor et al., 2017; Thakore et al., 2016).

Thus, all previous CRISPR/Cas9-mediated epigenetic editing systems do not induce a physiologically relevant phenotype in a postnatal mammal (Jurkowski et al., 2015; Vora et al., 2016), which limits the utility of these tools for performing experiments and developing targeted epigenetic therapies.

### SUMMARY

Provided here are guide ribonucleic acid (gRNA) molecules, such as “dead” gRNA molecules (dgRNA), and methods of their use to activate transcription of one or more targets, such as a gene whose expression is reduced or eliminated resulting in disease. Use of the disclosed gRNA molecules and targeted gene activation (TGA) systems increase gene expression without inducing DNA double strand breads. In such methods, at least one CRISPR component (gRNA or Cas9) used is an inactivated (dead) form (e.g., a dead Cas9, a dead gRNA that includes only about 14 or 15 bp of complementary target sequence, or both).

In one example, a gRNA includes the structure A-B-C-D-E, wherein A is the 5′-end, and E is the 3′-end. For example, the gRNA can include a first region (e.g., A, in A-B-C-D-E) that includes a tetraloop backbone sequence that has at least 90%, at least 95%, at least 98%, at least 99%, or 100% sequence identity to the sequence gttttagagcta (SEQ ID NO: 7) or guuuuagagcua (SEQ ID NO: 34). The second region (e.g., B, in A-B-C-D-E) is linked to the first and third region (e.g., is in between the two regions A and C), and includes a modified MS2-binding loop sequence. The third region (e.g., C, in A-B-C-D-E) is linked to the second and fourth region (e.g., is in between the two regions B and D) and includes a stem-loop 1 and stem-loop 2 backbone sequence comprising at least 90%, at least 95%, at least 98%, at least 99%, or 100% sequence identity to the sequence tagcaagttaaaataaggctagtccgttatcaactt (SEQ ID NO: 8) or uagcaaguuaaaauaaggcuaguccguuaucaacuu (SEQ ID NO: 35). The fourth region (e.g., D, in A-B-C-D-E) linked to the third and fifth regions (e.g., is in between the two regions C and E) and includes the modified MS2-binding loop sequence. The fifth region (e.g., E, in A-B-C-D-E) is at the 3′-end of the gRNA, is linked to the fourth region, and includes a stem-loop 3 backbone sequence including at least 90%, at least 95%, at least 98%, at least 99%, or 100% sequence identity to aagtggcaccgagtcggtgctt (SEQ ID NO: 9) or aaguggcaccgagucggugcuu (SEQ ID NO: 36). The modified MS2-binding loop sequences of the gRNA include at least two nucleotide changes to the native MS2-binding loop sequence ggccaacatgaggatcacccatgtctgcagggcc (SEQ ID NO: 12) or ggccaacaugaggaucacccaugucugcagggcc (SEQ ID NO: 39) that increase the GC content and/or shorten repetitive content of the modified MS2-binding loop sequence relative to the native MS2-binding loop sequence.

In another example, a gRNA can include a first region at the 5′-end (e.g., A, in A-B-C-D-E), which includes a first modified backbone sequence having at least one nucleotide change to the native tetraloop backbone sequence gttttagagcta (SEQ ID NO: 7) or guuuuagagcua (SEQ ID NO: 34). The second region (e.g., B, in A-B-C-D-E) is linked to the first region and to a third region (e.g., is in between region A and region C) and includes an MS2-binding loop sequence (such as ggccaacatgaggatcacccatgtctgcagggcc; SEQ ID NO: 12 or ggccaacaugaggaucacccaugucugcagggcc; SEQ ID NO: 39). The third region (e.g., C, in A-B-C-D-E) is linked to the second and fourth region (e.g., is in between the two regions B and D) and includes a second modified backbone sequence having at least one ribonucleotide change to the native stem-loop 1 and stem-loop 2 backbone sequence tagcaagttaaaataaggctagtccgttatcaactt (SEQ ID NO: 8) or uagcaaguuaaaauaaggcuaguccguuaucaacuu (SEQ ID NO: 35). The fourth region (e.g., D, in A-B-C-D-E) linked to the third and fifth regions (e.g., is in between region C and region E), and includes the MS2-binding loop sequence (such as ggccaacatgaggatcacccatgtctgcagggcc; SEQ ID NO: 12 or ggccaacaugaggaucacccaugucugcagggcc; SEQ ID NO: 39). The fifth region (e.g., E, in A-B-C-D-E) is at the 3′-end of the gRNA, is linked to the fourth region, and includes a stem-loop 3 backbone sequence comprising at least 90% sequence identity to aagtggcaccgagtcggtgctt (SEQ ID NO: 9) or aaguggcaccgagucggugcuu (SEQ ID NO: 36). The at least one nucleotide change in the first and second backbone sequence increases the GC content and/or shorten repetitive content of the first and second modified backbone sequences relative to the native backbone sequences.

In another example, the gRNA includes the structure T-A-B-C-D-E, wherein T is the 5′-end and E is the 3′-end. Thus, the gRNAs provided herein can include a sixth region at the 5′-end of the gRNA, which is linked at its 3′-end to the 5′ end of the first region of the gRNA (e.g., A in T-A-B-C-D-E). The sixth region (e.g., T in T-A-B-C-D-E) includes sufficient complementarity to a target nucleic acid molecule to hybridize to the target, and is about 14 to 30 nucleotides (or ribonucleotides) in length, such as at least about 14, 15, 16, 17, 18, 19, 20, 21, 22, 23, 24, 25, 26, 27, 28, 29, or 30 nucleotides (or ribonucleotides) in length. In some examples, the gRNA is a dead gRNA, wherein the sixth region (e.g., T in T-B-C-D-E) is about 14 or 15 nucleotides (or ribonucleotides) in length.

Also provided are compositions that include one or more gRNAs provided herein. Such compositions can further include a pharmaceutically acceptable carrier, such as water or saline.

Also provided are vectors that include one or more gRNAs provided herein, such as a viral vector, such as an AAV vector, such as an AAV9 vector.

Also provided are kits that include one or more gRNAs provided herein (which may be part of a vector, such as an AAV vector). The kits can further include a nucleic acid encoding a Cas9 protein or dead Cas9 (dCas9) protein (which may be part of a vector, such as an AAV vector). In some examples, the kits further include a Cas9 protein or dCas9 protein. The kits can further include a nucleic acid encoding an MS2-transcriptional activator fusion protein (such as MS2-p65-HSF1), which may be part of a vector (such as an AAV vector). In some examples, the nucleic acid encoding a Cas9 protein or dCas9 protein, and the nucleic acid encoding an MS2-transcriptional activator fusion protein are part of a single viral vector (e.g., AAV).

Also provided is a targeted gene activation (TGA) system. The system can include a first vector (such as a viral vector, e.g., AAV) that includes a nucleic acid encoding a Cas9 or dCas9 and a second vector (such as a viral vector, e.g., AAV) that includes a gRNA disclosed herein and a nucleic acid encoding an MS2-transcriptional activator fusion protein (such as MS2-p65-HSF1).

Methods of using the disclosed gRNAs and TGA systems are also provided. Such methods can be used to increase expression of at least one target gene product in a subject, such as a gene whose expression is decreased in the subject. In some examples, such methods treat a disease in the subject caused by the decreased expression of the target. In some examples, the methods increase expression of the target gene or gene product by at least 10%, at least 20%, at least 25%, at least 50%, at least 60%, at least 70%, at least 75%, at least 80%, at least 90%, at least 95%, at least 100%, at least 200%, at least 300%, at least 400%, at least 500%. Such methods include administering a therapeutically effective amount of a targeted gene activation (TGA) system to a subject, wherein the TGA system includes a first vector (such as a viral vector, e.g., AAV) that includes a nucleic acid molecule encoding a Cas9 protein or dCas9 protein and second vector (such as a viral vector, e.g., AAV) that includes one or more disclosed gRNAs, and a nucleic acid encoding an MS2-transcriptional activator fusion protein (such as MS2-p65-HSF1). Administration of the TGA system results in the first and second vectors infecting a cell of the subject, thereby increasing expression of the at least target one gene or gene product in the infected cell. Exemplary gene targets include Fst, Pdx1, klotho, utrophin, interleukin 10, and Six2.

Vector systems and kits for measuring gene activation when Cas9 (e.g., dCas9) is expressed are described herein. In some examples, the systems and kits include at least one gene activation vector and at least one reporter vector. In some examples, the at least one gene activation vector includes a guide ribonucleic acid (gRNA) and at least one transcriptional activator protein. In some examples, the at least one reporter vector includes a target sequence of the gRNA and at least one reporter protein, wherein the reporter protein is positioned downstream of the target sequence.

Methods of measuring gene activation in a subject (e.g., a mammal, such as a human or mouse) are also provided. In some examples, the methods can include expressing Cas9 (e.g., dCas9) in the subject. In some examples, the methods include injecting the subject with at least one gene activation vector and at least one reporter vector. In some examples, the at least one gene activation vector includes a guide ribonucleic acid (gRNA) and at least one transcriptional activator protein. In some examples, the at least one reporter vector includes a target sequence of the gRNA and at least one reporter protein, wherein the reporter protein is positioned downstream of the target sequence.

In some examples, the vector of the at least one gene activation vector or the at least one reporter vector is a viral vector (e.g., an AAV vector). In some examples, the gRNA includes a gRNA or dgRNA described herein. In some examples, the at least one transcriptional protein includes VP64, p65, MyoD1, HSF1, RTA, SET7/9, or any combination thereof. In specific examples, the at least one transcriptional protein includes P65 and HSF1. In some examples, the at least one reporter protein includes a fluorescent or bioluminescent protein (e.g., luciferase, mCherry, dTomato, or any combination thereof).

The foregoing and other objects and features of the disclosure will become more apparent from the following detailed description, which proceeds with reference to the accompanying figures.

### BRIEF DESCRIPTION OF THE DRAWINGS

FIGS. 1A-1G show a modified dgRNA-mediated CRISPR/Cas9 for target gene activation (CRISPR/Cas9 TGA). FIG. 1A shows a schematic representation of how sgRNAs, which include a truncated 14-bp gRNA (dgRNA) and MS2 loops, are introduced with the MS2-P65-HSF1 (MPH) transcriptional activation complex into Cas9-expressing mice for TGA. FIG. 1B shows that the luciferase reporter (tLuc) includes a dgRNA binding site (Target seq) followed by a minimal promoter (Pmin), a luciferase expression cassette (Luc), and a polyA termination signal. FIG. 1C shows modified gRNAs. MS2gRNA (or gRNA 2.0; SEQ ID NO: 21) includes a wild type 20-bp gRNA and stem-loops for MPH binding. MS2dgRNA (or dead gRNA; SEQ ID NO: 22) includes a truncated 14-bp MS2gRNA that can recruit MPH to activate gene expression without inducing Cas9-mediated double-stranded breaks. The MS2dgRNA (designated dgRNA; SEQ ID NO: 23) includes a 14-bp MS2dgRNA with modifications that enhance TGA. FIG. 1D shows that dgRNA modification improves the TGA of the tLuc reporter in 293 cells (left) and N2a cells that stably express Cas9 (right) (n=5 each). FIG. 1E shows that dgRNA modification results in Cas9/MPH-mediated TGA similar to the dCas9VP64/MPH/MS2gRNA SAM system in 293 cells (n=3). Without being bound by theory, TGA is weaker using wild-type gRNA likely due to DNA disruption. The data are means±SD. FIG. 1F shows administration of the CRISPR/Cas9 TGA system in vivo. tLuc and dgLuc/MPH (or gLuc/MPH) plasmids were injected (IM) into Cas9 mice followed by electroporation. FIG. 1G shows in vivo imaging (day 9 post-IM injection), which reveals luciferase activity associated with the dgLuc/MPH construct, but not the gLuc/MPH control.

FIGS. 2A-2J show in vivo CRISPR/Cas9-mediated targeted gene activation of reporters in different organs of Cas9 mice. FIG. 2A shows AAV-tLuc-mCherry and AAV-dgLuc-CAG-MPH vectors. FIG. 2B shows in vivo TGA of Luc reporter in Cas9 mice by IM injection at P2.5. FIG. 2C shows luciferase imaging of Cas9 mice at P15 after IM injection of AAV-tLuc-mCherry and AAV-dgMock-MPH (dgMock) (left) or AAV-dgLuc-MPH (dgLuc) (right). FIG. 2D shows in vivo TGA of Luc reporter in Cas9 mice by intra-cerebral injection at P0.5. FIG. 2E shows luciferase imaging of Cas9 mice at P21 after intra-cerebral injection of AAV-tLuc-mCherry and AAV-dgMock-MPH (left) or AAV-dgLuc-MPH (right). FIG. 2F shows in vivo TGA of reporter in neonatal Cas9 mice by facial vein injection at P0.5. FIG. 2G shows luciferase imaging of Cas9 mice at P21 after facial vein injections of AAV-tLuc-mCherry and AAV-dgMock-MPH (middle) or AAV-dgLuc-MPH (right). A non-injected mouse (Ctrl) is shown on the left. FIG. 2H shows ex vivo luciferase imaging of the eye (Ey), brain (Br), pituitary gland (Pi), tongue (To), heart (He), lung (Lu), thymus (Th), liver (Li), spleen (Sp), pancreas (Pa), kidney (Ki), testis (Te), muscle (Mu), spinal cord (SC), stomach (St), small intestine (In), and cecum (Ce) 28 days following facial vein injection. The luciferase signal is primarily in the liver and heart (upper). Imaging the same tissues with a longer exposure time (lower) revealed lower levels of luciferase signal in Lu, Ki, Mu, and SC (Li and He were removed). FIG. 2I shows in vivo TGA of a reporter in 11-week-old adult Cas9 mice through tail vein injection. FIG. 2J shows luciferase imaging of Cas9 mice at 4, 6, 8, 10, 14, 21, and 28 days after tail vein injection of AAV-tLuc-mCherry and AAV-dgMock-MPH (middle) or AAV-dgLuc-MPH (right). Non-injected controls are shown (left).

FIGS. 3A-3J show enhancement of skeletal muscle mass by Cas9/dgRNA-MPH mediated follistatin overexpression. FIG. 3A shows the mouse follistatin (Fst) gene. dgRNA targets are indicated (arrows). Cas9-expressing N2a cells were transfected with indicated dgRNAs and MPH. Levels of Fst expression were analyzed using qRT-PCR 3 days after transfections (n=3). FIG. 3B shows in vivo TGA in neonatal Cas9 mice via IM injection of AAV-dgFst-T2-MPH (dgFst) or AAV-dgMock-MPH (dgMock) into hind limbs bilaterally at P2.5. FIG. 3C shows that Cas9 mice received IM injections of AAV-dgFst-T2-MPH at P2.5 and a qRT-PCR analysis of hindlimb muscle at P21 or 3 months for Fst induction. Gene expression fold-changes were quantified relative to AAV-dgMock-MPH controls (n=3). FIGS. 3D and 3E show that gross hindlimb muscle mass increased in Cas9 mice injected with dgFst at P45 (FIG. 3D) or 3 months (FIG. 3E). FIG. 3F shows muscle to body weight ratios for tibialis anterior (TA) and quadriceps femoris (QF) for no treatment controls (n=6) or 3 months after IM injection of dgMock (n=12) or dgFst (n=10). FIG. 3G shows representative images of H&E-stained TA muscles dissected 3 months after dgMock or dgFst injections. Scale bar, 200 μm. The mage in upper-left corner indicates the position of TA dissection. FIG. 3H shows higher magnification of sections in FIG. 3G. Scale bar, 200 μm. FIG. 3I shows immunostaining for laminin in a TA muscle section. Scale bar, 100 μm. FIG. 3J shows a grip strength test for fore and hind limbs of 2- or 3-month-old Cas9 mice (2-month-old: n=3, no treatment; n=5, dgMock; n=4, dgFst. 3-month-old: n=6, no treatment; n=5, dgMock; n=4, dgFst). The data are means±SD. See also FIGS. 9A-9F.

FIGS. 4A-4G show that induction of IL-10 or Klotho expression via CRISPR/Cas9 TGA ameliorates acute kidney injury. FIG. 4A shows a schematic of AAV administration to Cas9 mice via tail-vein injection to prevent cisplatin-induced acute kidney injury (AKI). FIG. 4B shows qRT-PCR analyses of Il10 and klotho expression in liver tissues from mice injected with AAV-dgIL-10-MPH (n=3) or AAV-dgKlotho-MPH (n=3). Fold enrichments were calculated relative to dgMock controls. FIG. 4C shows that AAV-dgKlotho-MPH increased serum levels of Klotho protein relative to dgMock controls in cisplatin-treated mice (n=6). Data are means±SD. FIG. 4D shows that blood urea nitrogen (BUN) and serum creatinine (S-cre) levels in cisplatin-induced AKI mice were reduced by AAV-mediated IL-10 or Klotho overexpression. FIGS. 4E and 4F show histological sections from indicated mice that were subjected to H&E and PAS staining (FIG. 4E) and quantified pathological features (FIG. 4F) (approximately 10-15 slides analyzed for 3-4 mice per group). Scale bar, 50 μm. The data are means±SD. FIG. 4G shows survival curves of mice treated with the indicated AAV-dgRNAs for 8 days followed by a higher dose (17 g/Kg) cisplatin challenge (via intraperitoneal injection; n=8 for each group). See also FIGS. 10A-10K.

FIGS. 5A-5I show that in vivo epigenetic activation of Pdx1 in liver cells using the CRISPR/Cas9 TGA described herein promotes remodeling of epigenetic marks. FIG. 5A shows the mouse Pdx1 gene. gRNA targets are indicated (arrows). FIG. 5B shows Cas9 mESCs that were transfected with indicated gRNAs. Activation of Pdx1 was analyzed by qRT-PCR 4 days after transfections. FIG. 5C shows a qRT-PCR analysis of in vivo Pdx1 gene induction in the liver tissue of Cas9 mice that received a tail vein injection of AAV-dgPdx1-T2-MPH (13 days post-injection). Gene expression fold-change was quantified relative to AAV-dgMock-MPH controls. FIGS. 5D and 5E show a qRT-PCR analysis of in vivo liver samples after Pdx1 gene induction in FIG. 5C. FIG. 5D shows the fold-change in Ins1 and Ins2 after Pdx1 induction in liver. FIG. 5E shows the fold-change in Pcsk1 after Pdx1 induction in liver. The data are means±SD. FIG. 5F shows immunofluorescence analyses of PDX1 protein levels in liver tissue of mice injected with AAV-dgPdx1-T2-MPH or AAV-dgMock-MPH. Hepatocyte nuclear factor 3-beta (HNF3β) (red) and PDX1 (green) are shown. Scale bars, 50 μm. FIGS. 5G-5I show that the dgRNA-mediated TGA system remodels epigenetic marks at Pdx1 target loci in vivo. FIG. 5G shows a distribution of H3K4me3, H3K27ac, and CpG islands (green bars) at the Pdx1 locus in small intestine (In) and liver (Li) tissue (UCSC genome browser). Black bars are ChIP-qPCR regions, and the red bar is the dgRNA target. FIGS. 5H and 5I show ChIP-qPCR analyses for H3K4me3 (FIG. 5H), H3K27ac (FIG. 5I), and IgG (negative control) in the liver tissue of Cas9 mice that received tail vein injections of AAV-dgMock-MPH or AAV-dgPdx1-MPH. Relative real-time PCR values compared to the input are shown. The data are means±SEM. See also FIGS. 11A-11G.

FIGS. 6A-6F show that CRISPR/Cas9 TGA of utrophin rescues muscle phenotypes of dystrophin-deficient mice. FIG. 6A shows part of the mouse utrophin gene. dgRNA targets are indicated (arrows). FIG. 6B shows that Cas9 mESCs were transfected with indicated dgRNAs. Levels of Utrn activation were analyzed via qRT-PCR 3 days after transfections (n=3). FIG. 6C shows in vivo TGA in neonatal Cas9 or Cas9/mdx mice via IM injection of AAV-dgUtrn-T2-MPH (dgUtrn-T2), AAV-dgUtrn-T16-MPH (dgUtrn-T16), or AAV-dgMock-MPH (dgMock) at P2.5. FIG. 6D shows a qRT-PCR analysis of hind limb muscles of Cas9 mice 4 weeks after IM injections of AAV-dgUtrn at P2.5. Gene expression fold-changes were quantified relative to AAV-dgMock-MPH controls (n=3). FIG. 6E shows immunofluorescence of Utrophin in Cas9 mouse TA muscle injected with dgUtrn-T2, dgUtrn-T16, or dgMock (Utrophin, pink; DAPI, blue). Scale bars, 100 μm. FIG. 6F shows physiological analyses of mdx mice in Cas9 transgenic background (Cas9/mdx) injected with AAV-dgUtrn-T2 or AAV-dgMock into hind limb muscle at P2.5. A grip strength test was used for fore limbs and hind limbs of 2-month-old mice (n=6 (male) and 10 (female) for untreated Cas9 mice, n=9 (male) and 6 (female) for untreated Cas9/mdx mice, n=8 (male) and 9 (female) for AAV-dgMock injected Cas9/mdx mice, and n=7 (male) and 10 (female) for AAV-dgUtrn-T2 injected Cas9/mdx mice). The data are means±SD.

FIGS. 7A-7F show amelioration of dystrophic phenotypes of Mdx mice using a dual AAV-CRISPR/Cas9 TGA that includes AAV-Cas9 and AAV-dgRNA-MPH. FIG. 7A shows neonatal IM co-injection of AAV-SpCas9 and AAV-dgFst-T2-MPH (dgFst-T2) or AAV-dgUtrn-T2-MPH (dgUtrn-T2) to treat mdx mice. FIG. 7B shows that, three months post-IM injection, gross hindlimb muscle mass was increased in mdx mice co-injected with AAV-SpCas9 and AAV-dgFst-T2 compared with AAV-dgMock controls. FIG. 7C shows that TA muscle weight increased in male mdx mice (3 months old) co-injected (IM) with AAV-SpCas9 and dgFst-T2 compared with no treatment and dgMock controls (n=4 for each group). FIG. 7D shows a grip strength test for hind limbs of 2- and 3-month-old female mice (for 2-month-old mice: n=5 for non-injected wild type, n=10 for non-injected mdx mice, n=4 for AAV-SpCas9+dgMock, n=6 for AAV-SpCas9+dgFst-T2; for 3-month-old mice: n=11 for non-injected wild type, n=7 for non-injected mdx mice, n=3 for AAV-SpCas9+dgMock, and n=4 for AAV-SpCas9+dgFst-T2). FIG. 7E shows a grip strength test for fore and hind limbs of 2-month-old female mice. AAVs were injected into both fore and hind limbs bilaterally (n=5 for wild type mice, n=10 for non-injected mdx mice, n=15 for mdx mice co-injected with AAV-SpCas9+AAV-dgMock, n=12 for mdx mice co-injected with AAV-SpCas9+AAV-dgUtrn-T2). The data are means±SD. FIG. 7F shows a ChIP-qPCR analysis for H3K27ac and IgG (negative control) in muscle tissue of 3-month-old mdx mice that received IM co-injections of AAV-SpCas9 with either AAV-dgMock-MPH or AAV-dgFst-T2-MPH. Relative real-time qPCR values compared with the input are shown. The analytic regions for ChIP-qPCR are indicated at the bottom as shown in FIG. 14D. The data are means±SEM.

FIGS. 8A-8F show systematic improvement of the CRISPR/Cas9 gene activation related to FIG. 1. FIG. 8A shows sequences of the original and modified MS2gRNAs (SEQ ID NO: 22, SEQ ID NO: 24, SEQ ID NO: 25, SEQ ID NO: 26, SEQ ID NO: 27, and SEQ ID NO: 23, top to bottom, left to right). Target base pairing region (blue), MS2-binding stem-loops (green), and modified nucleotides (purple) are shown. FIG. 8B shows transcriptional activation of tLuc reporter in 293 cells using Cas9/MPH and different truncated gLucs (n=3). FIG. 8C shows modification of the MS2 transactivation complex. FIG. 8D shows transcriptional activation of tLuc reporter in embryonic stem cells derived from mice expressing active Cas9 (Cas9 mESCs) using dgLuc and different modified MS2-fused transactivators, as shown in FIG. 8C. The data are means±SD. FIGS. 8E and 8F show in vivo tLuc reporter induction in wild-type mice. FIG. 8E shows intramascular (IM) injections of components followed by electroporation to transduce gene activation. FIG. 8F shows in vivo imaging of luciferase signals 4 days post-IM injection of dCas9VP64/dgLuc/MPH or MPR constructs with tLuc reporters.

FIGS. 9A-9F show enhancement of skeletal muscle mass by Cas9/dgRNA-MPH-mediated Fst induction through facial vein injection related to FIGS. 3A-3J. FIG. 9A shows in vivo TGA in neonatal Cas9 mice via facial vein injections of dgFst or PBS at P0.5. FIG. 9B shows a qRT-PCR analysis of in vivo Fst gene induction in heart, liver, and muscle tissues dissected at P21. Gene expression fold changes were quantified relative to PBS controls (n=3). FIG. 9C shows representative images of H&E-stained tibialis anterior (TA) and quadriceps femoris (QF) muscles dissected 12 weeks after dgFst or PBS injections. Scale bar, 100 μm. FIG. 9D shows immunostaining of laminin in TA and QF muscles sections. Scale bar, 100 μm. FIG. 9E shows fiber size distributions of TA (upper) and QF (lower) muscles via H&E staining. FIG. 9F shows muscle to body weight ratios for TA and QF muscles 12 weeks after dgFst (n=11) or PBS (n=7) injections. The data are means±SD.

FIGS. 10A-10K show the transcriptional level of Klotho in mouse kidney and an evaluation of CRISPR/Cas9-mediated indel generation or gene activation both in vitro and in vivo related to FIGS. 4A-4G. FIG. 10A shows a qRT-PCR analysis of klotho transcript levels in kidney. Gene expression was quantified for aged kidneys (75-week-old), kidneys subjected to unilateral ureteral obstruction for 2 weeks (UUO, a model of chronic kidney disease), and kidneys treated with cisplatin for 4 days (a model of acute kidney disease) relative to 13-week-old healthy kidneys (n=2-4 mice per group). FIG. 10B shows establishment of a Cas9-expressing mouse embryonic stem cell line (Cas9 mESC) from Cas9 knockin mice for examining the activity of target gene induction by dgRNA-MPH with different target sites. FIG. 10C shows a schematic of the mouse Il10 and klotho genes. The arrows indicate target sites and directions of dgRNAs. Cas9 mESCs were transfected with the indicated dgRNAs. Levels of Il-10 and klotho activation were analyzed via qRT-PCR 3 days after transfections (n=3). FIG. 10D shows an evaluation of the specificity of CRISPR/Cas9-mediated TGA in vivo. The CRISPR/Cas9 TGA of the Il10 and klotho promoters were tested for off-target TGA using RNA-seq. Cas9 mice received tail vein injections of indicated AAV-dgRNA-MPHs, and RNA was extracted from livers of individual mice 13 days later. Expression levels were compared to a mouse injected with AAV-dgMock-MPH (dgMock). Il10 and klotho are indicated. Averages from n=3 replicates per group are shown. In FIG. 10E, the SURVEYOR® mutation detection assay was used to detect indel formation in cultured N2a cells that stably express wild-type Cas9. Cells were transduced with either regular 20-bp MS2gRNAs or truncated 14-bp MS2gRNAs. M, loading marker. In FIG. 10F, the SURVEYOR® mutation detection assay was used to detect indel formation in vivo. Mouse liver samples were collected from mice that received tail vein injections of AAV-gMock-MPH, AAV-gIl10-MPH, or AAV-dgIl10-MPH (13 days post-injection). dgRNAs with 14-bp of homology to target DNA did not induce detectable indel formation. The stars indicate the cleavage products of the samples indicated above each lane, and the numbers at the bottom indicate estimated indel frequency. FIGS. 10G and 10H show gene activation in N2a cells in the same condition as FIG. 10E were measured by qPCR (1110 in FIG. 10G and Pdx1 in FIG. 10H). FIG. 10I shows that Il10 gene activation in mouse liver was detected with the same treatments as shown in FIG. 10F (n=3). The data are means±SD. FIG. 10J shows a deep sequencing analysis used to detect indel formation in cultured N2a cells. Cells were co-transfected with either Cas9 or dCas9 together with either regular 20-bp MS2gRNAs or truncated 14-bp MS2gRNAs. Only cells co-transfected with Cas9 and regular 20-bp MSIl10 showed significant indel formation, but not cells transfected with GFP, dCas9, or Cas9 with truncated 14-bp MS2gIl10. FIG. 10K shows a deep sequencing analysis used to detect indel formation in vivo. Mouse liver samples were collected from Cas9 mice that received tail vein injections of AAV-gIl10-MPH, AAV-dgIl10-MPH (13 days post-injection), or no injection. Indels were detected in AAV-gIl10-MPH mice, but not in AAV-dgIl10-MPH mice or no-injection controls. Each treatment condition was examined in duplicate experiments.

FIGS. 11A-11G show in vivo induction of Pdx1 through CRISPR/Cas9 TGA in mouse liver generates insulin-producing cells and ameliorates hyperglycemia related to FIGS. 5A-5I. FIG. 11A shows immunofluorescence of Pdx1 and insulin in mouse liver tissues injected with AAV9-dgPdx1-T2-MPH or AAV9-dgMock-MPH (Pdx1, red; insulin, white). Scale bars, 10 μm. FIG. 11B shows that induction of Pdx1 expression in liver tissue of male Cas9 mice through CRISPR/Cas9 TGA (via tail vein injection of AAV-dgPdx1-T2-MPH) ameliorates streptozotocin (STZ)-induced hyperglycemia (n=16, AAV-dgPdx1-T2-MPH; n=12, AAV-dgMock-MPH). Glucose levels were measured from blood samples drawn from the tail. FIG. 11C shows serum insulin levels in blood samples from STZ-treated mice with either dgPdx1 or dgMock injections. FIGS. 11D-11G show in vivo activation of multiple genes using the CRISPR/Cas9 TGA system. FIG. 11D shows a schematic of the mouse Six2 gene. The arrows indicate target sites and directions of dgRNAs. Cas9 mESCs were transfected with indicated dgRNAs, and levels of Six2 expression were analyzed by qRT-PCR 4 days later (n=3). FIG. 11E shows a qRT-PCR analysis of in vivo Six2 gene induction in liver cells of Cas9 mice that received tail vein injections of AAV9-dgSix2-T5-MPH (13 days post-injection) (n=3). Gene expression fold-change was quantified relative to AAV9-dgMock-MPH controls. FIG. 11F shows immunofluorescence analyses of Six2 protein levels in liver cells of mice injected with AAV9-dgSix2-T5-MPH or AAV9-dgMock-MPH. Scale bars, 50 μm. FIG. 11G shows a qRT-PCR analysis of in vivo Pdx1 and Six2 gene induction in liver cells of Cas9 mice that received tail vein injections of AAV9-dgPdx1-T2-MPH, AAV9-dgSix2-T5-MPH, or both AAVs (13 days post-injection) (n=3). The data are means±SD.

FIGS. 12A-12F show restoration of Klotho expression by CRISPR/Cas9 TGA to treat the Mdx mouse model of Duchenne muscular dystrophy related to FIGS. 6A-6F. FIG. 12A shows in vivo TGA of Klotho in Cas9/mdx mice via facial vein injection of AAV-dgKlotho-T3-MPH at P0.5. FIG. 12B shows a qRT-PCR analysis of in vivo Klotho gene induction in muscle tissue of 4-week-old Cas9/mdx mice. Gene expression fold change was quantified relative to AAV-dgMock-MPH controls (n=3). FIG. 12C shows a representative image of TA muscles of 12-week-old mice that received dgMock or dgKlotho. FIG. 12D shows the TA muscle mass of 12-week-old mice (n=5, AAV-dgMock-MPH; n=8 AAV-dgKlotho-MPH). FIG. 12E shows the latency to fall (sec) and impulse (s, g) as latency to fall (s) x animal weight (g) in the wire hang test for 8-week-old mice (n=8, AAV9-dgMock-MPH; n=11, AAV9-dgKlotho-MPH). FIG. 12F shows the grip strength test for fore limbs and hind limbs of 8-week-old mice. (n=8, AAV9-dgMock-MPH; n=11, AAV9-dgKlotho-MPH). The data are means±SD.

FIGS. 13A-13D show physiological analyses of Mdx mice injected with AAV-dgUtrn-MPH following pathophysiology onset (3-Week-Old Mice) related to FIGS. 6A-6F. FIG. 13A shows in vivo TGA in different transgenic Cas9/mdx mice via TA and QF injection of a combination of AAV-dgUtrn-T2-MPH and AAV-dgUtrn-T16 (dgUtrn-T2+T16) at 3 weeks of age. FIG. 13B shows representative images of H&E-stained TA muscles from 4-month-old Cas9/mdx or mdx littermate mice injected with dgUtrn-T2+T16 at 3 weeks of age (two examples from each group are shown). Scale bar, 50 μm. FIG. 13C fiber size distributions of TA muscles of mdx mice injected in FIG. 13B. FIG. 13D shows immunofluorescence of Utrophin protein in mouse TA muscle injected with either dgMock or co-injected with dgUtrn-T2 and dgUtrn-T16 (Utrophin, pink; DAPI, blue). Scale bars, 50 μm.

FIGS. 14A-14F show that an AAV-SpCas9-mediated CRISPR/Cas9 TGA promotes remodeling of epigenetic marks at the Fst locus in skeletal muscle related to FIGS. 7A-7F. FIG. 14A shows in vivo TGA in wild-type mice via intramascular (IM) co-injection of AAV-SpCas9 (or AAV-SpdCas9) and AAV-dgFst-T2-MPH (dgFst) or AAV-dgMock-MPH (dgMock) into fore limb and hind limb muscle at P2.5. FIG. 14B shows a qRT-PCR analysis of in vivo fore limb and hind limb muscle samples after AAV-SpCas9 and dgFst or dgMock co-injections (21 days post-injection). Fold-change in Fst expression was quantified relative to AAV-dgMock-MPH controls (n=3). FIG. 14C shows the fold-change in Fst expression in hind limb TA muscle after IM co-injection of AAV-SpdCas9 and AAV-dgFst-T2-MPH or AAV-dgMock-MPH (n=3). The data are means±SD. FIGS. 14D-14F show co-injection of AAV-SpCas9 and AAV-dgRNA remodels epigenetic marks at the Fst target loci in vivo. FIG. 14D shows the distribution of H3K4me3, H3K27ac, and CpG islands around the Fst locus in limb tissue (UCSC genome browser). The black bars are ChIP-qPCR regions, and the red bar is the dgRNA target. FIGS. 14E-14F show a ChIP-qPCR analysis for H3K4me3 (FIG. 14E), H3K27ac (FIG. 14F), and IgG (negative control) in muscle tissue of 2.5-month-old mice that received IM injection of PBS only or co-injections of AAV-SpCas9+AAV-dgFst-T2-MPH. Relative real-time PCR values compared to input are shown (n=3). The data are means±SEM.

### SEQUENCE LISTING

The nucleic and amino acid sequences listed in the accompanying sequence listing are shown using standard letter abbreviations for nucleotide bases and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Nov. 3, 2020, 39 KB, which is incorporated by reference herein. In the accompanying sequence listing:

SEQ ID NO: 1 is an exemplary TCAG-MS2dgRNA nucleotide sequence.

SEQ ID NO: 2 is an exemplary TC5GC-MS2dgRNA nucleotide sequence.

SEQ ID NO: 3 is an exemplary TC-MS2dgRNA nucleotide sequence.

SEQ ID NO: 4 is an exemplary 5GC-MS2dgRNA nucleotide sequence.

SEQ ID NO: 5 is an exemplary MS2gRNA nucleotide sequence.

SEQ ID NO: 6 is an exemplary SE-MS2gRNA nucleotide sequence.

SEQ ID NO: 7 is an exemplary gRNA native backbone nucleotide sequence.

SEQ ID NO: 8 is an exemplary gRNA native backbone nucleotide sequence.

SEQ ID NO: 9 is an exemplary gRNA native backbone nucleotide sequence.

SEQ ID NO: 10 is an exemplary gRNA modified backbone nucleotide sequence.

SEQ ID NO: 11 is an exemplary gRNA modified backbone nucleotide sequence.

SEQ ID NO: 12 is an exemplary native MS2 binding loop nucleotide sequence.

SEQ ID NO: 13 is an exemplary modified MS2 binding loop nucleotide sequence.

SEQ ID NO: 14 is an exemplary modified MS2 binding loop nucleotide sequence.

SEQ ID NO: 15 is an exemplary modified MS2 binding loop nucleotide sequence.

SEQ ID NO: 16 is an exemplary Cas9 protein sequence.

SEQ ID NO: 17 is an exemplary dead Cas9 (dCas9) protein sequence with point mutations D10A and H840A.

SEQ ID NO: 18 is an exemplary MS2-p65-HSF1 protein sequence.

SEQ ID NOS: 19 and 20 are exemplary primer with deep sequencing adaptor nucleotide sequences.

SEQ ID NO: 21 is an exemplary 20bp-MS2gRNA nucleotide sequence.

SEQ ID NO: 22 is an exemplary 14bp-MS2gRNA (dead gRNA) nucleotide sequence.

SEQ ID NO: 23 is an exemplary 14bp-TCAG-MS2dgRNA nucleotide sequence.

SEQ ID NO: 24 is an exemplary 14bp-SE-MS2dgRNA nucleotide sequence.

SEQ ID NO: 25 is an exemplary 14bp-TC-MS2dgRNA nucleotide sequence.

SEQ ID NO: 26 is an exemplary 14bp-5GC-MS2dgRNA nucleotide sequence.

SEQ ID NO: 27 is an exemplary 14bp-TC5GC-MS2dgRNA nucleotide sequence.

SEQ ID NO: 28 is an exemplary TCAG-MS2dgRNA nucleotide sequence.

SEQ ID NO: 29 is an exemplary TC5GC-MS2dgRNA nucleotide sequence.

SEQ ID NO: 30 is an exemplary TC-MS2dgRNA nucleotide sequence.

SEQ ID NO: 31 is an exemplary 5GC-MS2dgRNA nucleotide sequence.

SEQ ID NO: 32 is an exemplary MS2gRNA nucleotide sequence.

SEQ ID NO: 33 is an exemplary SE-MS2gRNA nucleotide sequence.

SEQ ID NO: 34 is an exemplary gRNA native backbone nucleotide sequence.

SEQ ID NO: 35 is an exemplary gRNA native backbone nucleotide sequence.

SEQ ID NO: 36 is an exemplary gRNA native backbone nucleotide sequence.

SEQ ID NO: 37 is an exemplary gRNA modified backbone nucleotide sequence.

SEQ ID NO: 38 is an exemplary gRNA modified backbone nucleotide sequence.

SEQ ID NO: 39 is an exemplary native MS2 binding loop nucleotide sequence.

SEQ ID NO: 40 is an exemplary modified MS2 binding loop nucleotide sequence.

SEQ ID NO: 41 is an exemplary modified MS2 binding loop nucleotide sequence.

SEQ ID NO: 42 is an exemplary modified MS2 binding loop nucleotide sequence.

### DETAILED DESCRIPTION

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, and sequences associated with the provided GenBank® Accession numbers (as of Jun. 6, 2018), are herein incorporated by reference in their entireties.

In order to facilitate review of the various embodiments of the disclosure, the following explanations of specific terms are provided:

### I. Terms

Administration: To provide or give a subject an agent, such as a disclosed target gene activation (TGA) system or portion thereof (such as gRNA, dgRNA, Cas9 coding sequence, dCas9 coding sequence, or MS2-transcriptional activator fusion protein coding sequence, which may be part of a viral vector), by any effective route. Exemplary routes of administration include, but are not limited to, injection (such as subcutaneous, intramuscular, intradermal, intraperitoneal, intratumoral, and intravenous), transdermal, intranasal, and inhalation routes.

Adeno-associated virus (AAV): A small non-enveloped virus that can infect humans and some other primates. It can infect both nondividing and dividing cells. AAV vectors can be used as a gene therapy vector, for example, to deliver a nucleic acid molecule to a target gene using the disclosed TGA reagents and methods. Exemplary AAV vectors that can be used in the methods and compositions provided herein, include AAV1, AAV2, AAV3, AAV4, AAV5, AAV6, AAV7, AAV8, AAV9, AAV10, AAV11, AAV12, AAV-PHP.B, AAV-PHP.eB, and AAV-PHP.S. In some examples, an AAV vector containing a gRNA, dgRNA, Cas9 coding sequence, dCas9 coding sequence, or MS2-transcriptional activator fusion protein coding sequence, has tropism for a specific tissue or cell-type, for example as shown below:

| Tissue | Optimal Serotype |
|---|---|
| CNS | AAV1, AAV2, AAV4, |
| | AAV5, AAV8, AAV9, |
| | AAV-PHP.B, AAV-PHP.eB, |
| | AAV-PHP.S |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor Cells | AAV2, AAV5, AAV8 |
| RPE (Retinal | AAV1, AAV2, AAV4, |
| Pigment Epithelium) | AAV5, AAV8 |
| Skeletal Muscle | AAV1, AAV6, AAV7, |
| | AAV8, AAV9 |

Cas9: An RNA-guided DNA endonuclease enzyme that that participates in the CRISPR-Cas immune defense against prokaryotic viruses. Cas9 has two active cutting sites (HNH and RuvC), one for each strand of the double helix. An exemplary native Cas9 sequence from S. pyogenes is shown in SEQ ID NO: 16.

Catalytically inactive (deactivated or dead) Cas9 (dCas9), which has reduced or abolished endonuclease activity but still binds to dsDNA, as also encompassed by this disclosure. In some examples, a dCas9 includes one or more mutations in the RuvC and UNH nuclease domains, such as one or more of the following point mutations: D10A, E762A, D839A, H840A, N854A, N863A, and D986A (eg., based on numbering in SEQ ID NO: 16). An exemplary dCas9 sequence with D10A and H840A substitutions is shown in SEQ ID NO: 17. In one example, the dCas9 protein has mutations D10A, H840A, D839A, and N863A (see, e.g., Esvelt et al., Nat. Meth. 10:1116-21, 2013).

In some examples, Cas9 or dCas9 does not include a transcriptional activation domain, such as VP64, P65, MyoD1, HSF1, RTA, SET7/9, or any combination thereof. In other examples, Cas9 or dCas9 includes a transcriptional activation domain, such as VP64, P65, MyoD1, HSF1, RTA, SET7/9, or any combination thereof.

Cas9 sequences are publicly available. For example, GenBank® Accession Nos. nucleotides 796693 . . . 800799 of CP012045.1 and nucleotides 1100046 . . . 1104152 of CP014139.1 disclose Cas9 nucleic acids, and GenBank® Accession Nos. NP_269215.1, AMA70685.1, and AKP81606.1 disclose Cas9 proteins. In some examples, the Cas9 is a deactivated form of Cas9 (dCas9), such as one that is nuclease deficient (e.g., those shown in GenBank® Accession Nos. AKA60242.1 and KR011748.1). Activatable Cas9 proteins are provided in US Publication No. 2018-0073002-Al, incorporated herein by reference. In certain examples, Cas9 or dCas9 used in the disclosed methods or kits has at least 80% sequence identity, for example at least 85%, at least 90%, at least 95%, at least 98%, or at least 99% sequence identity to such sequences (such as SEQ ID NOS: 16 and 17 and, in some examples, wherein a variant dCas9 retains a D10A, E762A, D839A, H840A, N854A, N863A, and/or D986A substitution), and retains the ability to be used in the disclosed methods (e.g., can be used in a TGA system to increase expression of a target gene).

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, and 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

CRISPRs (clustered regularly interspaced short palindromic repeats): DNA loci containing short repetitions of base sequences. Each repetition is followed by short segments of “spacer DNA” from previous exposures to a virus. CRISPRs are found in approximately 40% of sequenced bacteria genomes and 90% of sequenced archaea. CRISPRs are often associated with cas genes that code for proteins related to CRISPRs. The CRISPR/Cas system is a prokaryotic immune system that confers resistance to foreign genetic elements, such as plasmids and phages, and provides a form of acquired immunity. CRISPR spacers recognize and cut these exogenous genetic elements in a manner analogous to RNAi in eukaryotic organisms. The modified CRISPR/Cas system disclosed herein can be used for gene regulation, specifically to activate expression, without cutting ds DNA. By delivering a dCas9 protein, dgRNA, or both, activation of expression of a target gene (or other nucleic acid molecule) can be achieved without cutting dsDNA.

Effective amount: The amount of an agent (such as the TGA reagents provided herein) that is sufficient to effect beneficial or desired results.

A therapeutically effective amount may vary depending upon one or more of: the subject and disease condition being treated, the weight and age of the subject, the severity of the disease condition, the manner of administration, and the like, which can readily be determined by one of ordinary skill in the art. The beneficial therapeutic effect can include enablement of diagnostic determinations; amelioration of a disease, symptom, disorder, or pathological condition; reducing or preventing the onset of a disease, symptom, disorder, or pathological condition; and generally counteracting a disease, symptom, disorder, or pathological condition. In one embodiment, an “effective amount” is an amount sufficient to reduce symptoms of a disease, for example, by at least 10%, at least 20%, at least 50%, at least 70%, or at least 90% (as compared to no administration of the therapeutic agent).

The term also applies to a dose that will allow for expression of an Cas13d and/or gRNA herein and that allows for targeting (e.g., detection or modification) of a target RNA.

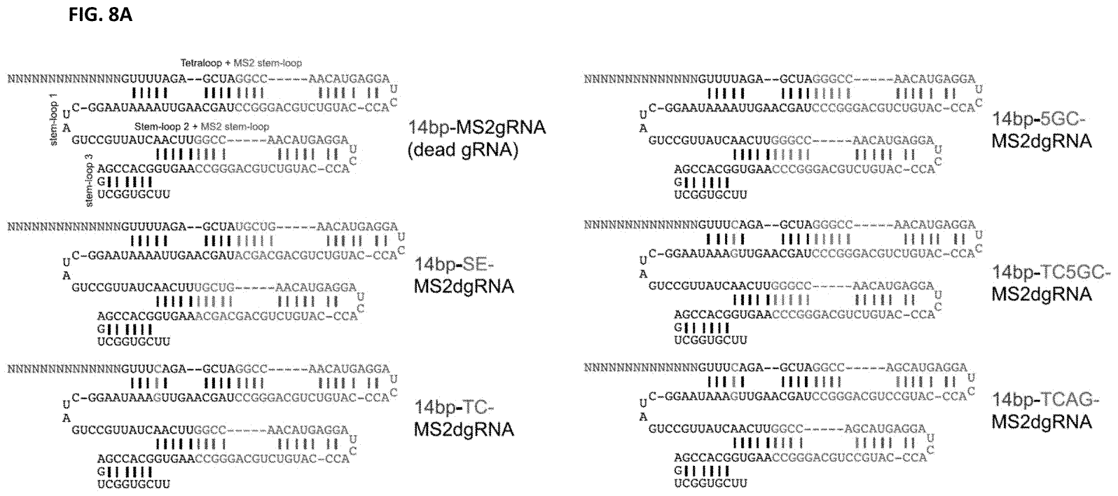

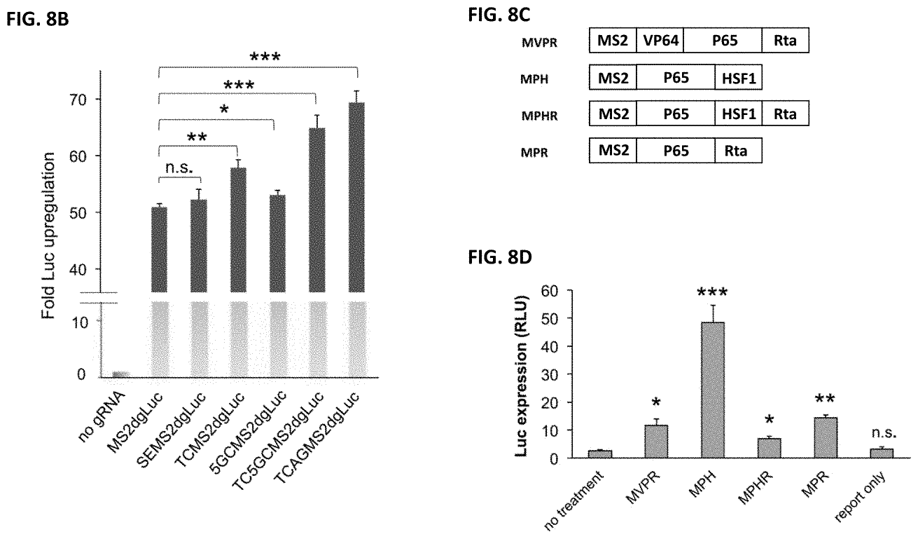

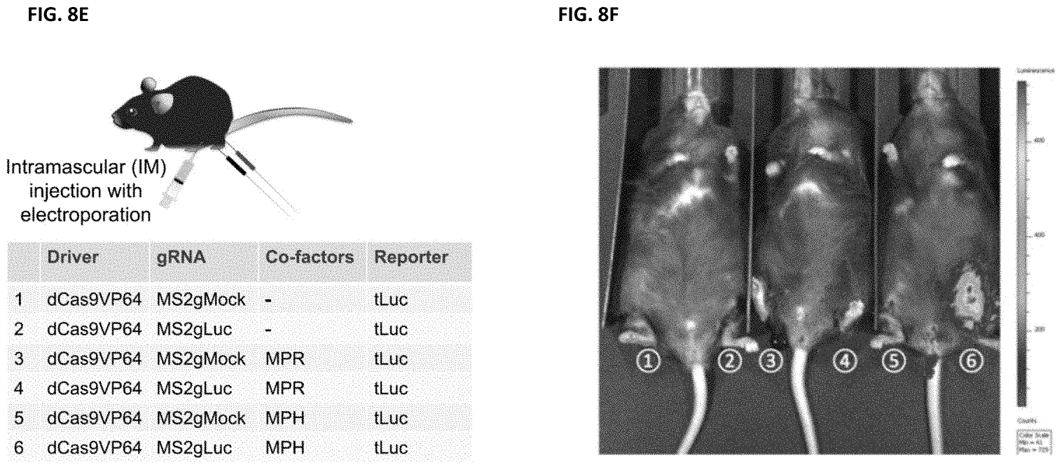

Fusion Protein: A protein that includes at least a portion of the sequence of a full-length first protein (e.g., MS2) and at least a portion of the sequence of a full-length second protein (e.g., a transcriptional activator), where the first and second proteins are different. The two different peptides can be joined directly or indirectly, for example, using a linker (such as a linker of Gly, Ser, or combinations thereof, such as GGGGS). Exemplary fusion proteins include an MS2 domain (e.g., amino acids 1-130 of SEQ ID NO: 18) fused directly or indirectly to one or more transcriptional activation domains, such as one or more of VP64, p65, MyoD1, HSF1, RTA, or SET7/9, such as an MS2-P65-HSF1 fusion protein (see SEQ ID NO: 18, and Konermann et al., Nature, 2015 Jan. 29; 517(7536):583-8). Additional examples are shown in FIG. 8C.

Guide sequence or Guide RNA (gRNA): A polynucleotide sequence used to direct a Cas9 or dCas9 protein to a target nucleic acid sequence. In some examples, the guide sequence is RNA (for example, when expressed in a cell). In some examples, the guide sequence is DNA (for example, when in a vector, such as a viral vector). The guide nucleic acid can include modified bases or chemical modifications (e.g., see Latorre et al., Angewandte Chemie 55:3548-50, 2016). In some examples, the gRNA includes two or more MS2-binding loop sequences, which can be modified from the native MS2-binding loop sequence to increase GC content and/or shorten repetitive content. In some examples, the gRNA includes two or more backbone sequences, which can be modified from the native backbone sequence to increase GC content and/or shorten repetitive content. Increasing GC content and/or shortening the repetitive content of the gRNA can be used to convert the gRNA into a dead gRNA (dgRNA), that is, a guide nucleic acid molecule that can direct a Cas9 or dCas9 protein to the target sequence, but does not induce DNA double strand break.

The term gRNA as used herein may or may not include a targeting sequence portion (i.e., portion having complementarity with a target nucleic acid sequence). Thus, it is understood that the gRNAs provided herein that do not have a targeting sequence (e.g., SEQ ID NOS: 1-6 or 28-33) can be attached to any targeting sequence of interest, such as one that has complementarity to a target nucleic acid sequence whose activated expression is desired. In some examples, the gRNA includes 14-30 nt having sufficient complementarity with a target nucleic acid sequence to hybridize with the target sequence and direct sequence-specific binding of a Cas9or dCas9 to the target nucleic acid sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 98%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some examples, the gRNA includes two or more modified MS2-binding loop sequences with increased GC content and/or decreased repetitive sequence content, two or more modified backbone sequences with increased GC content and/or shortened repetitive content, or combinations thereof. The targeting sequence can be 14-30 nt. In some examples, the gRNA includes two or more native MS2-binding loop sequences and native backbone sequences (e.g., SEQ ID NO: 1 or 28). In such cases, the targeting sequence can be 14 or 15 nt, as the shorter targeting sequence renders the gRNA dead.

In some embodiments, a gRNA molecule (without the targeting sequence of 14-30 nt, such as a targeting sequence at least about 14, 15, 16, 17, 18, 19, 20, 21, 22, 23, 24, 25, 26, 27, 28, 29, or 30 nt) is about or at least about 130, 135, 140, 141, 142, 143, 144, 145, 146, 147, 148, 149, 150, 151, 152, 153, 154, 155, or 160 nt in length. In some embodiments, a gRNA molecule (without the targeting sequence of 14-30 nt, such as a targeting sequence at least about 14, 15, 16, 17, 18, 19, 20, 21, 22, 23, 24, 25, 26, 27, 28, 29, or 30 nt) is 120-170 nucleotides (such as 135 to 160 nt, 140 to 160 nt, or 140 to 150 nt).

Increase or Decrease: A statistically significant positive or negative change, respectively, in quantity from a control value. An increase is a positive change, such as an increase at least 50%, at least 100%, at least 200%, at least 300%, at least 400%, or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples, the decrease is less than 100%, such as a decrease of no more than 90%, no more than 95%, or no more than 99%.

Isolated: An “isolated” biological component (such as a dCas9 protein or nucleic acid, gRNA, or cell containing such) has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated vectors containing a gRNA, dgRNA, nucleic acid encoding a protein (such as dCas9, Cas9, or MS2-transcriptional activator fusion protein), or cells containing such vectors, in some examples, are at least 50% pure, such as at least 75%, at least 80%, at least 90%, at least 95%, at least 98%, or at least 100% pure.

Label: A compound or composition that is conjugated directly or indirectly to another molecule (such as a nucleic acid molecule) to facilitate detection of that molecule. Specific, non-limiting examples of labels include fluorescent and fluorogenic moieties, chromogenic moieties, haptens, affinity tags, and radioactive isotopes. The label can be directly detectable (e.g., optically detectable) or indirectly detectable (for example, via interaction with one or more additional molecules that are in turn detectable).

Male-specific bacteriophage 2 (MS2): An RNA virus that includes an RNA operator hairpin that binds a coat protein (i.e., the MS2 domain or MS2 protein; e.g., amino acids 1-130 of SEQ ID NO: 18). MS2-binding hairpin aptamers (i.e., MS2 hairpins or MS2 stem loops; e.g., SEQ ID NO: 12 or SEQ ID NO: 39) and MS2 proteins have also been incorporated into synergistic activation mediator (SAM) complexes in second-generation CRISPR-Cas9 systems, and modifications of such MS2 hairpin sequences are provided herein (such as SEQ ID NOS: 13-15 and 40-42), which can be incorporated into a guide RNA, for example, to form a dead gRNA. MS2 proteins (e.g., amino acids 1-130 of SEQ ID NO: 18) have been incorporated into fusion proteins to recruit transcription factors. p Non-naturally occurring or engineered: Terms used herein interchangeably and indicate the involvement of the hand of man. The terms, when referring to nucleic acid molecules or polypeptides, indicate that the nucleic acid molecule or the polypeptide is at least substantially free from at least one other component with which they are naturally associated in nature and as found in nature. In addition, the terms can indicate that the nucleic acid molecules or polypeptides have a sequence not found in nature.

Reporter protein: Any protein whose expression is linked to expression of a gene of interest. Exemplary reporter proteins include fluorescent proteins and chemiluminescent molecules, such as infrared-fluorescent proteins (IFPs), mRFP1, mCherry, mOrange, DsRed, tdTomato, mKO, tagRFP, EGFP, mEGFP, mOrange2, maple, tagRFP-T, firefly luciferase, renilla luciferase, and click beetle luciferase (e.g., US Pat. Pub. No. 2010/0122355, incorporated herein by reference). In some examples, the reporter protein is positioned downstream of and in frame with a gene of interest, such that the reporter protein is co-expressed with the gene of interest (e.g., where a CRISPR/Cas9 target gene activation system is used, one or more reporter proteins can be positioned downstream of a target sequence such that the one or more reporter proteins, such as luciferase and/or mCherry, are co-expressed with activation of a target gene).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence (such as a coding sequence of a dCas9, Cas9, or MS2-transcriptional activator fusion protein) if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the TGA reagents provided herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Polypeptide, peptide, and protein: Refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

Promoter: An array of nucleic acid control sequences that direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

Recombinant or host cell: A cell that has been genetically altered or is capable of being genetically altered by introduction of an exogenous polynucleotide, such as a recombinant plasmid or vector. Typically, a host cell is a cell in which a vector can be propagated and its nucleic acid expressed. Such cells can be eukaryotic or prokaryotic. The term also includes any progeny of the subject host cell. It is understood that all progeny may not be identical to the parental cell because there may be mutations that occur during replication. However, such progeny are included when the term “host cell” is used.

Regulatory element: A phrase that includes promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990), which is hereby incorporated by reference in its entirety. Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

In some embodiments, a vector provided herein includes a pol III promoter (e.g., U6 and H1 promoters), a pol II promoter (e.g., the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter), or both.

Also encompassed by the term “regulatory element” are enhancer elements, such as WPRE; CMV enhancers; the R-U5′ segment in LTR of HTLV-I; SV40 enhancer; and the intron sequence between exons 2 and 3 of rabbit β-globin.

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well-known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of known protein and nucleic acid sequences and those disclosed herein are typically characterized by possession of at least about 80%, at least 90%, at least 95%, at least 96%, at least 97%, at least 98%, or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids and may possess sequence identities of at least 85% or at least 90% or at least 95%, depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, in one example, a gRNA or dgRNA nucleic acid molecule has at least 80%, at least 85%, at least 90%, at least 91%, at least 92%, at least 95%, at least 96%, at least 97%, at least 98%, or at least 99% sequence identity to SEQ ID NO: 1, 2, 3, 4, 5, or 6.

Subject: A vertebrate, such as a mammal, for example, a human. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, horse, or cow. In some examples, the subject has a disorder or genetic disease that can be treated using methods provided herein, such as a disorder that results from decreased gene expression. In some examples, the subject is a laboratory animal/organism, such as a zebrafish, Xenopus, C. elegans, Drosophila, mouse, rabbit, or rat. Tissues, cells, and their progeny of a biological entity obtained in vivo or cultured in vitro are also encompassed.

Therapeutic agent: Refers to one or more molecules or compounds that confer some beneficial effect upon administration to a subject. The beneficial therapeutic effect can include enablement of diagnostic determinations; amelioration of a disease, symptom, disorder, or pathological condition; reducing or preventing the onset of a disease, symptom, disorder, or pathological condition; and generally counteracting a disease, symptom, disorder, or pathological condition.

Transcriptional activator: A protein or protein domain that increases transcription of a nucleic acid molecule, such as a gene. Such proteins can be used in the methods and TGA systems provided herein, for example, to assist in the recruitment of co-factors and RNA polymerase for the transcription of the target gene. Such proteins and proteins domains can have a DNA binding domain and a domain for activation of transcription. These activators can be introduced into the system through attachment to Cas9, dCas9, or the gRNA. Examples of such activators include VP64, p65, myogenic differentiation 1 (MyoD1), heat shock transcription factor (HSF) 1, RTA, SET7/9, or any combination thereof (such as p65 and HSF1).

Transduced, Transformed, and Transfected: A virus or vector “transduces” a cell when it transfers nucleic acid molecules into a cell. A cell is “transformed” or “transfected” by a nucleic acid transduced into the cell when the nucleic acid becomes stably replicated by the cell, either by incorporation of the nucleic acid into the cellular genome or by episomal replication.

These terms encompasses all techniques by which a nucleic acid molecule can be introduced into such a cell, including transfection with viral vectors, transformation with plasmid vectors, and introduction of naked DNA by electroporation, lipofection, particle gun acceleration, and other methods in the art. In some examples, the method is a chemical method (e.g., calcium-phosphate transfection), physical method (e.g., electroporation, microinjection, or particle bombardment), fusion (e.g., liposomes), receptor-mediated endocytosis (e.g., DNA-protein complexes or viral envelope/capsid-DNA complexes), and biological infection by viruses, such as recombinant viruses (Wolff, J. A., ed, Gene Therapeutics, Birkhauser, Boston, USA, 1994). Methods for the introduction of nucleic acid molecules into cells are known (e.g., see U.S. Pat. No. 6,110,743). These methods can be used to transduce a cell with the disclosed agents to activate expression.

Transgene: An exogenous gene.

Treating, Treatment, and Therapy: Any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters, including the results of a physical examination, blood and other clinical tests, and the like. For prophylactic benefit, the disclosed compositions may be administered to a subject at risk of developing a particular disease, condition, or symptom or to a subject reporting one or more of the physiological symptoms of a disease, even though the disease, condition, or symptom may not have yet been manifested.

Under conditions sufficient for: A phrase that is used to describe any environment that permits a desired activity. In one example, the desired activity is expression of a gRNA or dgRNA disclosed herein in combination with other necessary elements (e.g., Cas9, dCas9, or MS2-transcriptional activator fusion protein), for example, to enhance expression of a target nucleic acid.

Upregulated: When used in reference to the expression of a molecule, such as a target nucleic acid molecule (e.g., gene), refers to any process that results in an increase in production of the target nucleic acid molecule. In some examples, the target nucleic acid molecule is a gene. In some examples, the target nucleic acid molecule is DNA. In some examples, the target nucleic acid molecule is RNA, such as mRNA, miRNA, rRNA, tRNA, nuclear RNA, non-coding RNA, and structural RNA. In some examples, upregulation or activation of a target nucleic acid molecule includes processes that increase translation of the target RNA and thus can increase the presence of corresponding proteins. The disclosed TGA system can be used to upregulate any target nucleic acid molecule of interest.

Upregulation includes any detectable increase in the target nucleic acid molecule or corresponding product thereof, such as RNA or protein. In certain examples, detectable target nucleic acid expression in a cell or cell free system (such as a cell expressing a gRNA or dgRNA provided herein with Cas9 or dCas9) increases by at least 20%, at least 30%, at least 40%, at least 50%, at least 60%, at least 70%, at least 75%, at least 80%, at least 90%, at least 95%, at least 100%, at least 200%, at least 400%, or at least 500% as compared to a control (such an amount of target nucleic acid molecule detected in a corresponding untreated normal cell or sample). In one example, a control is a relative amount of expression in a normal cell (e.g., a non-recombinant cell that does not include gRNA or dgRNA provided herein with Cas9 or dCas).

Vector: A nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that comprise one or more free ends or no free ends (e.g., circular); nucleic acid molecules that comprise DNA, RNA, or both; and other varieties of polynucleotides (e.g., LNAs).

A vector can include nucleic acid sequences that permit it to replicate in a host cell, such as an origin of replication. A vector can also include one or more selectable marker genes and other genetic elements. An integrating vector is capable of integrating itself into a host nucleic acid. An expression vector is a vector that contains the necessary regulatory sequences to allow transcription and translation of inserted gene or genes.

One type of vector is a “plasmid,” which refers to a circular double-stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and, thereby, are replicated along with the host genome.

Certain vectors are capable of directing the expression of genes to which they are operatively-linked. Such vectors are referred to herein as “expression vectors.” Common expression vectors are often in the form of plasmids. Recombinant expression vectors can comprise a nucleic acid provided herein (such as a gRNA, dgRNA, or nucleic acid encoding an protein, such as Cas9, dCas9, or MS2-transcriptional activator fusion protein) in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). It will be appreciated by those skilled in the art that the design of the expression vector can depend on such factors as the choice of the host cell to be transformed, the level of expression desired, etc. A vector can be introduced into host cells to, thereby, produce transcripts, proteins, or peptides, including fusion proteins or peptides, encoded by nucleic acids as described herein.

### II. Overview of Several Embodiments

Regulating the epigenome aids in treating human diseases that have not been cured using traditional drug strategies (Heerboth et al., 2014; Hunter, 2015). Described herein is CRISPR/Cas9 TGA that can transcriptionally activate target genes in vivo by modulating histone marks rather than editing DNA sequences. Without being bound by theory, the in vivo CRISPR/Cas9 TGA herein indirectly induces epigenetic remodeling by recruiting the transcriptional machinery, not by directly recruiting epigenetic modulators. This in vivo CRISPR/Cas9 TGA altered target gene expression in vivo to generate physiologically relevant phenotypes without causing DSBs.

AAVs aid in in vivo gene delivery. A split Cas9 AAV system, which relies on the trans-splicing machinery, was previously described to circumvent the capacity limitation of AAV vectors (Chew et al., 2016). However, the modest levels of in vivo TGA achievable with the split system are not sufficient to induce phenotypic change. The in vivo CRISPR/Cas9 TGA described herein, which utilizes a modified CRISPR/Cas9 machinery and a co-transcriptional complex, can 1) rescue levels of gene expression (e.g., restore Klotho levels following acute kidney injury or in the mdx model), 2) compensate for genetic defects (e.g., overexpress Utrophin to compensate for loss of Dystrophin), and 3) alter cell fate by inducing transdifferentiation factors (e.g., generate insulin-producing cells by ectopically expressing Pdx1).

Previous research has shown partially restored dystrophin gene function in models of DMD using CRISPR/Cas9 technology by directly removing mutated exons to create a shortened dystrophin gene (Long et al., 2016; Nelson et al., 2016; Tabebordbar et al., 2016). However, this approach is not likely effective where specific exons may be essential for protein function and, therefore, cannot be removed to ameliorate the disease. In addition, this approach generates DSBs, which can create unwanted genetic mutations, a significant problem for the gene therapy field (Schaefer et al., 2017). In contrast, the in vivo CRISPR/Cas9 TGA described herein does not generate DNA breaks. Furthermore, AAV-mediated delivery of CRISPR-Cas9 does not induce extensive cellular damage in vivo (Chew et al., 2016).

The in vivo TGA system described herein can be used to transcriptionally activate endogenous genes (either single genes or combinations of genes), including large genes. This system can be used to express genes to compensate for disease-associated genetic mutations or to overexpress long non-coding RNAs or GC-rich genes to reveal their biological functions, which has been a problem in the field until now (La Russa and Qi, 2015; Vora et al., 2016). Finally, combined loss- and gain-of-function manipulations can be applied to rapidly establish epistatic relationships between genes in vivo. Thus, in vivo CRISPR/Cas9-mediated gene activation systems described herein are versatile and efficient tools for in vivo biomedical research and as a targeted epigenetic approach for treating a wide range of human diseases.

### III. Guide Nucleic Acid Molecules

Provided herein are guide nucleic acid molecules. The term guide RNA (gRNA) is used throughout the application, but one skilled in the art will recognize that the guide RNA is actually DNA when present in a vector (e.g., AAV vector) (that is “T” will be used instead of “U”), which is transcribed as RNA when expressed in a cell. Thus, although particular SEQ ID NOS herein show “T” for gRNAs or parts thereof, one skilled in the art will recognize that, when expressed, the “T” will become a “U”.

In addition, in some examples, a nucleic acid molecule is described as a gRNA, but does not include the region having complementarity to the target sequence. It is understood that such gRNA molecules can be attached at their 5′-end to any targeting sequence of interest (such as one of 14-30 bp, having sufficient complementarity to hybridize to a target sequence).

In one example, a gRNA includes the structure A-B-C-D-E, wherein A is the 5′-end and E is the 3′-end of the molecule. For example, the gRNA can include a first region (e.g., A, in A-B-C-D-E) that includes a tetraloop backbone sequence having at least 90%, at least 95%, at least 98%, at least 99%, or 100% sequence identity to the sequence gttttagagcta (SEQ ID NO: 7) or guuuuagagcua (SEQ ID NO: 34). The second region (e.g., B, in A-B-C-D-E) is linked to the first and to a third region (e.g., is in between the region A and region C), and includes a modified MS2-binding loop sequence. The third region (e.g., C, in A-B-C-D-E) is linked to the second region and to a fourth region (e.g., is in between the region B and region D), and includes a stem-loop 1 and stem-loop 2 backbone sequence having at least 90%, at least 95%, at least 98%, at least 99%, or 100% sequence identity to the sequence tagcaagttaaaataaggctagtccgttatcaactt (SEQ ID NO: 8) or uagcaaguuaaaauaaggcuaguccguuaucaacuu (SEQ ID NO: 35). The fourth region (e.g., D, in A-B-C-D-E) linked to the third region and to the fifth region (e.g., is in between the region C and region E), and includes the modified MS2-binding loop sequence (e.g., is identical to the second region). The fifth region (e.g., E, in A-B-C-D-E) is at the 3′-end of the gRNA, is linked to the fourth region, and includes a stem-loop 3 backbone sequence including at least 90%, at least 95%, at least 98%, at least 99%, or 100% sequence identity to aagtggcaccgagtcggtgctt (SEQ ID NO: 9) or aaguggcaccgagucggugcuu (SEQ ID NO: 36). The modified MS2-binding loop sequences of the gRNA include at least two nucleotide changes to the native MS2-binding loop sequence ggccaacatgaggatcacccatgtctgcagggcc (SEQ ID NO: 12) or ggccaacaugaggaucacccaugucugcagggcc (SEQ ID NO: 39), thereby increasing the GC content and/or shortening the repetitive content of the modified MS2-binding loop sequence relative to the native MS2-binding loop sequence. For example, the modified MS2-binding loop sequences can include 2, 3, 4, 5, 6, 7, 8, 9, or 10 nucleotide changes to the native MS2-binding loop sequence ggccaacatgaggatcacccatgtctgcagggcc (SEQ ID NO: 12) or ggccaacaugaggaucacccaugucugcagggcc (SEQ ID NO: 39) that increase the GC content of the native sequence, such as an increase of about 5%, 10%, 20%, 30%, 40%, or 50% (such as about 5 to 20% or 10 to 30%) and/or shorten repetitive content, such as a decrease of about 5%, 10%, 20%, 30%, 40%, or 50% (such as about 5 to 10% or 10 to 30%). In some examples, the GC content of a nucleic acid molecule is increased by adding “G” and/or “C” nucleotides to the molecule, substituting a native “A” to a “G”, substituting a native “T” or “U” to a “C”, or combinations thereof. In some examples, the repetitive content is shortened or decreased by deleting one or more repetitive nucleotides (e.g., the string of 4 Ts at nucleotides 2-5 of SEQ ID NO: 5 is shortened to a string of 3 Ts at nucleotides 2-4 of SEQ ID NO: 1). In some examples, the modified MS2-binding loop sequence comprises or consists of the sequence tgctgaacatgaggatcacccatgtctgcagcagca (SEQ ID NO: 13), gggccaacatgaggatcacccatgtctgcagggccc (SEQ ID NO: 14), ggccagcatgaggatcacccatgcctgcagggcc (SEQ ID NO: 15), ugcugaacaugaggaucacccaugucugcagcagca (SEQ ID NO: 40), gggccaacaugaggaucacccaugucugcagggccc (SEQ ID NO: 41), or ggccagcaugaggaucacccaugccugcagggcc (SEQ ID NO: 42). In some examples, the first region includes a U to C substitution, and the third region includes a A to G substitution. In some examples, the first region comprises or consists of the sequence gtttcagagcta (SEQ ID NO: 10) or guuucagagcua (SEQ ID NO: 37), and the third region comprises or consists of the sequence tagcaagttgaaataaggctagtccgttatcaactt (SEQ ID NO: 11) or uagcaaguugaaauaaggcuaguccguuaucaacuu (SEQ ID NO: 38). In some examples, the gRNA comprises or consists of the sequence of SEQ ID NO: 1, SEQ ID NO: 2, SEQ ID NO: 4, SEQ ID NO: 28, SEQ ID NO: 29, or SEQ ID NO: 31.

In another example, a gRNA can include a first region at the 5′-end (e.g., A, in A-B-C-D-E), which includes a first modified backbone sequence having at least one nucleotide change to the native tetraloop backbone sequence gttttagagcta (SEQ ID NO: 7) or guuuuagagcua (SEQ ID NO: 34). The second region (e.g., B, in A-B-C-D-E) is linked to the first region and to a third region (e.g., is in between region A and region C) and includes an MS2-binding loop sequence (such as ggccaacatgaggatcacccatgtctgcagggcc; SEQ ID NO: 12) or ggccaacaugaggaucacccaugucugcagggcc (SEQ ID NO: 39). The third region (e.g., C, in A-B-C-D-E) is linked to the second and to a fourth region (e.g., is in between region B and region D) and includes a second modified backbone sequence having at least one nucleotide change to the native stem-loop 1 and stem-loop 2 backbone sequence tagcaagttaaaataaggctagtccgttatcaactt (SEQ ID NO: 8) or uagcaaguuaaaauaaggcuaguccguuaucaacuu (SEQ ID NO: 35). The fourth region (e.g., D, in A-B-C-D-E) is linked to the third and to the fifth regions (e.g., is in between region C and region E) and includes the MS2-binding loop sequence (such as ggccaacatgaggatcacccatgtctgcagggcc; SEQ ID NO: 12) or ggccaacaugaggaucacccaugucugcagggcc (SEQ ID NO: 39). The fifth region (e.g., E, in A-B-C-D-E) is at the 3′-end of the gRNA, is linked to the fourth region, and includes a stem-loop 3 backbone sequence having at least 90%, at least 95%, at least 98%, at least 99%, or 100% sequence identity to aagtggcaccgagtcggtgctt (SEQ ID NO: 9) or aaguggcaccgagucggugcuu (SEQ ID NO: 36). The at least one ribonucleotide change in the first and second backbone sequence increases the GC content of the first and second modified backbone sequences relative to the native backbone sequences. For example, a first modified backbone sequence can include 1, 2, 3, 4, or 5 nucleotide changes to the native backbone sequence gttttagagcta (SEQ ID NO: 7) or guuuuagagcua (SEQ ID NO: 34) that increases the GC content of the native sequence, such as an increase of about 5%, 10%, 20%, 30%, 40%, or 50% (such as about 5 to 20% or 10 to 30%) and/or shorten repetitive content, such as a decrease of about 5%, 10%, 20%, 30%, 40%, or 50% (such as about 5 to 10% or 10 to 30%). For example, the second modified backbone sequence can include 1, 2, 3, 4, 5, 6, 7, 8, 9, or 10 nucleotide changes to native backbone sequence tagcaagttaaaataaggctagtccgttatcaactt (SEQ ID NO: 8) or uagcaaguuaaaauaaggcuaguccguuaucaacuu (SEQ ID NO: 35) that increases the GC content of the native sequence, such as an increase of about 5%, 10%, 20%, 30%, 40%, or 50% (such as about 5 to 20% or 10 to 30%). In some examples, the GC content of a nucleic acid molecule is increased by adding “G” and/or “C” nucleotides to the molecule, substituting a native “A” to a “G”, substituting a native “T” or “U” to a “C”, or combinations thereof. In some examples, the first modified backbone sequence includes a U to C substitution, and the second modified backbone sequence includes an A to G substitution. In some examples, the first region comprises or consists of the sequence gtttcagagcta (SEQ ID NO: 10) or guuucagagcua (SEQ ID NO: 37), and the third region comprises or consists of the sequence tagcaagttgaaataaggctagtccgttatcaactt (SEQ ID NO: 11) or uagcaaguugaaauaaggcuaguccguuaucaacuu (SEQ ID NO: 38). In some examples, the gRNA includes the gRNA of any of claims 2 to 7, wherein the gRNA comprises or consists of the sequence of SEQ ID NO: 3 or SEQ ID NO: 30.

As discussed above, the disclosed gRNA molecules can be attached at their 5′-end to any targeting sequence of interest (such as one of 14-20 bp having sufficient complementarity to hybridize to a target sequence). Thus, the targeting sequence is a variable portion of the guide sequence. Thus, in one example, the gRNA includes the structure T-A-B-C-D-E, wherein T (targeting sequence) is the 5′-end and E is the 3′-end. Thus, the gRNAs provided herein can include a sixth region at the 5′-end of the gRNA, which is linked at its 3′-end to the 5′ end of the first region of the gRNA (e.g., A in T-A-B-C-D-E). The sixth region (e.g., T in T-A-B-C-D-E) includes sufficient complementarity to a target nucleic acid molecule to hybridize to the target and is about 14 to 20 nucleotides (or ribonucleotides) in length, such as 14, 15, 16, 17, 18, 19, or 20 nucleotides (or ribonucleotides) in length. In some examples, the gRNA is a dead gRNA, wherein the sixth region (e.g., T in T-A-B-C-D-E) is about 14 or 15 nucleotides (or ribonucleotides) in length. In some examples, a targeting sequence has 100% complementarity to a target nucleic acid (or region of the DNA or RNA to be targeted), but a targeting sequence can have less than 100% complementarity to a target nucleic acid molecule, such as at least 80%, at least 85%, at least 90%, at least 95%, at least 98%, or at least 99% complementarity to a target nucleic acid molecule. The targeting sequence in some examples is complementary to a sequence near the transcriptional start site of the endogenous target nucleic acid molecule, for example, in the promoter region of the target nucleic acid molecule. In one example, the targeting sequence is complementary to a sequence at least within 10 nt, 25 nt, 50 nt, 60 nt, 70 nt, 80 nt, 90 nt, 100 nt, 110 nt, 120 nt, 130 nt, 140 nt, 150 nt, 175 nt, 200 nt, 300 nt, 400 nt, or 500 nt of the transcriptional start site. In some examples, the target nucleic acid molecule is a gene whose decreased expression results in a disease or disorder in a mammal.

Exemplary guide RNA molecules are shown below, all having the structure A-B-C-D-E (alternating underlines and bold to make the regions clear). MS2gRNA (SEQ ID NO: 5 or 32) can be converted into a dead gRNA (dgRNA) by attaching, at the 5′-end of the gRNA, a sequence of 14 or 15 nt that is complementary to the target nucleic acid. The other gRNAs shown below (SEQ ID NOS: 1-4, 6, 28-31, and 33) are dgRNAs by virtue of their GC substitutions and/or shortened repetitive content in the backbone and/or MS2 binding loop sequence. Thus, any of SEQ ID NOS: 1-4, 6, 28-31, or 33 can further include at their 5′-end a sequence of 14 to 30 nt that is complementary to the target nucleic acid.

| | Sequence |
|---|---|
| MS2gRNA | gttttagagctaggccaacatgaggatcacccatgt |
| | ctgcagggcctagcaagttaaaataaggctagtccg |
| | ttatcaacttggccaacatgaggatcacccatgtct |
| | gcagggccaagtggcaccgagtcggtgcttttt |
| | (SEQ ID NO: 5) |
| | or |
| | guuuuagagcuaggccaacaugaggaucacccaugu |
| | cugcagggccuagcaaguuaaaauaaggcuaguccg |
| | uuaucaacuuggccaacaugaggaucacccaugucu |
| | gcagggccaaguggcaccgagucggugcuuuuu |
| | (SEQ ID NO: 32) |
| SE- | gttttagagctatgctgaacatgaggatcacccatg |
| MS2gRNA | tctgcagcagcatagcaagttaaaataaggctagtc |
| | cgttatcaactttgctgaacatgaggatcacccatg |
| | tctgcagcagcaaagtggcaccgagtcggtgctttt |
| | tt (SEQ ID NO: 6) |
| | or |
| | guuuuagagcuaugcugaacaugaggaucacccaug |
| | ucugcagcagcauagcaaguuaaaauaaggcuaguc |
| | cguuaucaacuuugcugaacaugaggaucacccaug |
| | ucugcagcagcaaaguggcaccgagucggugcuuuu |
| | uu (SEQ ID NO: 33) |
| TC- | gtttcagagctaggccaacatgaggatcacccatgt |
| MS2gRNA | ctgcagggcctagcaagttgaaataaggctagtccg |
| | ttatcaacttggccaacatgaggatcacccatgtct |
| | gcagggccaagtggcaccgagtcggtgcttttt |
| | (SEQ ID NO: 3) |
| | or |
| | guuucagagcuaggccaacaugaggaucacccaugu |
| | cugcagggccuagcaaguugaaauaaggcuaguccg |
| | uuaucaacuuggccaacaugaggaucacccaugucu |
| | gcagggccaaguggcaccgagucggugcuuuuu |
| | (SEQ ID NO: 30) |
| 5GC- | gttttagagctagggccaacatgaggatcacccatg |
| MS2gRNA | tctgcagggccctagcaagttaaaataaggctagtc |
| | cgttatcaacttgggccaacatgaggatcacccatg |
| | tctgcagggcccaagtggcaccgagtcggtgctttt |
| | t (SEQ ID NO: 4) |
| | or |
| | guuuuagagcuagggccaacaugaggaucacccaug |
| | ucugcagggcccuagcaaguuaaaauaaggcuaguc |
| | cguuaucaacuugggccaacaugaggaucacccaug |
| | ucugcagggcccaaguggcaccgagucggugcuuuu |
| | u (SEQ ID NO: 31) |
| TC5GC- | gtttcagagctagggccaacatgaggatcacccatg |
| MS2gRNA | tctgcagggccctagcaagttgaaataaggctagtc |
| | cgttatcaacttgggccaacatgaggatcacccatg |
| | tctgcagggcccaagtggcaccgagtcggtgctttt |
| | t (SEQ ID NO: 2) |
| | or |
| | guuucagagcuagggccaacaugaggaucacccaug |
| | ucugcagggcccuagcaaguugaaauaaggcuaguc |
| | cguuaucaacuugggccaacaugaggaucacccaug |
| | ucugcagggcccaaguggcaccgagucggugcuuuu |
| | u (SEQ ID NO: 29) |
| TCAG- | gtttcagagctaggccagcatgaggatcacccatgc |
| MS2gRNA | ctgcagggcctagcaagttgaaataaggctagtccg |
| | ttatcaacttggccagcatgaggatcacccatgcct |
| | gcagggccaagtggcaccgagtcggtgcttttt |
| | (SEQ ID NO: 1) |
| | or |
| | guuucagagcuaggccagcaugaggaucacccaugc |
| | cugcagggccuagcaaguugaaauaaggcuaguccg |
| | uuaucaacuuggccagcaugaggaucacccaugccu |
| | gcagggccaaguggcaccgagucggugcuuuuu |
| | (SEQ ID NO: 28) |

Thus, also provided are isolated nucleic acid molecules having at least 90%, at least 95%, at least 98%, at least 99%, or 100% sequence identity to SEQ ID NO: 1-4, 6, 28-31, or 33. In some examples, an isolated nucleic acid molecule having at least 90%, at least 95%, at least 98%, at least 99%, or 100% sequence identity to SEQ ID NO: 5 or 32 further includes at the 5′-end, a sequence of 14 or 15 nt that is complementary to a target nucleic acid. In some examples, an isolated nucleic acid molecule having at least 90%, at least 95%, at least 98%, at least 99%, or 100% sequence identity to SEQ ID NOS: 1-4, 6, 28-31, or 33 can further include at its 5′-end a sequence of 14 to 30 nt that is complementary to the target nucleic acid. In some examples, such isolated nucleic acid molecules are part of a vector, such as a viral vector, such as an AAV vector.

The ability of a guide sequence to direct sequence-specific binding of a CRISPR complex to a target nucleic acid molecule may be assessed by any suitable assay. For example, the components of a CRISPR system sufficient to form a CRISPR complex, including the guide sequence to be tested, may be provided to a host cell having the corresponding target nucleic acid molecule, such as by transfection with vectors encoding the components of the CRISPR sequence, followed by an assessment of enhanced expression of the target sequence. Other assays are possible, and will occur to those skilled in the art.

The disclosed guide nucleic acid molecules can be used in the methods, compositions, and kits provided herein. Such guide nucleic acid molecules can include naturally occurring or non-naturally occurring nucleotides or ribonucleotides (such as LNAs or other chemically modified nucleotides or ribonucleotides, for example, to protect a guide RNA from degradation). In some examples, the guide sequence is RNA. In some examples, the guide sequence is DNA, for example, when part of a vector, such as a viral vector. The guide nucleic acid can include modified bases or chemical modifications (e.g., see Latorre et al., Angewandte Chemie 55:3548-50, 2016). A guide sequence directs a Cas9 or dCas9 protein to a target nucleic acid, thereby enhancing expression of the targeted nucleic acid.

A. Vectors that Include Guide Nucleic Acid Molecules

Also provided are vectors, such as a viral vector or plasmid (e.g., retrovirus, lentivirus, adenovirus, adeno-associated virus, or herpes simplex virus), which include a guide nucleic acid molecule provided herein. Exemplary vectors are described herein. In one example the vector is an AAV vector, such as an AAV9 vector. In some examples, the AAV vector has tropism for a specific tissue or cell-type. In some examples, the guide nucleic acid molecule is operably linked to a promoter or expression control element (examples of which are provided elsewhere in this application). The vectors can include other elements, such as a gene encoding a selectable marker, such as an antibiotic, such as puromycin, hygromycin, or a detectable marker such as GFP, other fluorophore, or a luciferase protein. Such vectors can include naturally occurring or non-naturally occurring nucleotides or ribonucleotides. Such vectors can be used in the methods, compositions, and kits provided herein.

B. Cells that Include Guide Nucleic Acid Molecules

Cells that include one or more guide nucleic acid molecules provided herein are provided. Such recombinant cells can be used in the methods, compositions, and kits provided herein. In some examples, such cells also include a Cas9 or dCas9 protein. In some examples, such cells also include an MS-transcriptional activator fusion protein. Guide nucleic acid molecules as well as nucleic acid molecules encoding a Cas9, a dCas9, and/or an MS-transcriptional activator fusion protein can be introduced into cells to generate transformed (e.g., recombinant) cells. In some examples, such cells are generated by introducing Cas9, dCas9, and/or MS-transcriptional activator fusion protein and one or more guide molecules (e.g., gRNAs or dgRNAs) into the cell, for example, as a ribonucleoprotein (RNP) complex.

Such recombinant cells can be eukaryotic or prokaryotic. Examples of such cells include, but are not limited to, bacteria, archaea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, Drosophila cells, C. elegans cells, Xenopus cells, SF9 cells, C129 cells, 293 cells, Neurospora, and immortalized mammalian cell lines (e.g., Hela cells, myeloid cell lines, and lymphoid cell lines).

In one example, the cell is a prokaryotic cell, such as a bacterial cell, such as E. coli.

In one example, the cell is a eukaryotic cell, such as a mammalian cell, such as a human cell. In one example, the cell is primary eukaryotic cell, a stem cell, a tumor/cancer cell, a circulating tumor cell (CTC), a blood cell (e.g., T cell, B cell, NK cell, Tregs, etc.), hematopoietic stem cell, specialized immune cell (e.g., tumor-infiltrating lymphocyte or tumor-suppressed lymphocytes), a stromal cell in the tumor microenvironment (e.g., cancer-associated fibroblasts, etc.), pancreatic cell, kidney cell, or muscle cell. In one example, the cell is a brain cell (e.g., neurons, astrocytes, microglia, retinal ganglion cells, rods/cones, etc.) of the central or peripheral nervous system).

In one example, a cell is part of (or obtained from) a biological sample, such as a biological specimen containing genomic DNA, RNA (e.g., mRNA), protein, or combinations thereof obtained from a subject. Examples include, but are not limited to, peripheral blood, serum, plasma, urine, saliva, sputum, tissue biopsy, fine needle aspirate, surgical specimen, and autopsy material.

In one example, the cell is from a tumor, such as a hematological tumor (e.g., leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (including low-, intermediate-, and high-grade), multiple myeloma, Waldenström's macroglobulinemia, heavy chain disease, myelodysplastic syndrome, mantle cell lymphoma, and myelodysplasia) or solid tumor (e.g., sarcomas and carcinomas: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, and bladder carcinoma as well as CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma)).

C. Compositions & Kits

Also provided are compositions and kits that include one or more guide nucleic acid molecules (e.g., gRNA or dgRNA) provided herein. In one example, the compositions include one or more guide nucleic acid molecules (e.g., gRNA or dgRNA) provided herein (such as SEQ ID NO: 1-4, 6, 28-31, or 33 and, optionally, a targeting sequence) and a pharmaceutically acceptable carrier (e.g., saline, water, or PBS). The one or more guide nucleic acid molecules can be present in a vector, such as a viral vector that is part of the composition. In some examples, the one or more guide nucleic acid molecules are present in a cell that is part of the composition. In some examples, the composition is a liquid, a lyophilized powder, or cryopreserved.

The compositions are, optionally, suitable for formulation and administration in vitro or in vivo. Suitable carriers and their formulations are described in Remington: The Science and Practice of Pharmacy, 22^{nd} Edition, Loyd V. Allen et al., editors, Pharmaceutical Press (2012). Pharmaceutically acceptable carriers include materials that are not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

In some embodiments, the disclosed compositions for administration are dissolved in a pharmaceutically acceptable carrier, such as an aqueous carrier. A variety of aqueous carriers can be used, e.g., buffered saline and the like. These solutions can be sterile and generally free of undesirable matter. These compositions may be sterilized. The compositions may contain pharmaceutically acceptable auxiliary substances as required to approximate physiological conditions, such as pH adjusting and buffering agents, toxicity adjusting agents, and the like, for example, sodium acetate, sodium chloride, potassium chloride, calcium chloride, sodium lactate, and the like. The concentration of active agent in these formulations can vary and can be selected primarily based on fluid volumes, viscosities, body weight, and the like in accordance with the particular mode of administration selected and the subject's needs.

Pharmaceutical formulations can be prepared by mixing the disclosed nucleic acid molecules (e.g., vectors), proteins, or combinations thereof having the desired degree of purity with optional pharmaceutically acceptable carriers, excipients, or stabilizers. Such formulations can be lyophilized formulations or aqueous solutions.

Acceptable carriers, excipients, or stabilizers are nontoxic to recipients at the dosages and concentrations used. Acceptable carriers, excipients, or stabilizers can be acetate, phosphate, citrate, and other organic acids; antioxidants (e.g., ascorbic acid) preservatives, and low molecular weight polypeptides; proteins, such as serum albumin or gelatin, or hydrophilic polymers, such as polyvinylpyllolidone; and amino acids, monosaccharides, disaccharides, and other carbohydrate,s including glucose, mannose, or dextrins; chelating agents; ionic and non-ionic surfactants (e.g., polysorbate); salt-forming counter-ions, such as sodium; metal complexes (e.g. Zn-protein complexes); and/or non-ionic surfactants.

Formulations suitable for oral administration can include (a) liquid solutions, such as an effective amount of the disclosed nucleic acid molecules (e.g., vectors), proteins, or combinations thereof, suspended in diluents, such as water, saline, or PEG 400; (b) capsules, sachets or tablets, each containing a predetermined amount of the active ingredient, as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers.

The disclosed nucleic acid molecules (e.g., vectors), proteins, or combinations thereof, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the provided methods, compositions can be administered, for example, by intravenous infusion, orally, topically, intraperitoneally, intravesically, intratumorally, or intrathecally. Parenteral administration, intratumoral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced or infected with the disclosed nucleic acids for ex vivo therapy can also be administered intravenously or parenterally as described above.

The pharmaceutical preparation can be in unit dosage form. In such form, the preparation is subdivided into unit doses containing appropriate quantities of the active component. Thus, the pharmaceutical compositions can be administered in a variety of unit dosage forms depending upon the method of administration. For example, unit dosage forms suitable for oral administration include, but are not limited to, powder, tablets, pills, capsules, and lozenges.

In some embodiments, the compositions include at least two different gRNAs or dgRNAs, such as those that target different genes for activation.

Also provided are kits that include one or more gRNAs provided herein (which may be part of a vector, such as an AAV vector, and/or may be present in a cell, such as a mammalian cell). The kits can further include a nucleic acid encoding a Cas9 protein or dCas9 protein (which may be part of a vector, such as an AAV vector, and/or may be present in a cell, such as a mammalian cell). In some examples, the kits further include a Cas9 protein or dCas9 protein. The kits can further include a nucleic acid encoding an MS2-transcriptional activator fusion protein (such as MS2-p65-HSF1), which may be part of a vector (such as an AAV vector) and/or may be present in a cell, such as a mammalian cell. In some examples, the nucleic acid encoding a Cas9 protein or dCas9 protein and the nucleic acid encoding an MS2-transcriptional activator fusion protein are part of a single viral vector (e.g., AAV). In some examples, the nucleic acid encoding an MS2-transcriptional activator fusion protein encodes MS2-p65-HSF1, such as a sequence encoding a protein sequence having at least 90%, at least 95%, at least 98%, at least 99%, or 100% sequence identity to SEQ ID NO: 18.

In one example, the composition or kit includes an RNP complex (e.g., a TGA complex) composed of one or more Cas9 or dCas9 proteins and one or more disclosed dgRNA or gRNA molecules, and one or more transcriptional activators. In one example, the composition or kit includes a vector encoding a Cas9 or dCas9 protein and a vector encoding one or more disclosed dgRNA or gRNA molecules and encoding an MS2-transcriptional activator fusion protein. In one example, the composition or kit includes a cell, such as a bacterial cell or eukaryotic cell, that includes aCas9 or dCas9 protein, a Cas9 or dCas9 protein coding sequence, a dgRNA or gRNA molecule, a nucleic acid encoding an MS2-transcriptional activator fusion protein, MS2-transcriptional activator fusion protein (such as SEQ ID NO: 18), or combinations thereof. In one example, the composition or kit includes a cell-free system that includes Cas9 or dCas9 protein, a Cas9 or dCas9 protein coding sequence, dgRNA or gRNA molecule, nucleic acid encoding an MS2-transcriptional activator fusion protein, MS2-transcriptional activator fusion protein (such as SEQ ID NO: 18), or combinations thereof.

In some examples, the kit includes a delivery system (e.g., liposome, a particle, an exosome, a microvesicle, a viral vector, or a plasmid), and/or a label (e.g., a peptide or antibody that can be conjugated either directly to an RNP or to a particle containing the RNP to direct cell type specific uptake/enhance endosomal escape/enable blood-brain barrier crossing etc.). In some examples, the kits further include cell culture or growth media, such as media appropriate for growing bacterial, plant, insect, or mammalian cells.

In some examples, such parts of a kit are in separate containers (such as glass or plastic vials).

D. Targeted Gene Activation (TGA) System

Also provided is a targeted gene activation (TGA) system. The system can include a first vector (such as a viral vector, e.g., AAV) that includes a nucleic acid encoding a Cas9 or dCas9 (whose expression can be driven by a promoter) and a second vector (such as a viral vector, e.g., AAV) that includes a gRNA disclosed herein and a nucleic acid encoding an MS2-transcriptional activator fusion protein (such as MS2-p65-HSF1) (whose expression can be driven by a promoter). In some examples, the nucleic acid encoding an MS2-transcriptional activator fusion protein encodes MS2-p65-HSF1, such as a sequence encoding a protein sequence having at least 90%, at least 95%, at least 98%, at least 99%, or 100% sequence identity to SEQ ID NO: 18.

In some examples, the first and first and second vector are viral vectors, such as an adeno-associated viral (AAV) vectors (e.g., an AAV1 vector, AAV2 vector, AAV3 vector, AAV4 vector, AAV5 vector, AAV6 vector, AAV7 vector, AAV8 vector, AAV9 vector, AAV10 vector, AAV11 vector, AAV12 vector, AAV-PHP.B vector, AAV-PHP.eB vector, or AAV-PHP.S vector). In one example, the first and first and second vector are AAV9 vectors. In some examples, the first and first and second vector are AAV8 vectors. In some examples, the AAV vector used has tropism for a specific tissue or cell-type, such as a kidney cell, skeletal muscle cell, or pancreatic cell (examples provided elsewhere herein).

In some examples, the first vector includes a nucleic acid encoding a Cas9 protein, such as a Streptococcus pyogenes Cas9 protein. In some examples, the first vector includes a nucleic acid encoding a Cas9 protein, such as a nucleic acid molecule encoding a protein having at least 90%, at least 95%, at least 98%, at least 99%, or 100% sequence identity to SEQ ID NO: 16, wherein the Cas9 protein has endonuclease activity. In some examples, the first vector includes a nucleic acid encoding a dCas9 protein, such as a dCas9 protein with reduced or no endonuclease activity. In some examples, the first vector includes a nucleic acid encoding a dCas9 protein, such as a nucleic acid molecule encoding a protein having at least 90%, at least 95%, at least 98%, at least 99%, or 100% sequence identity to SEQ ID NO: 17, wherein the dCas9 protein has reduced or endonuclease activity. In some examples, the dCas9 protein encoded by the nucleic acid molecule has a D10A, E762A, D839A, H840A, N854A, N863A, D986A, or combinations thereof, mutation.

In some examples, the first vector includes a nucleic acid encoding a Cas9 or dCas9 protein does not encode a transcriptional activator, such as VP64, P65, MyoD1, HSF1, RTA, SET7/9, or any combination thereof. Thus, in some examples, the Cas9 or dCas9 protein encoded by the first vector is not a Cas9-transcriptional activator fusion protein or a dCas9-transcriptional activator fusion protein.

The second vector includes a gRNA or dgRNA disclosed herein, such as one having at least 90%, at least 95%, at least 98%, at least 99%, or 100% sequence identity to SEQ ID NO: 1-4, 6, 28-31, or 33 and can further include at its 5′-end a sequence of 14 to 30 nt that is complementary to the target nucleic acid. In one example, the gRNA has at least 90%, at least 95%, at least 98%, at least 99%, or 100% sequence identity to SEQ ID NO: 5 or 32 and also includes at the 5′-end, a sequence of 14 or 15 nt that is complementary to a target nucleic acid.

The second vector also includes a nucleic acid encoding an MS2-transcriptional activator fusion protein. MS2-transcriptional activator fusion proteins include an MS2 domain fused directly or indirectly (e.g., via a linker) with a transcriptional activation domain. Exemplary transcriptional activation domains include VP64, p65, MyoD1, HSF1, RTA, SET7/9, or any combination thereof. Exemplary MS2-transcriptional activator fusion proteins are shown in FIG. 8C, and in one example is MS2-p65-HSF1. Thus, in some examples, the nucleic acid encoding an MS2-transcriptional activator fusion protein encodes MS2-p65-HSF1, such as a sequence encoding a protein sequence having at least 90%, at least 95%, at least 98%, at least 99%, or 100% sequence identity to SEQ ID NO: 18.

In some examples, a TGA system allows for multiple genes to be targeted. Thus, in such examples, the TGA system further includes one or more additional gRNAs or dgRNAs, each containing a different targeting sequence than the first gRNA or dgRNA. Multiple additional gRNAs or dgRNAs can be used, each targeting a different gene of interest. Such additional gRNAs or dgRNAs can be on additional vectors, or can also be on the second vector.

### IV. Methods of Targeted Gene Activation

Provided herein are methods of increasing expression (e.g., activating expression) of at least one gene product in vitro or in a subject. The gene product whose expression is increased can be the gene itself (e.g., DNA), an RNA (such as mRNA, miRNA, and non-coding RNA), or protein. When used in vitro, expression can be increased in a cell, such as a eukaryotic or prokaryotic cell, such as a mammalian cells. When used in vivo, expression can be increased in a mammal, such as a mouse (or other veterinary subject) or a human. Methods of using the disclosed gRNAs and TGA systems are also provided. Such methods can be used to increase expression of at least one target gene product in a subject, such as a gene whose expression is decreased in the subject. In some examples, such methods treat a disease in the subject caused by the decreased expression of the target. In some examples, the methods increase expression of the target gene or gene product by at least 10%, at least 20%, at least 25%, at least 50%, at least 60%, at least 70%, at least 75%, at least 80%, at least 90%, at least 95%, at least 100%, at least 200%, at least 300%, at least 400%, or at least 500%.

In some examples, the method is an in vivo method of increasing expression (e.g., activating expression) of at least one gene product in a subject. The method includes administering a therapeutically effective amount of a targeted gene activation (TGA) system to a subject. In some examples, the method is an in vitro method of increasing expression (e.g., activating expression) of at least one gene product in a cell or cell-free system. The method includes contacting an effective amount of a targeted gene activation (TGA) system with the cell or cell-free system. The components of the TGA system infect a cell (e.g., in the subject, such as a cell of the muscle, liver, heart, lung, kidney, spinal cord, or stomach, such as a liver or muscle cell) or express the nucleic acid components of the TGA system, thereby increasing expression of the at least one gene product in the infected cell or cell-free system.

The TGA system is administered in accord with known methods, such as intravenous administration, e.g., as a bolus or by continuous infusion over a period of time, or by intramuscular, intraperitoneal, intracerobrospinal, subcutaneous, intra-articular, intrasynovial, intrathecal, oral, topical, intratumoral, or inhalation routes. The administration may be local or systemic. The TGA system can be administered via any of several routes of administration, including topically, orally, parenterally, intravenously, intra-articularly, intraperitoneally, intramuscularly, subcutaneously, intracavity, transdermally, intrahepatically, intracranially, intratumorally, intraosseously, nebulization/inhalation, or by installation via bronchoscopy. Thus, the compositions are administered in a number of ways depending on whether local or systemic treatment is desired and on the area to be treated.

An effective amount of a nucleic acid molecule or vector disclosed herein can be based, at least in part, on the particular vector used; the individual's size, age, gender; and the size and other characteristics of the proliferating cells. For example, for treatment of a human, at least 10³ viral genomes (vg) per kg of body weight of a viral vector is used, such as at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, at least 10¹⁸, at least 10¹⁹, or at least 10²⁰ vg/kg of body weight, for example, approximately 10³ to 10²⁰, 10⁹ to 10¹⁶, 10¹² to 10¹⁵, or 10¹³ to 10¹⁴ vg/kg of body weight of a viral vector is used.

A nucleic acid or protein, such as a viral vector (e.g., AAV vector), can be administered in a single dose or in multiple doses (e.g., two, three, four, six, or more doses). Multiple doses can be administered concurrently or consecutively (e.g., over a period of days or weeks).

The TGA system used in the method can include (1) a first vector includes a nucleic acid encoding a Cas9 protein or dCas9 protein and (2) a second vector comprising a gRNA or dsgRNA disclosed herein and a nucleic acid encoding an MS2-transcriptional activator fusion protein. In some examples, the first and second vector are adeno-associated viral (AAV) vectors, such as an AAV1 vector, AAV2 vector, AAV3 vector, AAV4 vector, AAV5 vector, AAV6 vector, AAV7 vector, AAV8 vector, AAV9 vector, AAV10 vector, AAV11 vector, AAV12 vector AAV-PHP.B vector, AAV-PHP.eB vector, or AAV-PHP.S vector. In one example, the first and second vector are AAV9 vectors. In some examples, the AAV vector used has tropism for a specific tissue or cell-type, such as a kidney cell, skeletal muscle cell, or pancreatic cell (examples provided elsewhere herein).

When selecting elements for the disclosed TGA system, which allow for gene activation without introducing DNA double strand breaks, either the Cas9 protein used or the gRNA (or both) needs to be a dead form. Thus, in some examples, a dCas9 protein (e.g., SEQ ID NO: 17) is used with a gRNA (such as any of SEQ ID NOS: 1-6 or 28-33+a targeting sequence of about 17-30 nt). In some examples, a Cas9 protein (e.g., SEQ ID NO: 16) is used with a dgRNA (such as any of SEQ ID NOS: 1-6 or 28-33+a targeting sequence of 14 nt or 15 nt). In some examples, a dCas9 protein (e.g., SEQ ID NO: 17) is used with a dgRNA (such as any of SEQ ID NOS: 1-6 or 28-33+a targeting sequence of 14 nt or 15 nt).

In some examples, the first vector includes a nucleic acid encoding a Cas9 protein, such as a Streptococcus pyogenes Cas9 protein. In some examples, the first vector includes a nucleic acid encoding a Cas9 protein, such as a nucleic acid molecule encoding a protein having at least 90%, at least 95%, at least 98%, at least 99%, or 100% sequence identity to SEQ ID NO: 16, wherein the Cas9 protein has endonuclease activity. In some examples, the first vector includes a nucleic acid encoding a dCas9 protein, such as a dCas9 protein with reduced or no endonuclease activity. In some examples, the first vector includes a nucleic acid encoding a dCas9 protein, such as a nucleic acid molecule encoding a protein having at least 90%, at least 95%, at least 98%, at least 99%, or 100% sequence identity to SEQ ID NO: 17, wherein the dCas9 protein has reduced or endonuclease activity. In some examples, the dCas9 protein encoded by the nucleic acid molecule has a D10A, E762A D839A, H840A, N854A, N863A, D986A, or combinations thereof, mutation.

In some examples, the first vector includes a nucleic acid encoding a Cas9 or dCas9 protein does not encode a transcriptional activator, such as VP64, P65, MyoD1, HSF1, RTA, SET7/9, or any combination thereof. Thus, in some examples, the Cas9 or dCas9 protein encoded by the first vector is not a Cas9-transcriptional activator fusion protein or a dCas9-transcriptional activator fusion protein.

The second vector includes a gRNA or dgRNA disclosed herein, such as one having at least 90%, at least 95%, at least 98%, at least 99%, or 100% sequence identity to SEQ ID NO: 1-4, 6, 28-31, or 33, and can further include at its 5′-end a sequence of 14 to 30 nt that is complementary to the target nucleic acid. In one example, the gRNA has at least 90%, at least 95%, at least 98%, at least 99%, or 100% sequence identity to SEQ ID NO: 5 or 32 and also includes, at the 5′-end, a sequence of 14 or 15 nt that is complementary to a target nucleic acid.

The second vector also includes a nucleic acid encoding an MS2-transcriptional activator fusion protein. MS2-transcriptional activator fusion proteins include an MS2 domain fused directly or indirectly (e.g., via a linker) with a transcriptional activation domain. Exemplary transcriptional activation domains include VP64, p65, MyoD1, HSF1, RTA, SET7/9, or any combination thereof. Exemplary MS2-transcriptional activator fusion proteins are shown in FIG. 8C, and, in one example, the MS2-transcriptional activator fusion protein includes MS2-p65-HSF1. Thus, in some examples, the nucleic acid encoding an MS2-transcriptional activator fusion protein encodes MS2-p65-HSF1, such as a sequence encoding a protein sequence having at least 90%, at least 95%, at least 98%, at least 99%, or 100% sequence identity to SEQ ID NO: 18.

In some examples, multiple genes are targeted, for example, in the same subject or same cell. Thus, in such examples, the TGA system further includes one or more additional gRNAs or dgRNAs, each containing a different targeting sequence than the first gRNA or dgRNA. Multiple additional gRNAs or dgRNAs can be used, each targeting a different gene of interest. Such additional gRNAs or dgRNA can be on additional vectors or can also be on the second vector.

In one example, the Cas9, dCas9, and/or MS2-transcriptional activator fusion protein is expressed in a recombinant cell, such as E. coli, and purified. The resulting purified Cas9, dCas9, and/or MS2-transcriptional activator fusion protein, along with one or more gRNAs or dgRNAs specific for one or more target sequences, is then introduced into a cell or organism where one or more genes can be upregulated. In some examples, the Cas9, dCas9, and/or MS2-transcriptional activator fusion protein and guide nucleic acid molecule are introduced as separate components into the cell/organism. In other examples, the purified Cas9, dCas9, and/or MS2-transcriptional activator fusion is complexed with the guide nucleic acid (e.g., gRNA or dgRNA), and this ribonucleoprotein (RNP) complex is introduced into target cells (e.g., using transfection or injection). In some examples, the Cas9, dCas9, and/or MS2-transcriptional activator fusion protein and guide molecule are injected into an embryo (such as a human, mouse, zebrafish, or Xenopus embryo). Once the Cas9 or dCas9 protein, MS2-transcriptional activator fusion protein, and guide nucleic acid molecule are in the cell, expression of one or more target nucleic acid molecules can be activated.

One or more nucleic acid molecules can be targeted by the disclosed methods, such as at least 1, at least 2, at least 3, at least 4, or at least 5 different nucleic acid molecules in a cell or organism, such as 1, 2, 3, 4, 5, 6, 7, 8, 9, or 10 different nucleic acid molecules. In some examples, the disclosed methods are used to treat or prevent a disease associated with no or reduced expression of one or more genes (e.g., a reduction of at least 10%, at least 20%, at least 30%, at least 40%, at least 50%, at least 75%, at least 80%, at least 90%, at least 95%, at least 99%, or 100% reduction). In one example, the target is associated with a disease such as type I diabetes, Duchenne muscular dystrophy, or acute kidney disease. In some examples, the disease is of the liver, muscle, pancreas, or kidney. In some examples, the disease is a disease of the liver, such as Alagille Syndrome; alpha-1 antitrypsin deficiency (alpha-1); biliary atresia; cirrhosis; galactosemia; Gilbert syndrome; hemochromatosis; Lysosomal acid lipase deficiency (LAL-D); non-alcoholic fatty liver disease (NAFLD); primary biliary cholangitis (PBC); primary sclerosing cholangitis (PSC); type I glycogen storage disease (GSD I); and Wilson disease. In some examples, the gene or gene product targeted (e.g., is activated) is one or more of Fst, Pdx1, klotho, utrophin, interleukin 10, insulin 1, insulin 2, Pcsk1, or Six2.

Specific examples of diseases that can be treated, along with genes that can be targeted (e.g., activated) with the disclosed methods, are shown in the table below. Additional examples can be found in US Publication no. 2016/0355797 (herein incorporated by reference in its entirety).

| Disease | Targets | References |
|---|---|---|
| Acute Kidney Disease | IL-10 | Liao (2017) |
| | Klotho | Liao (2017) |
| Aging | TERT | Bernardes (2012) |
| | Oct4, Sox2, | Ocampo (2016) |
| | Klf4, c-Myc | |
| Alzheimer's disease | Neprilysin, | EI-Amouri (2008), |
| | NGF, BDNF | Nilsson (2010) |
| Cancer | Bax | Falke (2003) |
| | Notch3 | Rahman (2012) |
| Degenerative motor | HGF | Sun (2002), |
| neuron diseases (ex. | CNTF | Schaller (2017) |
| ALS, SMA) | Artemin | |
| Diabetes | Pdx1 | Liao (2017) |
| | Pdx1, MafA | Xiao (2018) |
| | Pdx1, Ngn3, | Zhou (2008), |
| | MafA | Cavelti-Weder (2017) |
| Liver fibrosis | Foxa3, Gata4, | Song (2016) |
| | HNF4a, HNF4a | |
| Muscular Dystrophy | Utrophin | Liao (2017) |
| | Follistatin | Liao (2017) |
| | Klotho | Liao (2017) |
| Myocardial disorders | Tbx18 | Kapoor (2013) |
| Sickle cell disease | Gamma-Globin | Gräslund (2005) |

Bernardes et al., EMBO Mol Med. 2012 August; 4(8):691-704
Cavelti-Weder et al., Curr Protoc Stem Cell Biol. 2017 February; 40:4A.10.1-4A.10.12
El-Amouri et al., Am J Pathol. 2008 May; 172(5):1342-54.
Falke et al., Nucleic Acids Res. 2003 Feb. 1; 31(3):e10
Gräslund et al., J Biol Chem. 2005 Feb. 4; 280(5):3707-14.
Kapoor et al., Nat Biotechnol. 2013 January; 31(1):54-62.
Liao et al., Cell. 2017 Dec. 14; 171(7):1495-1507.
Ocampo et al., Cell. 2016 Dec. 15; 167(7):1719-1733
Per Nilsson et al., J. Cell Mol. Med., 2010 April; 14(4): 741-757.
Rahman et al., Am J Clin Pathol. 2012 October; 138(4):535-44.
Schaller et al., Proc Natl Acad Sci USA. 2017 Mar. 21; 114(12):E2486-E2493.
Song et al., Cell Stem Cell. 2016 Jun. 2; 18(6):797-808.
Sun et al., J Neurosci. 2002 Aug. 1; 22(15):6537-48.
Xiao et al., Cell Stem Cell. 2018 Jan. 4; 22(1):78-90
Zhou et al., Nature. 2008 Oct. 2; 455(7213):627-32.

Specific examples of additional genes that can be targeted (e.g., activated) with the disclosed methods, are shown in the table below. In certain embodiments, the targeting sequence is complementary to a sequence at least within 10 nt, 25 nt, 50 nt, 60 nt, 70 nt, 80 nt, 90 nt, 100 nt, 110 nt, 120 nt, 130 nt, 140 nt, 150 nt, 175 nt, 200 nt, 300 nt, 400 nt, or 500 nt of the transcriptional start site.

| Gene | NCBI Ref. No. | Exemplary Disease |
|---|---|---|
| TTR | S63185.1 | Amyloid neuropathy |
| APOA1 | NG_012021.1 | Amyloidosis |
| APP | NG_007376.1 | Amyloidosis |
| GSN | NG_012872.1 | Amyloidosis |
| FGA | NG_008832.1 | Amyloidosis |
| LYZ | NG_008195.1 | Amyloidosis |
| KRT18 | NG_008351.1 | Cirrhosis |
| KRT8 | NG_008402.2 | Cirrhosis |
| CIRH1A | NG_008278.1 | Cirrhosis |
| CFTR | NG_016465.4 | Cystic fibrosis |
| MRP7 | AL359813.23 | Cystic fibrosis |
| SLC2A2 | NG_008108.1 | Glycogen storage diseases |
| G6PC | NG_011808.1 | Glycogen storage diseases |
| G6PT1 | NG_013331.1 | Glycogen storage diseases |
| GAA | NG_009822.1 | Glycogen storage diseases |
| LAMP2 | NG_007995.1 | Glycogen storage diseases |
| AGL | NG_012865.1 | Glycogen storage diseases |
| GBE1 | NG_011810.1 | Glycogen storage diseases |
| GYS2 | NG_016167.1 | Glycogen storage diseases |
| PYGL | NG_012796.1 | Glycogen storage diseases |
| PFKM | NG_016199.2 | Glycogen storage diseases |
| HNF1A | NG_011731.2 | Hepatic adenoma, 142331 |
| SCO1 | NG_008228.2 | Hepatic failure, early onset, and |
| | | neurologic disorder |
| LIPC | NG_011465.1 | Hepatic lipase deficiency |
| CTNNB1 | NG_013302.2 | Hepatoblastoma |
| PDGFRL | NG_023332.1 | Hepatoblastoma |
| AXIN1 | NG_012267.1 | Hepatoblastoma |
| TP53 | NG_017013.2 | Hepatoblastoma |
| IGF2R | NG_011785.3 | Hepatoblastoma |
| MET | NG_008996.1 | Hepatoblastoma |
| CASP8 | NG_007497.1 | Hepatoblastoma |
| UMOD | NG_008151.1 | Medullary cystic kidney disease |
| HNF1B | NG_013019.2 | Medullary cystic kidney disease |
| PAH | NG_008690.2 | Phenylketonuria |
| QDPR | NG_008763.1 | Phenylketonuria |
| PTS | NG_008743.1 | Phenylketonuria |
| PKHD1 | NG_008753.1 | Polycystic kidney and hepatic |
| | | disease |
| PKD1 | NG_008617.1 | Polycystic kidney and hepatic |
| | | disease |
| PKD2 | NG_008604.1 | Polycystic kidney and hepatic |
| | | disease |
| PRKCSH | NG_009300.1 | Polycystic kidney and hepatic |
| | | disease |
| SEC63 | NG_008270.1 | Polycystic kidney and hepatic |
| | | disease |
| PCSK9 | NG_009061.1 | Liver Disease |
| Hmgcr | NG_011449.1 | Liver Disease |
| SERPINA1 | NG_008290.1 | Liver Disease |
| ApoB | NG_011793.1 | Liver Disease |
| HNF4A | NG_009818.1 | Liver fibrosis/cirrhosis |
| FOXA2 | AF147787.1 | Liver fibrosis/cirrhosis |
| OCT4 | Gene ID: 5460 | Liver fibrosis/cirrhosis |
| FOXA1 | AF147787.1 | Liver fibrosis/cirrhosis |
| FOXA3 | GeneID: 3171 | Liver fibrosis/cirrhosis |
| HNF6 | GeneID: 3175 | Liver fibrosis/cirrhosis |
| GATA4 | NG_008177.2 | Liver fibrosis/cirrhosis |
| HLF | NG_046944.1 | Liver fibrosis/cirrhosis |
| CEBPA | NG_012022.1 | Liver fibrosis/cirrhosis |
| PROX1 | Gene ID: 5629 | Liver fibrosis/cirrhosis |
| AMT | NG_015986.1 | Liver Disease, fibrosis/cirrhosis |
| ADA | NG_007385.1 | Liver Disease, fibrosis/cirrhosis |
| PPOX | NG_012877.2 | Liver Disease, fibrosis/cirrhosis |
| UROD | NG_007122.2 | Liver Disease, fibrosis/cirrhosis |
| HMBS | NG_008093.1 | Liver Disease, fibrosis/cirrhosis |
| ACADVL | NG_007975.1 | Liver Disease, fibrosis/cirrhosis |
| PC | NG_008319.1 | Liver Disease, fibrosis/cirrhosis |
| IVD | NG_011986.2 | Liver Disease, fibrosis/cirrhosis |
| APOA5 | NG_015894.1 | Liver Disease, fibrosis/cirrhosis |
| GALT | NG_009029.2 | Liver Disease, fibrosis/cirrhosis |
| LDLRAP1 | NG_008932.1 | Liver Disease, fibrosis/cirrhosis |
| GCK | NG_008847.2 | Liver Disease, fibrosis/cirrhosis |
| POGLUT1 | NG_034115.1 | Liver Disease, fibrosis/cirrhosis |
| PIK3R1 | NG_012849.2 | Liver Disease, fibrosis/cirrhosis |
| TRIB1 | FJ515869.1 | Liver Disease, fibrosis/cirrhosis |
| TGFB1 | NG_013364.1 | Liver Disease, fibrosis/cirrhosis |
| HAMP | NG_011563.1 | Liver Disease, fibrosis/cirrhosis |
| THPO | NG_012136.1 | Liver Disease, fibrosis/cirrhosis |
| PNPLA3 | NG_008631.1 | Liver Disease, fibrosis/cirrhosis |
| ATP7B | NG_008806.1 | Liver Disease, fibrosis/cirrhosis |
| FAH | NG_012833.1 | Liver Disease, fibrosis/cirrhosis |
| ASL | NG_009288.1 | Liver Disease, fibrosis/cirrhosis |
| HFE | NG_008720.2 | Liver Disease, fibrosis/cirrhosis |
| ALMS1 | NG_011690.1 | Liver Disease, fibrosis/cirrhosis |
| PPARD | NG_012345.1 | Liver Disease, fibrosis/cirrhosis |
| IL6 | NG_011640.1 | Liver Disease, fibrosis/cirrhosis |
| HSD3B7 | NG_012346.1 | Liver Disease, fibrosis/cirrhosis |
| CERS2 | Gene ID: 29956 | Liver Disease, fibrosis/cirrhosis |
| NCOA5 | Gene ID: 57727 | Liver Disease, fibrosis/cirrhosis |
| HEX | Gene ID: 3087 | Liver Disease, fibrosis/cirrhosis |
| HNF6 | Gene ID: 378468 | Liver Disease, fibrosis/cirrhosis |
| SOX17 | NG_028171.1 | Liver Disease, fibrosis/cirrhosis |

### V. Reporters

Disclosed herein are systems, kits, and methods for measuring gene activation, such as where Cas9 (e.g., Cas9 or dead Cas9, dCas9) is expressed or with a Cas9 expression step. The systems, kits, and methods for measuring gene activation herein can be used for any assay, such as assaying the efficiency of a gene activation system (e.g., a TGA system disclosed herein) and/or isolating or sorting cells (e.g., cells with gene activation or cells without gene activation).

Provided herein are systems and kits for measuring gene activation when Cas9 is expressed. In some examples, the systems and kits include at least one gene activation vector and at least one reporter vector. Cas9, including Cas9 or dCas9, can be expressed constitutively or inducibly as well as endogenously or exogenously using any method, kit, system, or composition, including the methods, kits, systems, and compositions disclosed herein, such as using a vector (e.g., a viral vector, such as an AAV vector) that encodes Cas9 (e.g., Cas9 or dCas9). In some examples, the at least one gene activation vector includes a gRNA (e.g., dgRNA) and at least one transcriptional activator protein. In some examples, the at least one reporter vector includes a target sequence of the gRNA and at least one reporter protein, in which the reporter protein is positioned downstream of the target sequence.

Methods of measuring gene activation in a subject (e.g., a mammalian subject, such as a mouse or human) are also provided. In some examples, the methods include expressing Cas9 (e.g., Cas9 or dCas9). Cas9, including Cas9 or dCas9, can be expressed constitutively or inducibly as well as endogenously or exogenously using any method, kit, system, or composition, including the methods, kits, systems, and compositions disclosed herein, such as using a vector (e.g., a viral vector, such as an adeno-associated viral (AAV) vector) that encodes Cas9 (e.g., Cas9 or dCas9). In some examples, the methods include injecting the subject with at least one gene activation vector and at least one reporter vector. Any injection method can be used, including subcutaneous, intramuscular, intravenous, intraperitoneal, intracardiac, intraarticular, and/or intracavernous injection of any amount of the at least one gene activation vector and at least one reporter vector (e.g., an effective amount of a vector, such as described herein). In some examples, the at least one gene activation vector includes a guide ribonucleic acid (gRNA) and at least one transcriptional activator protein. In some examples, the at least one reporter vector includes a target sequence of the gRNA and at least one reporter protein, in which the reporter protein is positioned downstream of the target sequence.

In the systems, kits, or methods described herein, the vector of the at least one gene activation vector or the at least one reporter vector can be any vector, such as any vector described herein. In some examples, the vector is a viral vector or plasmid (e.g., retrovirus, lentivirus, adenovirus, adeno-associated virus, or herpes simplex virus). In specific examples, the vector is an AAV vector (e.g., an AAV9 vector). In some examples, the AAV vector has tropism for a specific tissue or cell-type. In some examples, the guide nucleic acid molecule is operably linked to a promoter or expression control element (examples of which are provided elsewhere in this application). In specific examples, the promoter is a minimal promoter, such as cytomegalovirus (CMV), human b-actin (hACTB), human elongation factor-1a (hEF-1a), and cytomegalovirus early enhancer/chicken b-actin (CAG) promoters (e.g., the promoters described in Papadakis et al., Current Gene Therapy, 4:89-113, 2004; Damdindorj et al., PLoS ONE 9(8):e106472, 2014, both of which are incorporated herein by reference). The vectors can include other elements, such as a gene encoding a selectable marker, such as an antibiotic, such as puromycin or hygromycin, or a detectable marker, such as GFP, another fluorophore, or a luciferase protein. Such vectors can include naturally occurring or non-naturally occurring nucleotides or ribonucleotides. Such vectors can be used in the methods, compositions, and kits provided herein.

In the systems, kits, or methods described herein, the at least one reporter vector can include at least one reporter protein that is positioned downstream of a target sequence. Any type of reporter protein can be used, such as a fluorescent protein, a bioluminescent protein, or any combination thereof. Exemplary reporter proteins include infrared-fluorescent proteins (IFPs), mRFP1, mCherry, mOrange, DsRed, dTomato (or tdTomato), mKO, tagRFP, EGFP, mEGFP, mOrange2, maple, tagRFP-T, firefly luciferase, renilla luciferase, and click beetle luciferase (e.g., US Pat. Pub. No. 2010/0122355, incorporated herein by reference). In some examples, the at least one reporter protein can include at least about 1, 2, 3, 4, or 5 or 1-2, 1-3, or 1-5 or about 2 reporter proteins. In specific examples, the at least one reporter protein includes luciferase, mCherry, dTomato, or any combination thereof (e.g., a luciferase and mCherry combination or a luciferase and dTomato combination). The target sequence can be any target sequence of interest that is complementary to the gRNA of the gene activation vector (e.g., a target sequence that is an endogenous gene of the subject or a target sequence that is not an endogenous gene of the subject).

In the systems, kits, or methods described herein, the at least one gene activation vector includes a guide ribonucleic acid (gRNA) and at least one transcriptional activator protein. gRNA sequences are described herein. Any gRNA sequence can be used (e.g., dgRNA). Transcriptional activator proteins are described herein. Any transcriptional activator protein can be used, such as VP64, p65, MyoD1, HSF1, RTA, SET7/9, or any combination thereof. In specific examples, the at least one transcriptional protein includes P65 and HSF1.

### EXAMPLES

The examples herein describe a combination of co-transcriptional activators and sgRNAs that fit within a single AAV vector and induce high levels of target gene activation (TGA). Injection of these single AAVs (AAV-gRNA) into Cas9-expressing mice (Platt et al., 2014) produced efficient TGA and clear phenotypes, thus expanding the utility of Cas9-mice for gain-of-function studies in vivo. Next, the examples describe using this system to ameliorate disease phenotypes (namely, acute kidney injury, type 1 diabetes, and the mdx model of Duchenne muscular dystrophy) by introducing Cas9 transgenes into these disease models. Finally, the examples describe generating a dual-AAV system and demonstrate that co-injection of AAV-Cas9 with an AAV-gRNA that targeted utrophin can ameliorate muscular dystrophy symptoms of mdx mice. In summary, we have developed an in vivo CRISPR/Cas9 TGA system for activating the expression of endogenous genes. This system can induce epigenetic remodeling of targeted loci by recruiting the transcriptional machinery (“trans-epigenetic modulation”) and can be used to treat a wide range of human diseases and injuries.

### Example 1

This example describes the materials and methods for Examples 1-9.

### Experimental Model and Subject Details

All animal procedures were performed according to NIH guidelines and approved by the Committee on Animal Care at the SALK® Institute.

### Mice

ICR, C57BL/6, Rosa26-Cas9 knockin (Gt(ROSA)26Sortm1.1(CAG-Cas9*, -EGFP)Fezh, Stock No. 024858), and Dmdmdx (C57BL/10ScSn-Dmdmdx/J, Stock No. 001801) mice were purchased from the Jackson laboratory. The mice were housed in a 12-hour light/dark cycle (light between 06:00 and 18:00) in a temperature-controlled room (22±1° C.) with free access to water and food. All procedures were performed in accordance with protocols approved by the IACUC and Animal Resources Department of the Salk Institute for Biological Studies. The ages of mice are indicated in the BRIEF DESCRIPTION OF THE DRAWINGS or the figure panel. Both female and male mice were used for behavioral experiments, no notable sex-dependent differences were found in our analyses. For beta-cell ablation experiments, male mice were randomly assigned to experimental and control groups.

### Cell Lines and Cell Culture

The HEK 293A cell line was purchased from INVITROGEN® (Carlsbad, Calif.) and maintained in DMEM medium containing 10% fetal bovine serum (FBS), 2 mM glutamine, 1% non-essential amino acids, and 1% penicillin-streptomycin. Neuro-2a (N2a) cells were originally from SIGMA-ALDRICH® and cultured with the same medium. Cas9 mouse embryonic stem cell (Cas9 mESCs) lines were derived from blastocysts of homozygous Rosa26-Cas9 knockin mice using previously described procedures (Czechanski et al., 2014). Cells were then maintained in N2B272ILIF media on Matrigel (CULTREX®)-coated plates. The female Cas9 mESC cell line was used in this study. This cell line was authenticated via morphology, PCR based genotyping, and sequencing.

### Method Details

### Plasmid Design and Construction

The luciferase reporter (tLuc) was constructed by replacing mCherry with luciferase in the M-tdTom-SP-gT1 plasmid (Addgene 48677)(Esvelt et al., 2013) and then sub-cloning this construct into the AAV backbone construct, as AAV-tLuc. The AAV-tLuc-mCherry reporter was constructed by inserting a 2A-mCheery fragment into AAV-tLuc. The U6-dgRNA-CAG-MPH plasmid was constructed by combining U6-MS2gRNA from the plasmid sgRNA(MS2)_cloning_backbone (Addgene 61424) and the MPH transactivation domain from the plasmid lenti_MS2-P65-HSF1_Hygro (Addgene 61426) under the control of a CAG promoter. U6-dgRNA-CAG-MPH was further sub-cloned into the AAV backbone to make AAV-U6-dgRNA-CAG-MPH. Either 20-bp or 14-bp spacers of gRNAs (Table S1) were inserted into the plasmids with gRNA backbones at either the BsmBI or SapI site. The mock-gRNA target sequence was synthesized as described (Liao et al., 2015). To generate different MS2-fused transcriptional activator constructs, VP64 and Rta were amplified from the SP-dCas9-VPR plasmid (Addgene 63798), and P65 was amplified from the MS2-P65-HSF1_GFP plasmid (Addgene 61423), all of which were subsequently sub-cloned into a pCAG-containing plasmid under the order described in FIGS. 8A-8F. AAV-nEF-Cas9 was described previously (Suzuki et al., 2016). AAV-CMVc-Cas9 was constructed by replacing the Mecp2 promoter of PX551 (Swiech et al., 2015) with a core CMV promoter. AAV-nEF-dCas9 was constructed by replacing the Cas9 of pAAV-nEF-Cas9 with dCas9 coding sequence.

### Transfection of In Vitro Cultured Cells

LIPOFECTAMINE® 2000 or 3000 (THERMOFISHER®) was used to transfect HEK293 cells, N2a, and Cas9 mESCs. Transfection complexes were prepared following the manufacturer's instructions.

### Luciferase-Based Reporter Assay

After harvesting luciferase-expressing cells by TRYPLE® Express (LIFE TECHNOLOGIES®), suspended cells were transferred to 96-well plates, and reagents from DUAL-GLO® Luciferase Assay System (PROMEGA®) were applied. The luminescent signal was quantified using a SYNERGY® H1 Hybrid Reader (BIOTEK®) with triplicated wells per sample.

### AAV and Lentivirus Production

AAV2/9 (AAV2 inverted terminal repeat (ITR) vectors pseudo-typed with AAV9 capsid) viral particles were generated by or following the procedures of the Gene Transfer Targeting and Therapeutics Core at the SALK® Institute for Biological Studies. Lentiviral vectors were packed as described, and the vesicular stomatitis virus Env glycoprotein (VSV-G) was used (Liao et al., 2015).

In vivo Muscle Electroporations

Wild type or Cas9-expressing mice were anaesthetized with intraperitoneal injection of ketamine (100 mg/kg) and xylazine (10 mg/kg). A small portion of the quadriceps muscle was surgically exposed in the hind limb. A plasmid DNA mixture (25 μg of each plasmid in 50 μl TE) was injected into the muscle using a 29-gauge insulin syringe. One minute following plasmid DNA injection, a pair of electrodes was inserted into the muscle to a depth of 5 mm to encompass the DNA injection site. Muscle was electroporated using an Electro Square Porator T820 (BTX Harvard Apparatus). Electrical stimulation was delivered in twenty pulses at 100 V for 20 ms. After electroporation, the open sites were closed by stitches, and the mice were allowed to recover from the anesthesia on a 37° C. warm pad.

### Intramuscular (IM) AAV Injection

Newborn (P2.5) mice were used for intramuscular injections. The AAV mixtures (AAV9-dgRNA (1×1011 GC); AAV9-tLuc reporter (1×1010 GC)) were injected into the tibialis anterior (TA) and quadriceps femoris (QA) muscles under anesthesia. For 3-week-old mice, the mice were anaesthetized with intraperitoneal injection of ketamine (100 mg/kg) and xylazine (10 mg/kg). A small portion of the quadriceps muscle was surgically exposed in the hind limb. The AAVs were injected into the TA muscle and/or the QF muscle using a 33 gauge HAMILTON® syringe. After AAV injection, the skin was closed by stitches, and the mice recovered on a 37° C. warm pad.

### Facial Vein AAV Injection

Newborn (P0.5) mice were used for facial vein injection as described (Gombash Lampe et al., 2014). The AAV mixtures (AAV9-dgRNA (1×1011 GC); AAV9-tLuc reporter (1×1010 GC)) were injected via the temporal vein of the P0.5 mice.

### Intra-Cerebral AAV Injection

Neonatal mice were used for intra-cerebral injections as described (Kim et al., 2014). The AAV mixtures (AAV9-dgRNA (5×1010 GC); AAV9-tLuc reporter (1×1010 GC)) were injected intracranially into neonatal mice.

### Tail Vein AAV Injection

C57BL/6 mice and Cas9 mice (males and females, 8 to 12 weeks old) received tail vein injections of AAV (AAV9-dgRNA (3.5×1012 GC)). Liver tissues and serum samples were collected 13 days after tail vein injections. Collected liver samples were used for qRT-PCR or fixed in 4% Paraformaldehyde (PFA) and then embedded in OCT compound after a PBS wash and quickly frozen in ethanol. Cryostat sections (10 μm) were labeled for insulin, HNF3B, PDX1, or SIX2.

### Bioluminescence Imaging (BLI)

Mice were examined at each time point after electroporation or AAV infection for BLI analysis using an IVIS® Kinetic 2200 (CALIPER LIFE SCIENCES®, now PERKINELMER®). Mice were injected intraperitoneally with 150 mg/kg D-Luciferin (SYNLAB®), anesthetized with isoflurane, and then images were captured within 10 minutes of D-Luciferin injection.

### Cisplatin-Induced Acute Kidney Injury Mouse Model

Cas9 mice (males and females, 8 to 12 weeks old) received an intraperitoneal injection of 15 mg/kg cisplatin (TOCRIS BIOSCIENCE®, Ellisville, Mo.) 8 days after tail vein injection of AAV. Kidney tissues and blood serum samples were collected 4 days after cisplatin administration. Blood serum was assayed for blood urea nitrogen (BUN) and serum creatinine (S-Cre) levels using commercially available assays (QUANTICHROM® Urea Assay Kit and QUAINTCHROM® Creatinine Assay Kit; BioAssay Systems, Hayward, Calif.) as renal function parameters. Collected kidney samples were fixed in 4% paraformaldehyde (PFA), embedded in OCT compound (Sakura Tissue-Tek®) after PBS wash, and quickly frozen in ethanol. Cryostat sections (10 μm) were stained with either hematoxylin and eosin (H&E) or periodic acid-Schiff's reagent (PAS). Tubular necrosis, urinary casts, tubular dilation, and tubular borders were assessed in non-overlapping fields (high power field) as described (Imberti et al., 2015; Li et al., 2016).

### Beta Cell Ablation

Induction of diabetes by high-dose streptozocin (STZ) treatment was performed in Cas9 male mice that were 2-4 months old. A single STZ dose (160 mg/kg) in 0.1 M sodium citrate buffer (pH 4.5) was injected intraperitoneally after the mice were fasted for 5 hours. Forty-eight hours later, the mice were randomly grouped for injection of AAV9 with dgMock or dgPdx1 through tail vein. The blood glucose levels were measured every other day with a ONETOUCH® ULTRA® 2 glucometer (ONETOUCH®) using blood from the tail vein. The mice were sacrificed at indicated times, and livers were dissected and processed for histological analysis.

### Immunohistochemistry

Tissues were harvested after transcardial perfusion using ice-cold PBS, followed by ice-cold 4% paraformaldehyde in phosphate buffer for 15 min. Tissues were dissected out and postfixed in 4% paraformaldehyde overnight at 4° C. and cryoprotected in 30% sucrose overnight at 4° C. and embedded in OCT (Sakura TISSUE-TEK®) and frozen on dry ice. For muscle, after tissue dissection, muscle was frozen in isopentane in liquid nitrogen. Serial sections at 10 μm were made with a cryostat and collected on SUPERFROST® Plus slides (FISHER SCIENTIFIC®) and stored at −80° C. until use. Immunohistochemistry was performed as follows: sections were washed with PBS for 5 min 3 times, incubated with a blocking solution (PBS containing 2% donkey serum (or 5% BSA) and 0.3% Triton X-100) for 1 h, incubated with primary antibodies diluted in the blocking solution overnight at 4° C., washed with PBST (0.2% Tween 20 in PBS) for 10 min 3 times, and incubated with secondary antibodies conjugated to ALEXA FLUOR® 488, ALEXA FLUOR® 546, or ALEXA FLUOR® 647 (THERMO FISHER®) for 1 h at room temperature. After washing, the sections were mounted with mounting medium (DAPI FLUOROMOUNT-G®, SouthernBiotech). For muscle staining, an antigen retrieval process was carried out by heating the sections for 20 min at 70° C. in HistoVT One solution (Nacalai tesque) and washed two times with PBS. The primary antibodies used in this study were anti-Laminin, 1:100 (L9393, Sigma); anti-Pdx1, 1:100 (ab47267, ABCAM®); anti-Insulin, 1:100 (ab7842, ABCAM®); anti-Six2, 1:200 (11562-1-AP, PROTEINTECH®); anti-Hnf-3β, 1:100 (sc-101060, Santa Cruz); and anti-Utrophin, 1:50 (sc-15377, Santa Cruz).

### RNA Analysis

Total RNA was extracted from cells and tissue samples using either TRIZOL® (INVITROGEN®) or RNeasy® Kit (QIAGEN®) followed by cDNA synthesis using ISCRIPT® Reverse Transcription Supermix for RT-PCR (BIO-RAD®). qPCR was performed using SSOADVANCED® SYBR® Green Supermix and analyzed using a CFX384 Real-Time system (BIO-RAD®). All analyses were normalized based on amplification of human or mouse Gapdh. Primer sequences for qPCR are listed in Table S2.

### Enzyme-Linked Immunosorbent Assay (ELISA)

Mouse sera was subjected to ELISA assay following the standard protocol (Mouse Klotho ELISA kit, CUSABIO®; Mouse IL-10 ELISA kit, AFFYMETRIX® EBIOSCIENCE®; Mouse Insulin ELISA kit, ALPCO®). ELISA assays were performed in duplicate at three separate times, and the data are expressed as mean±SD.

### Wire Hang Test

A single 2-mm diameter wire from a metal hanger was used in this test. The vertical distance between the wire and fall point was set at 37 cm. The mouse was lifted by the tail and allowed to grasp the middle of a metal wire with its forepaws. The hanging latency was recorded until each mouse fell. Two measurements were taken per mouse. The longest hanging time was used for statistical analysis.

### Grip Strength Test

Fore and hind limb grip strengths were assessed using a grip strength meter (CHATILLON® Force Measurement Systems, Largo, Fla.). Mice were lifted by the tail, and the forepaws and backpaws were each allowed to grasp onto the steel grid attached to the apparatus. The mouse was then gently pulled across the steel grid until its grip was released. Mice were tested 5 times, and the three highest measured values were averaged to calculate grip strength.

### Chromatin Immunoprecipitation (ChIP)—Quantitative PCR

ChIP procedures were modified from a previous report (Hatanaka et al., 2010). Tissues were fixed in PBS containing 0.5% formaldehyde for 15 min. Glycine was added to a final concentration of 0.125 M, and the incubation was continued for an additional 15 min. After washing the samples with ice-cold PBS, the samples were homogenized in 1 mL of ice-cold homogenize buffer (5 mM PIPES [pH 8.0], 85 mM KCl, 0.5% NP-40, and protease inhibitors cocktail) and centrifuged (18,000×g, 4° C., 5 min). The pellets were suspended in nucleus lysis buffer (50 mM Tris-HCl [pH 8.0], 10 mM EDTA, 1% SDS, protease inhibitors) and sonicated 15 times for 10 s each time at intervals of 50 s with a Sonic Dismembrator 550 (FISHER SCIENTIFIC®). The samples were centrifuged at 18,000 g at 4° C. for 5 min. Supernatants were diluted 10-fold in ChIP dilution buffer (50 mM Tris-HCl [pH 8.0], 167 mM NaCl, 1.1% Triton X-100, 0.11% sodium deoxycholate, protease inhibitor). Nonspecific background was removed by incubating samples with a fish sperm DNA/protein A-agarose slurry at 4° C. for 2 h with rotation. The samples were centrifuged at 1,000 g at 4° C. for 2 min, and a 0.1 volume of the recovered supernatants was stored as an input sample, whereas the rest was incubated overnight with 2 μg of indicated antibodies at 4° C. with rotation. The immunocomplexes were collected with 50 μl of a fish sperm DNA/protein A/G-agarose (sc-2003, Santa Cruz) at 4° C. for 3 h with rotation. The beads were sequentially washed with the following buffers: radioimmunoprecipitation assay (RIPA) buffer-150 mM NaCl, RIPA buffer-500 mM NaCl, and LiCl wash solution. Finally, the beads were washed twice with 10 mM Tris-HCl (pH 8.0) and 1 mM EDTA. The immunocomplexes were then eluted by the addition of 200 μl of ChIP direct elution buffer (10 mM Tris-HCl [pH 8.0], 300 mM NaCl, 5 mM EDTA, 0.5% SDS) and rotated for 15 min at room temperature and incubated for 4 h at 65° C. The DNA was recovered by phenol-chloroform-isoamyl alcohol (25:24:1) extraction and ethanol precipitation. H3K4me3 (ab8580, ABCAM®), H3K27ac (MA309B, Takara), and IgG-bound DNA were used for quantitative real-time PCR (qRT-PCR). The primers were designed as Table S3.

### SURVEYOR® Assay

The indel frequency was analyzed by surveyor assay (IDT®). Briefly, samples were collected to extract genomic DNA by DNEASY® Blood & Tissue kit (QIAGEN®). The Il-10 or Pdx1 locus was amplified by PCR from 100 ng of genomic DNA using LA TAQ® Hot Start polymerase (TaKaRa) and Il-10 primers (forward: 5′-ccagttctttagcgcttacaatgc-3′ and reverse: 5′-gcagctctaggagcatgtgg-3′) or Pdx1 primers (forward: 5′-aagctcattgggagcggttttg-3′ and reverse: 5′-gtccggaggacttccctgc-3′) in a 20 μl reaction. The PCR product (200 ng) was then denatured and slowly re-annealed using a step-wise gradient temperature program in a T100 thermocyler (BIO-RAD®), followed the protocol adapted from previous publications (Sanjana et al., 2012).

### DNA Library Preparation and Deep Sequencing

Il-10 primers for the SURVEYOR® assay were used for the first round of amplifications in the nested-PCR procedure with limited PCR cycles using 100 ng of genomic DNA from cultured cells or tissues. This PCR product was used for the second round of amplification in the nested-PCR procedure using primer pairs with deep sequencing adaptor (mIl10-adapter-F1: 5′-ACACTCTTTCCCTACACGACGCTCTTCCGATCTcatggtttagaagagggagga-3′ and mIL10-adapter-R1: 5′-GACTGGAGTTCAGACGTGTGCTCTTCCGATCTgagcaggcagcatagcagt-3′). The nested PCR product was purified using the QlAquick PCR Purification Kit (QIAGEN®) for DNA library preparation. NEBNEXT® ULTRA® DNA Library Preparation kit was used to prepare the sequencing library (ILLUMINA®, San Diego, Calif., USA). Adapter-ligated DNA was indexed and enriched by limited cycle PCR. The DNA library was validated using TAPESTATION® (AGILENT® Technologies, Palo Alto, Calif., USA) and was quantified using a QUBIT® 2.0 Fluorometer. The DNA library was quantified by real time PCR (APPLIED BIOSYSTEMS®, Carlsbad, Calif., USA). The DNA library was loaded onto an ILLUMINA® MISEQ® instrument (ILLUMINA®, San Diego, Calif., USA). Sequencing was performed using a 2×150 paired-end (PE) configuration by GENEWIZ®, Inc. (South Plainfield, N.J., USA). The MISEQ® Control Software (MCS) on the MISEQ® instrument conducted image analysis and base calling. The raw sequencing reads were quality and adapter trimmed using Trimmomatic-0.36. The reads were aligned to the target gene reference genome using bwa-0.7.12. The variants were called for each sample using mpileup within samtools-1.3.1 followed by VarScan-2.3.9. At least 50,000 reads per sample was analyzed and the variant frequency for the indel was set above 0.25% of total reads to compare with the region of gRNA targets.

### RNA Sequencing and Data Analysis

RNA was extracted from injected mouse liver samples and prepared for RNA sequencing with TRUSEQ® Stranded mRNA sample Prep Kit (ILLUMINA®). Deep sequencing was performed on the ILLUMINA® HISEQ® platform. Single-end 50-bp reads were mapped to the UCSC mouse transcriptome (mm9) by STAR (STAR-STAR_2.4.0f1,—outSAMstrandField intronMotif—outFilterMultimapNmax 1—runThreadN 5), allowing up to 10 mismatches (which is the default by STAR). Only the reads aligned uniquely to one genomic location were retained for subsequent analysis. Expression levels of all genes were estimated by Cufflink (cufflinks v2.2.1,-p 6 -G $gtf_file—max-bundle-frags 1000000000) using only the reads with exact matches. Gene expression level was in rpkm. Mean gene expression level was obtained by averaging across replicates. The mean gene expression levels were then transformed by 1og2(rpkm+1). In R 3.3.1, Pearson's Correlation was calculated for the correlation test.

### Quantification and Statistical Analysis

### Quantification

For quantification of histological and immunohistochemistry analysis, at least three sections per tissue from 3-5 animals were analyzed using ImageJ (NIH).

### Statistical Analysis

All of the data are presented as the mean±SD or SEM and represent a minimum of three independent experiments. Statistical parameters, including statistical analysis, statistical significance, and n value, are reported in the BRIEF DESCRIPTION OF THE DRAWINGS. For in vivo experiments, n=number of animals. For ChIP-qPCR, the analytic data are means±SEM, and other data are means±SD. For statistical comparison, a two-tailed Student's t-test was performed. A value of p<0.05 was considered significant (represented as *p<0.05, **p<0.01, ***p<0.001 or not significant (n.s.)). For serum insulin levels in blood samples, statistical analyses were carried out using PRISM® 6 Software (GRAPHPAD®).

### Example 2

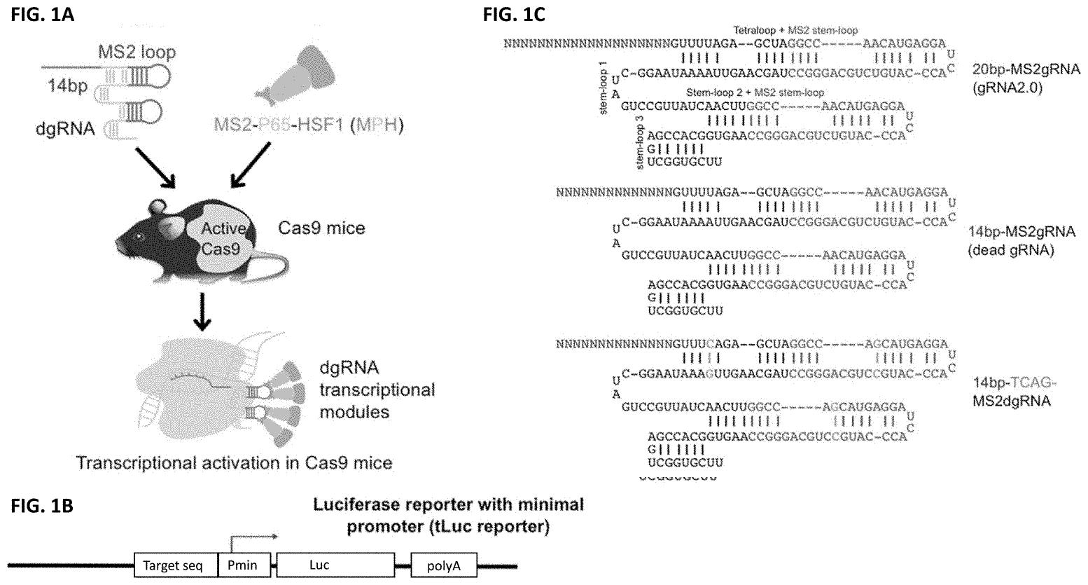

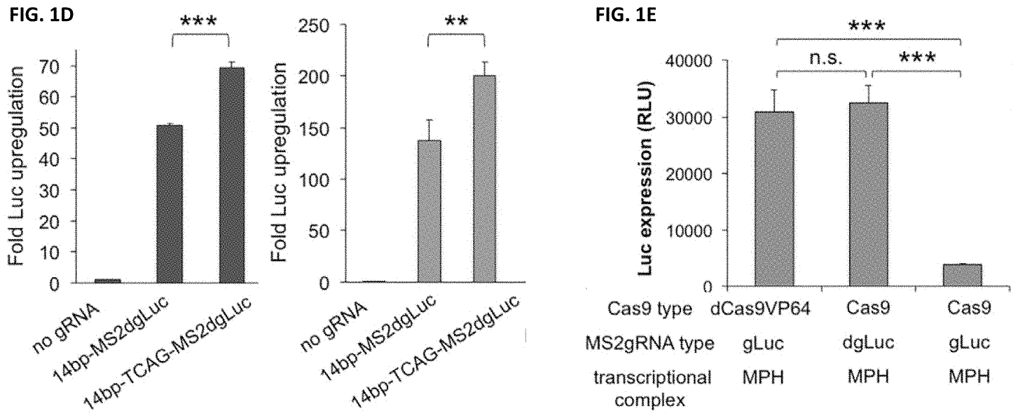

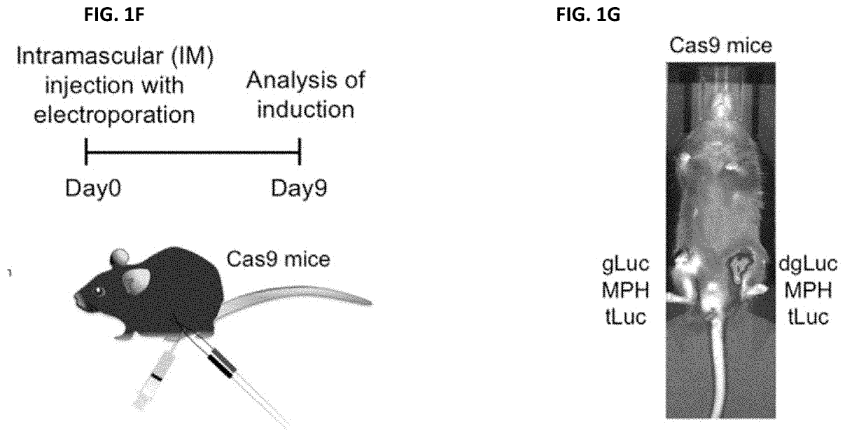

This example demonstrates a CRISPR/Cas9 system the enables target gene activation. All second-generation CRISPR/Cas9 TGA systems fuse nuclease-dead Cas9 (dCas9) to a transcriptional activation complex, which results in a coding sequence that exceeds the capacity of a single AAV. To develop a system in which the transcriptional activators were separated from dCas9, a second-generation CRISPR/Cas9-based TGA system SAM module was used (Konermann et al., 2015). The SAM system relies on an engineered hairpin aptamer that contains two MS2 domains, which can recruit the MS2:P65:HSF1 (MPH) transcriptional activation complex to the target locus. This separate, MS2-mediated transactivation complex significantly enhances the efficiency of TGA for dCas9-VP64. However, the dCas9-VP64 construct still exceeds the capacity of regular AAVs. To solve this problem, short sgRNAs (14 or 15 base pairs (bp) rather than 20 bp) were used to guide wild-type Cas9 to the target locus. These short sgRNAs prevent active Cas9 from creating a DSB (Dahlman et al., 2015; Kiani et al., 2015) and are, therefore, termed dead sgRNAs (dgRNAs). We used versions of dgRNAs engineered to contain two MS2 domains to recruit the MPH transcriptional activation complex (Dahlman et al., 2015). Herein, we refine the dgRNA system to enable high levels of in vivo TGA when introduced into mice expressing active Cas9 (FIG. 1A) (Platt et al., 2014).

First, a luciferase reporter was constructed that included a dgRNA binding site followed by a minimal promoter and a luciferase expression cassette (the tLuc reporter) (FIG. 1B). Next, MS2 dead sgRNA (MS2dgRNAs) sequences targeting tLuc were altered and screened for high levels of luciferase activity in vitro (FIGS. 1C and 8A). The MS2dgRNA scaffold was modified by changing the GC ratio, shortening repetitive sequences, or both. Based on in vitro analyses, 14bp-TCAG-MS2dgLuc provided highest level of reporter activation (FIGS. 1D and 8B). Surprisingly, activation efficiency of the dgRNA system disclosed herein was comparable to the activation efficiency of the original dCas9VP64 in combination with MPH (dCas9VP64/MPH/MS2gLuc). Thus, even without VP64, the modified Cas9/MS2dgLuc-MPH complex drove high levels of TGA (FIG. 1E). Other combinations of transcriptional activation complexes were investigated (e.g., VP64, P65, and Rta), but the MPH complex was most effective (FIGS. 8C-8F) (Chavez et al., 2015; Chavez et al., 2016; Konermann et al., 2015).

To test the efficacy of system disclosed herein in live animals, plasmids containing the luciferase reporter and plasmids containing dgLuc/MPH sequences (dgLuc will be used to represent optimized MS2dgLuc) were co-injected into hind-limb muscles of adult Cas9-expressing mice. Plasmids were electroporated into muscle cells, and luciferase activity was monitored 9 days later (FIG. 1F). The dgLuc/MPH system resulted in luciferase expression, whereas replacing dgLuc with gLuc (i.e., with a full-length target sequence) resulted in no luciferase activity (FIG. 1G).

### Example 3

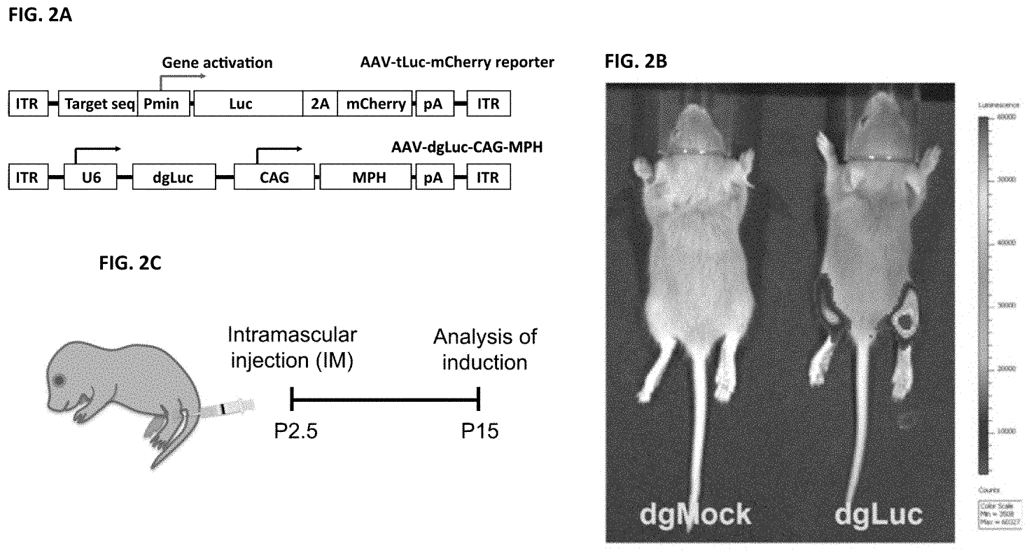

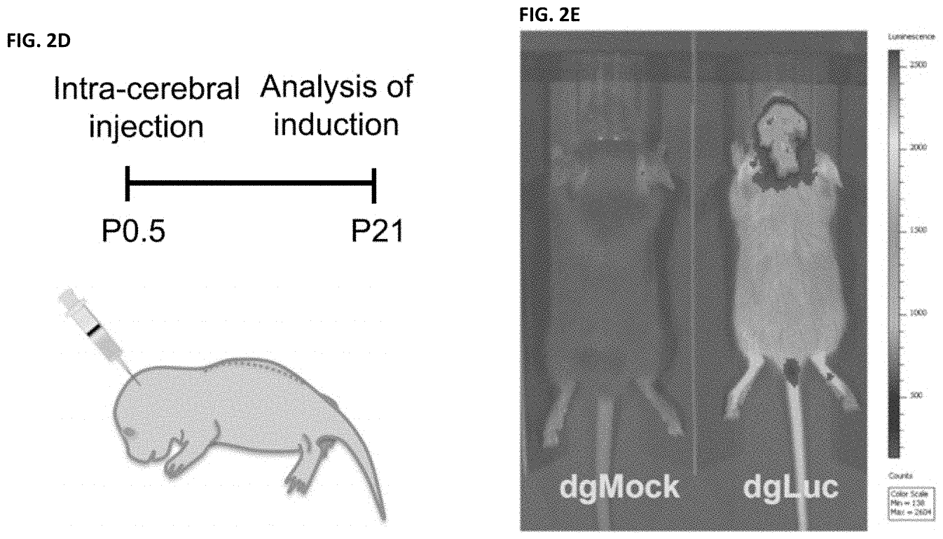

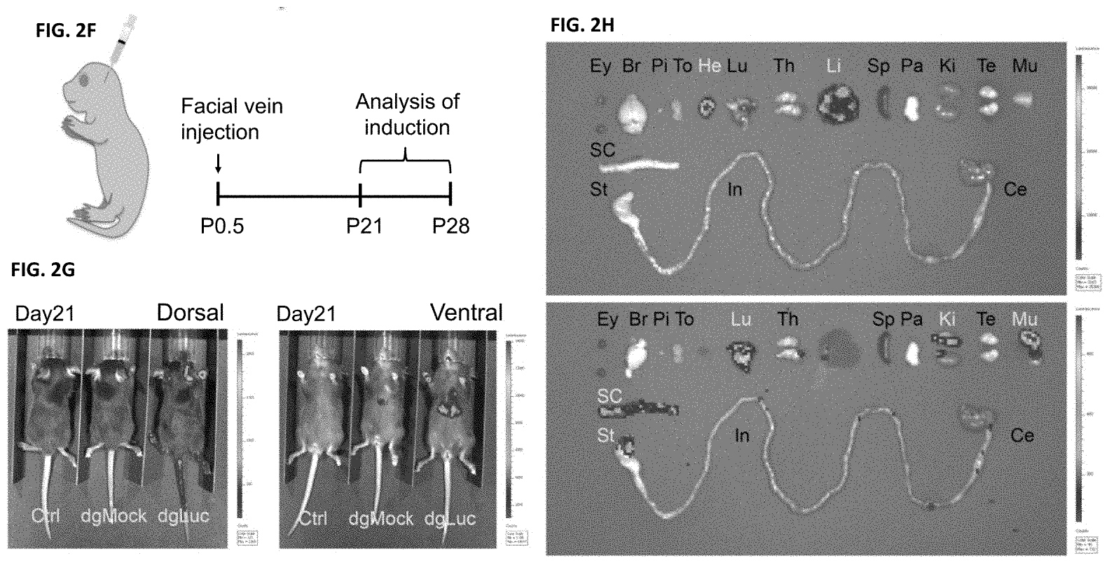

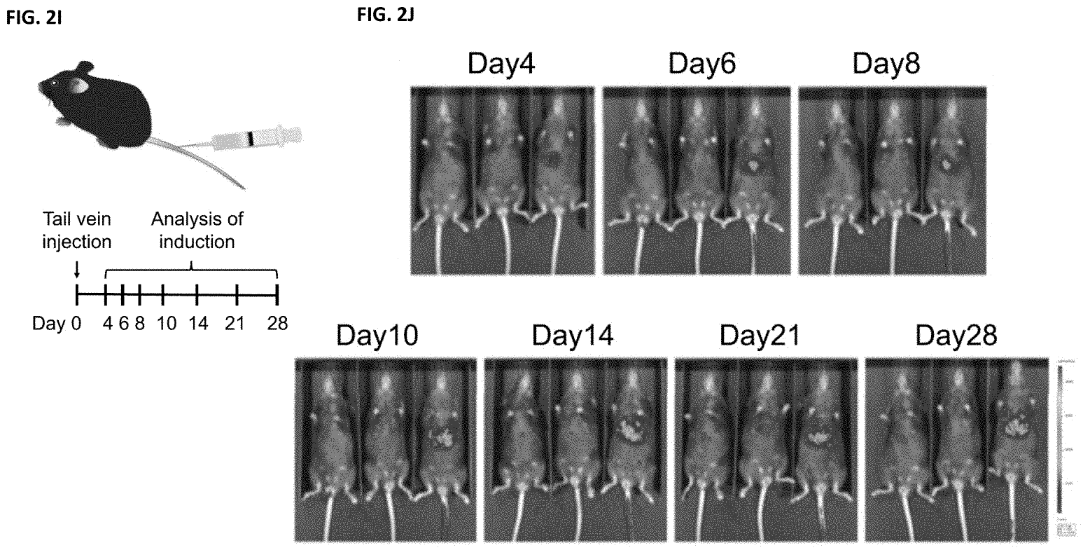

This example shows that an AAV-mediated CRISPR/Cas9 TGA system activates reporters in different organs of mice. To facilitate the in vivo delivery of the CRISPR/Cas9 TGA system and elevate expression levels, elements of the system (namely dgLuc and MPH) were introduced into an AAV, in which dgLuc and MPH expression was driven by U6 and CAG promoters, respectively (AAV-dgLuc-CAG-MPH). AAV serotype 9 was used because it infects a wide range of organs and is therapeutically safe (Zincarelli et al., 2008). To assess levels of TGA, reporter AAV was created in which luciferase and mCherry sequences were placed downstream of the dgLuc binding site (AAV-tLuc-mCherry; FIG. 2A). Next, the two AAVs were bilaterally co-injected into hind-limb muscles of Cas9-expressing neonatal mice (P2.5), and luciferase activity was assessed at P15 (FIG. 2B). Co-injection of the AAVs resulted in luciferase activity in vivo, but not when a scrambled dgRNA control (dgMock) was used (FIG. 2C). The AAVs were then directly injected into the brain of Cas9-expressing mice, and CRISPR/Cas9-mediated TGA was again detected (injections were performed at P0.5, and luciferase activity assessed at P21) (FIGS. 2D and 2E). Next, the AAVs were delivered systemically via facial vein injection into Cas9-expressing mice at P0.5. At P21, mice injected with AAV-dgLuc exhibited luciferase activity, but not those injected with AAV-dgMock (or non-injection controls) (FIGS. 2F and 2G). Organs were dissected from mice injected with AAV-dgLuc, and the highest levels of ex vivo luciferase activity were detected in the liver and heart. Lower levels were detected in the lung, kidney, muscle, spinal cord, and stomach (FIG. 2H). Finally, the AAVs were systemically delivered to adult mice via tail vein injection (FIG. 2I). Four days later, luciferase activity was detected in the liver of mice receiving AAV-dgLuc, but not AAV-dgMock (or non-injection controls) (FIG. 2J). These results demonstrate that the CRISPR/Cas9 TGA disclosed herein induces transcription of a reporter gene in vivo.

### Example 4

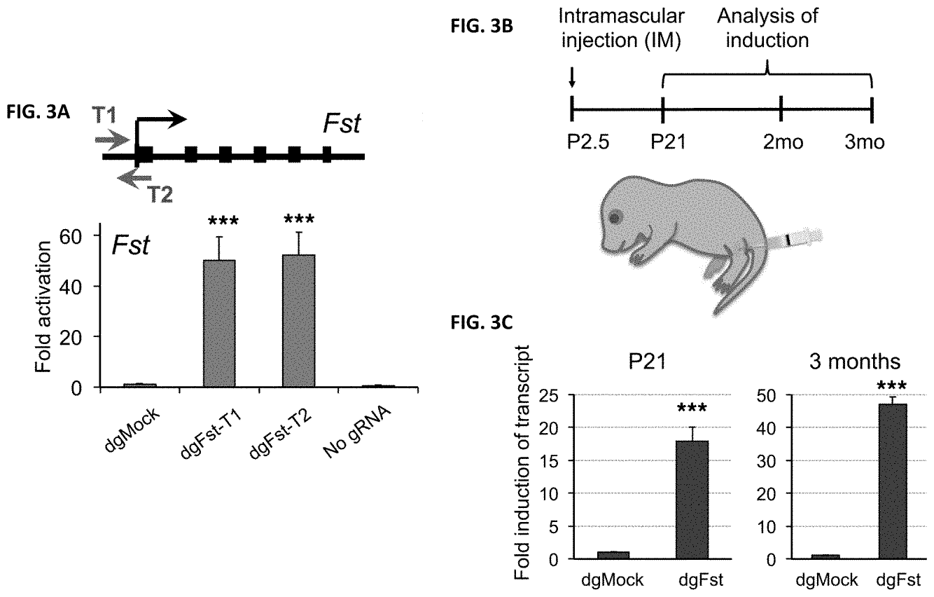

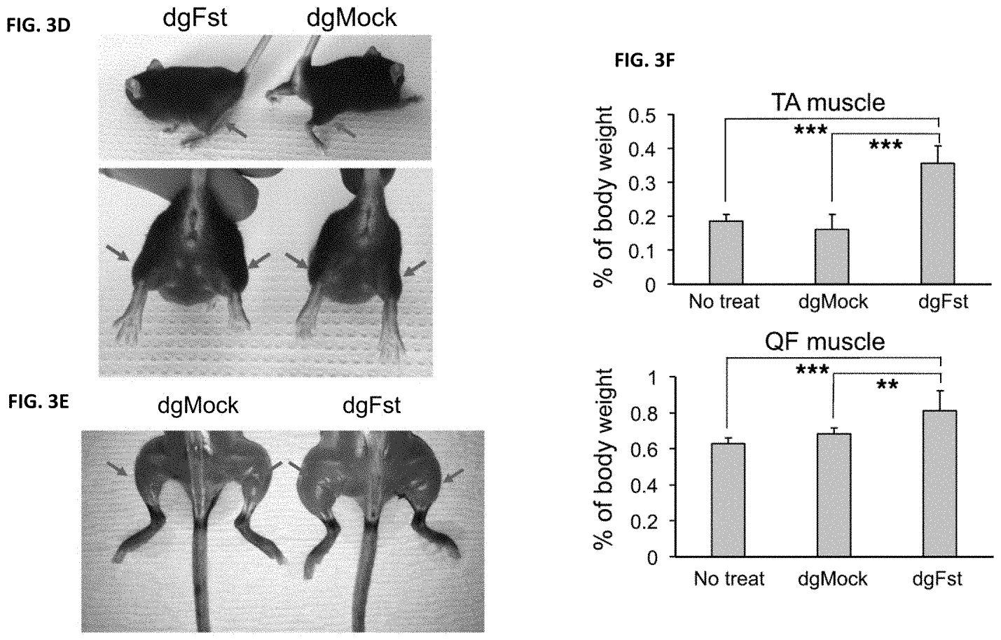

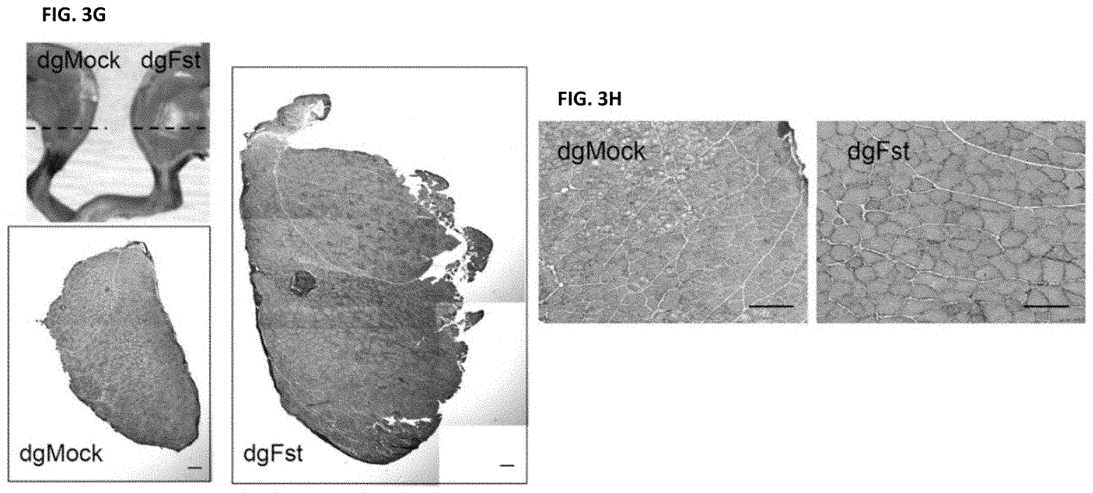

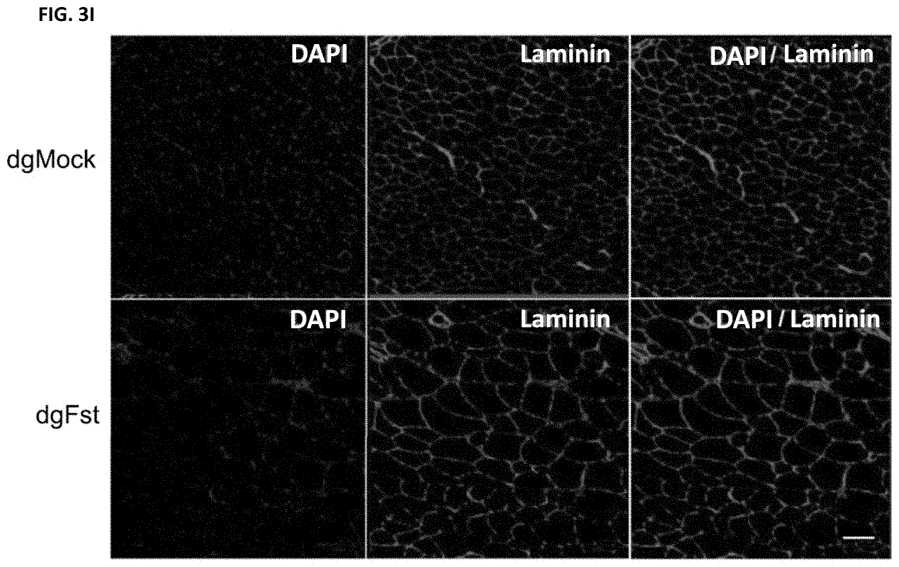

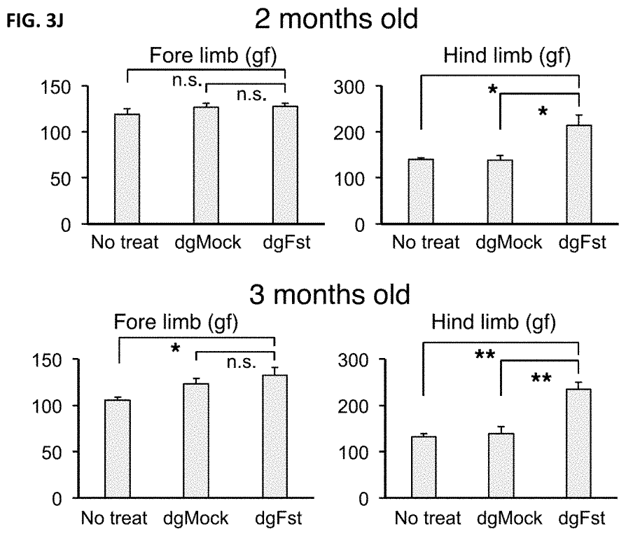

This example demonstrates phenotypic enhancement of muscle mass induced by CRISPR/Cas9 TGA in vivo. The CRISPR/Cas9 TGA system was next examined for activation of endogenous genes (rather than an exogenous reporter) and to demonstrate that induced levels of expression were sufficient to produce a phenotype. The mouse follistatin (Fst) gene was targeted because Fst overexpression increases muscle mass (Haidet et al., 2008). TGA is most effective when sgRNAs target sequences within −400 and +100 bp of the transcriptional start site (in particular between −100 and +50 bp) (Gilbert et al., 2014; Kearns et al., 2014; Konermann et al., 2015). Therefore, dgFst RNAs were constructed based on these data, and two Fst target sequences (T1 and T2) were identified near the transcriptional start site. To examine Fst activation in vitro, N2a cells stably expressing Cas9 were transfected with dgFst-T1-MPH or dgFst-T2-MPH plasmids. The controls included dgMock-MPH and a no transfection group. Comparable levels of Fst expression (approximately 50-fold up-regulation compared with the controls) were observed with the two dgFst-MPH constructs (FIG. 3A). AAV-dgFst-T2-MPH was then delivered via intramuscular (IM) injection into the hind-limb of Cas9-expressing mice at P2.5 (FIG. 3B). At P21, increased levels of Fst expression (approximately 18-fold compared with gMock controls) were observed in hind-limbs injected with AAV-dgFst-T2-MPH (FIG. 3C). At P45, a gross increase in muscle mass was observed (FIG. 3D). Increases in Fst expression and muscle mass were also observed 3 months following the injections (FIGS. 3C, 3E, and 3F). AAV-dgFst-T2-MPH injection increased muscle fiber size in the tibialis anterior (TA) muscle (FIGS. 3G-3I) and increased hind-limb muscle strength compared with the controls. Less or no difference in the strength of fore limbs, which were not injected, was observed in these animals (FIG. 3J).

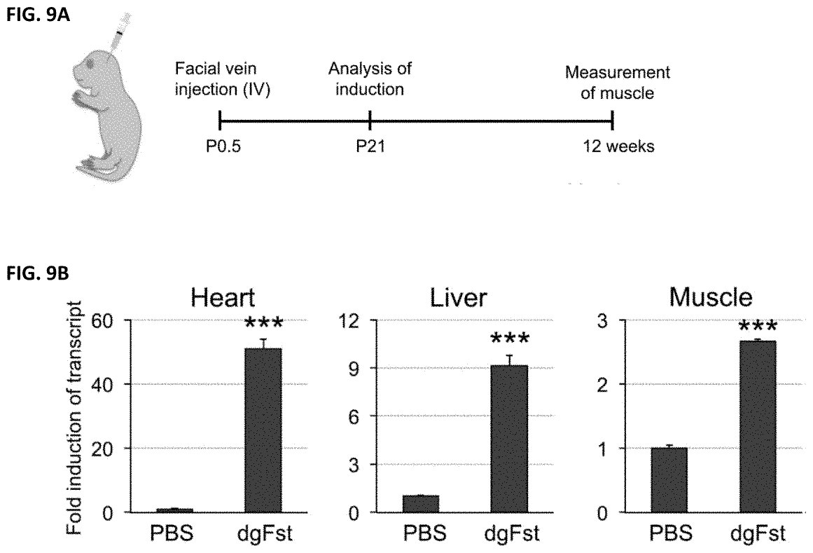

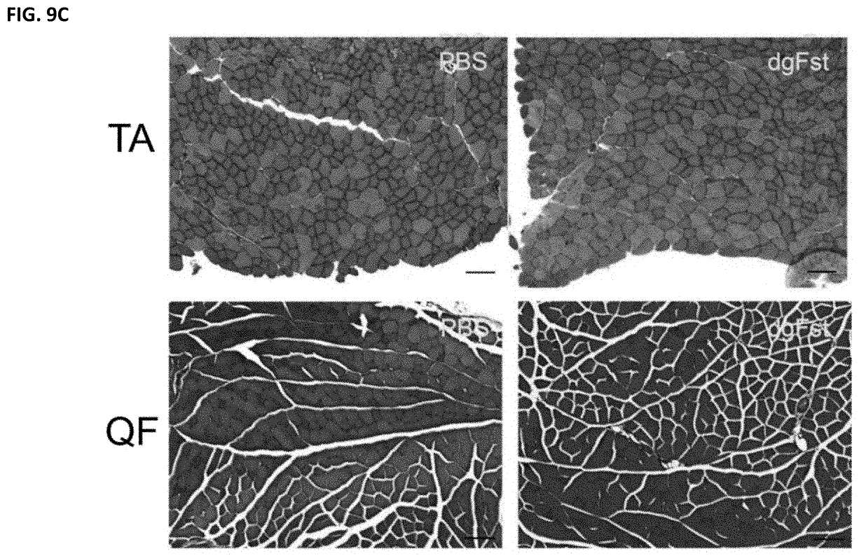

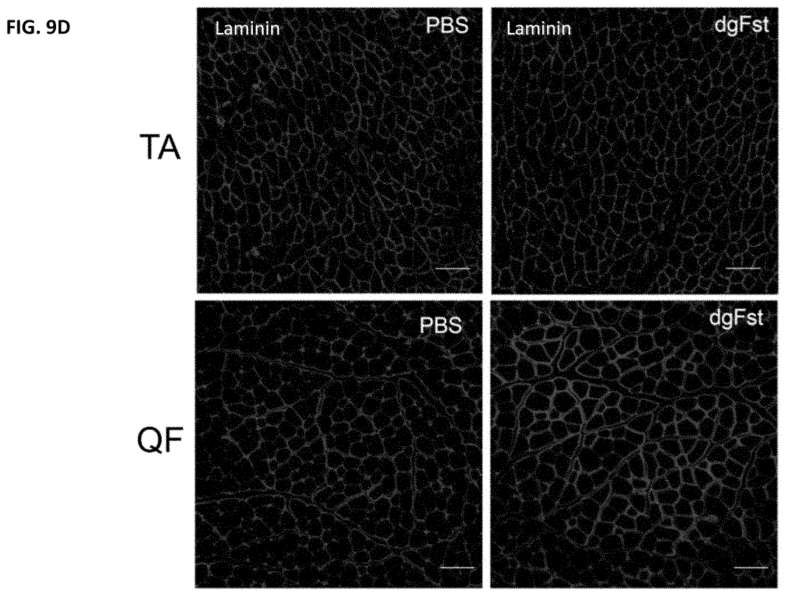

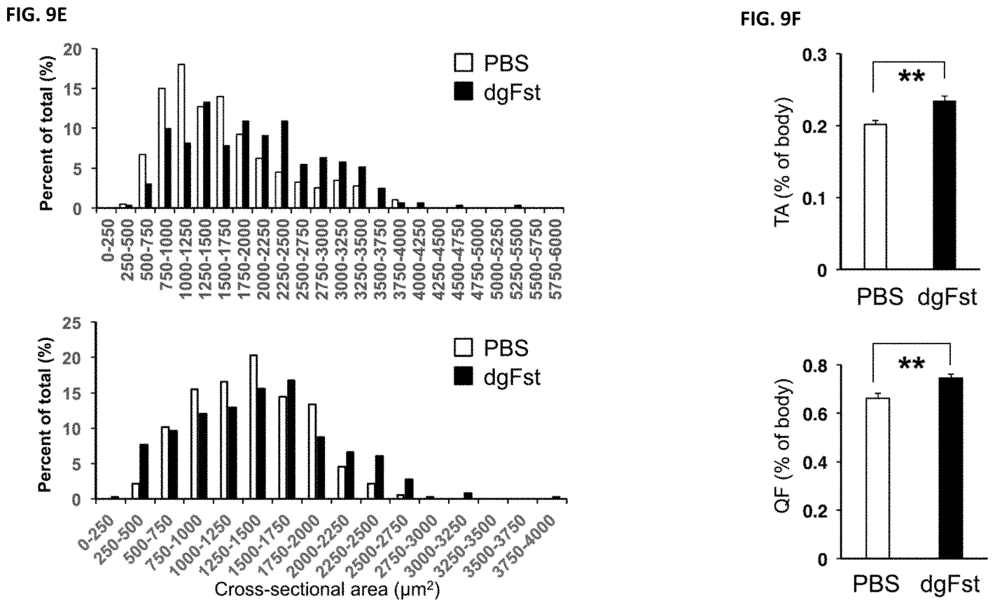

To demonstrate the effect on Fst expression when the AAV was delivered systemically, AAV-dgFst-T2-MPH was administered to Cas9-expressing mice at P0.5 via facial vein injection. At P21, heart, liver, and muscle tissues were dissected, and Fst expression was analyzed (FIG. 9A). Fst expression levels were elevated 45-fold, 9-fold, and 2.7-fold in heart, liver, and muscle tissues, respectively (compared with control PBS-injected mice) (FIG. 9B). Twelve weeks after the injection, increases in muscle fiber size were observed in the TA and quadriceps femoris (QF) muscles, compared with PBS controls (FIGS. 9C-9E). Finally, 12 weeks after the injection, Fst overexpression led to increases in the relative weights of the TA and QF muscles (FIG. 9F).

### Example 5

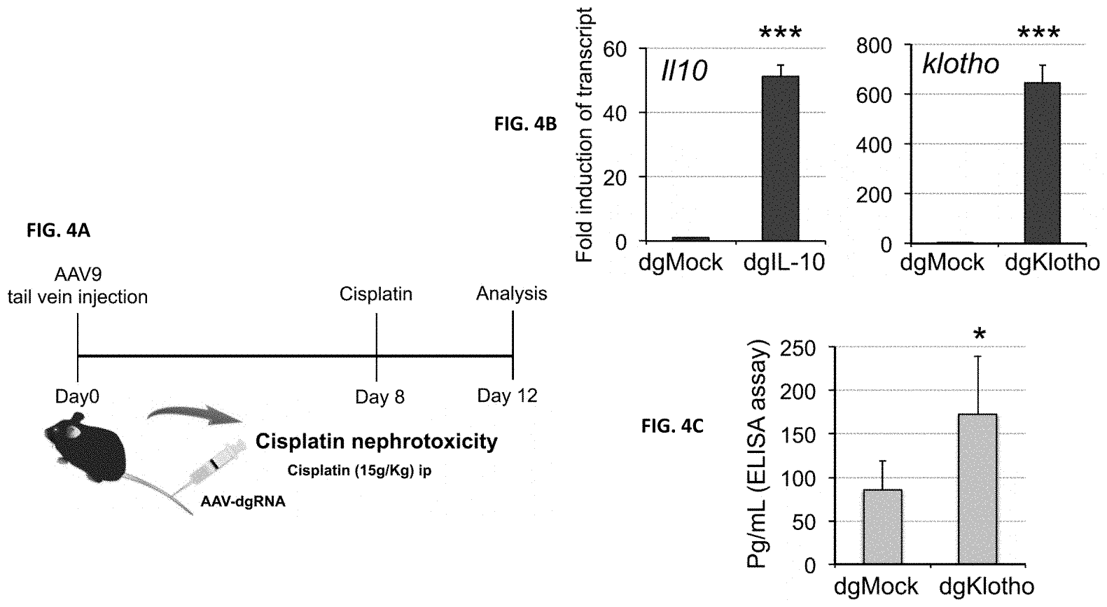

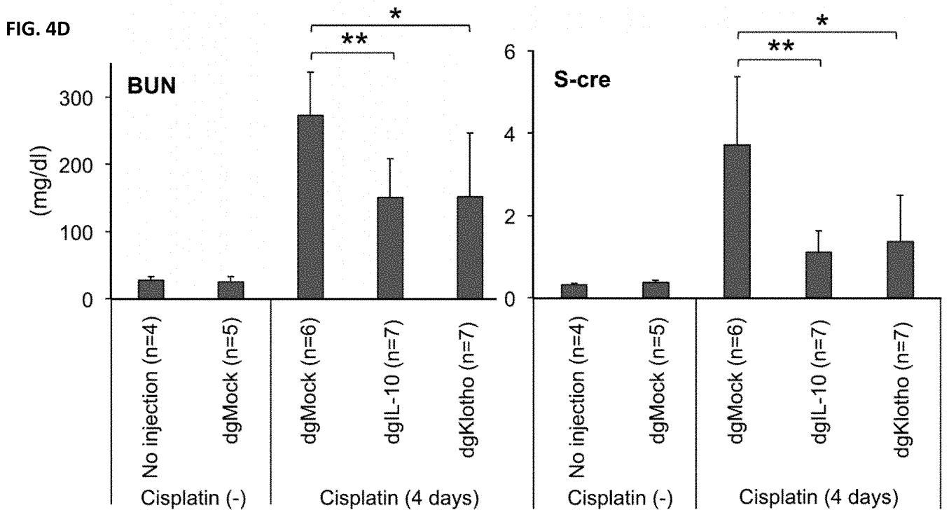

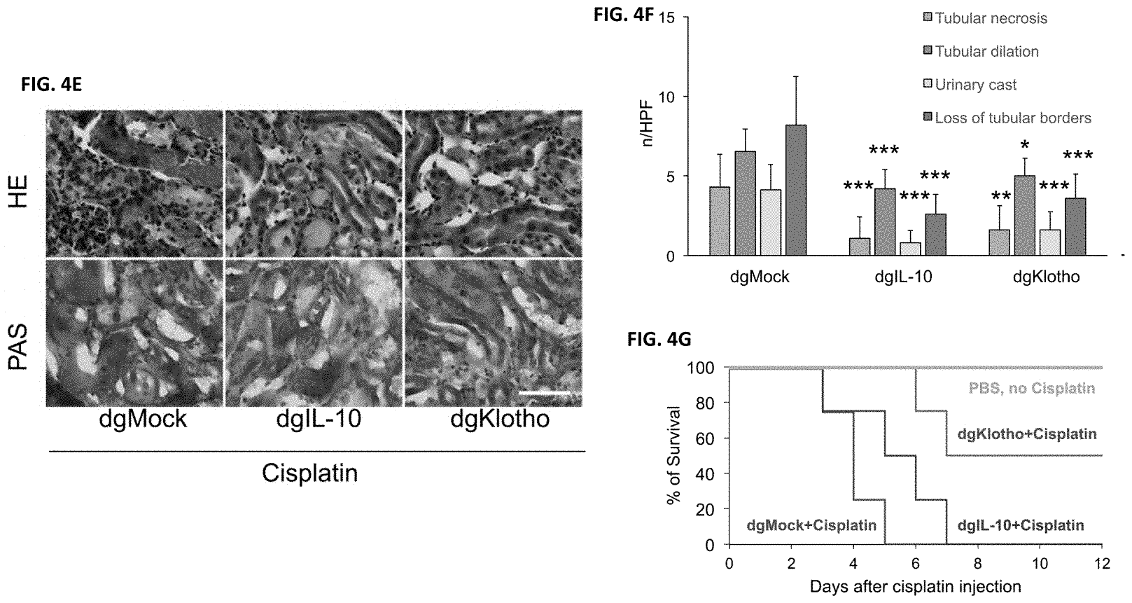

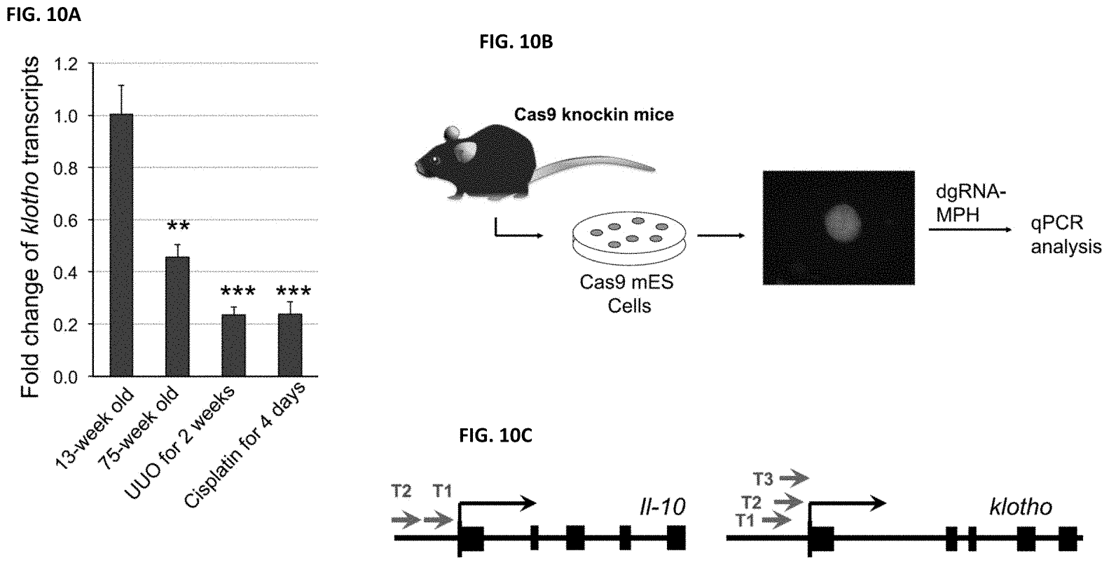

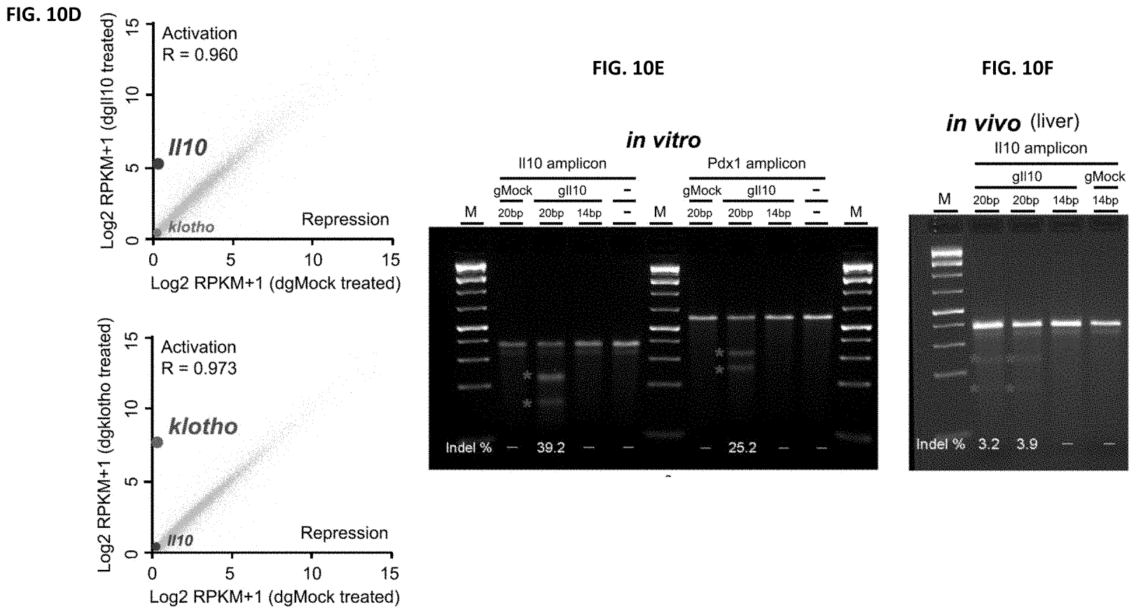

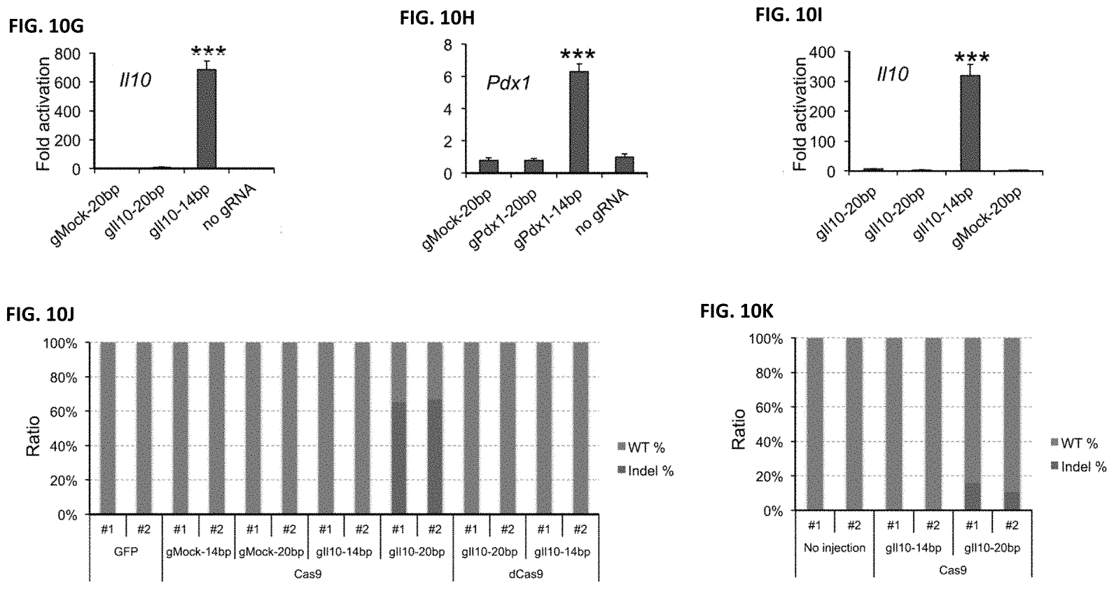

This example shows that induction of IL-10 or Klotho expression via CRISPR/Cas9 TGA in vivo can ameliorate acute kidney injury. To demonstrate therapeutic applications of CRISPR/Cas9 TGA, mouse models were used to show amelioration of human diseases. First, a mouse model of acute kidney injury was used, targeting the genes klotho and interleukin 10 (Il10). Klotho protects against renal damage, and expression of this gene is reduced following ageing and acute kidney injury (FIG. 10A) (Kurosu et al., 2005; Panesso et al., 2014). IL-10 is an anti-inflammatory cytokine that ameliorates renal injury following cisplatin treatment (Jin et al., 2013; Ling et al., 2011). The CRISPR/Cas9 TGA system was used to investigate induction of Klotho or IL-10 expression in vivo to treat acute kidney injury. For these experiments, mouse embryonic stem cell lines were first derived from Cas9-expressing mice (Cas9 mESCs) and used to examine gene induction by dgRNAs targeting klotho or Il10 (FIG. 10B and 10C). The most effective klotho and Il10 dgRNAs from these in vitro assays were then assembled into AAV vectors (AAV-dgKlotho-MPH and AAV-dgIL-10-MPH), and viruses were injected into the tail vein of adult Cas9 mice (FIG. 4A). The specificity of in vivo TGA was first assessed using RNA-seq. Twelve days following the injection, liver tissue was dissected. Compared with AAV-dgMock controls, target genes were dramatically upregulated (158-fold for Il10 and 2553-fold for klotho on average), indicating high levels of TGA in vivo (FIG. 10D). In addition, mice injected with AAV-dgIL-10-MPH exhibited no induction of klotho (and vice versa), indicating no crosstalk between these reagents. Next, to demonstrate that truncated dgRNAs do not cause DSBs in the presence of active Cas9, either sgRNAs or MS2-dgRNAs together were transduced and analyzed using the SURVEYOR® assay and deep sequencing. The results showed that sgRNAs disrupted target genes (via DSB-induced indel generation), whereas MS2-dgRNAs induced TGA with undetectable levels of DSBs (in vitro and in vivo samples) (FIGS. 10E-10K).

To assess the therapeutic benefit, acute kidney injury was induced in mice via cisplatin injection 8 days after AAV injection (FIG. 4A). AAV injection elevated levels of klotho and Il10 gene expression in the liver (FIG. 4B) and elevated levels of Klotho protein secreted into the serum (FIG. 4C). Overexpression of Klotho or IL-10 in cisplatin-treated mice resulted in improved renal function, as blood urea nitrogen (BUN) and serum creatinine (S-Cre) levels were significantly lower compared with dgMock controls (FIG. 4D). Moreover, kidneys from these mice were dissected and histologically analyzed. Overexpressing Klotho or IL-10 improved a number of pathological features (namely tubular necrosis, tubular dilation, urinary cast, and loss of tubular borders) compared with controls (FIGS. 4E and 4F). Importantly, AAV treatment extended the mouse survival time following a high dose of cisplatin treatment; this effect was most dramatic with Klotho (FIG. 4G). Thus, the CRISPR/Cas9 TGA system induced the expression of functional proteins in vivo, and levels of Klotho/IL-10 overexpression were sufficient to provide prophylactic interventions of disease pathogenesis in a mouse model of acute kidney injury.

### Example 6

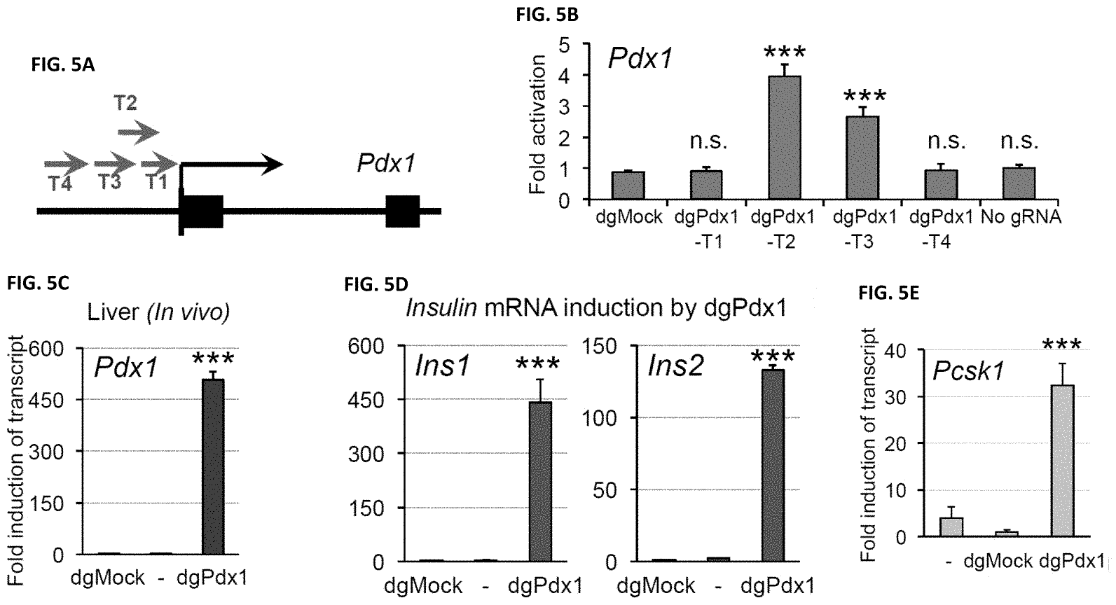

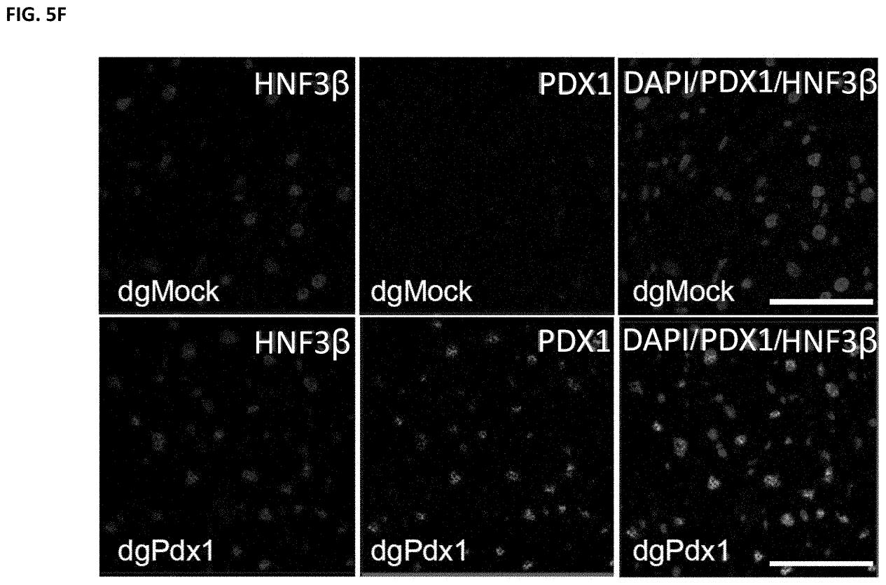

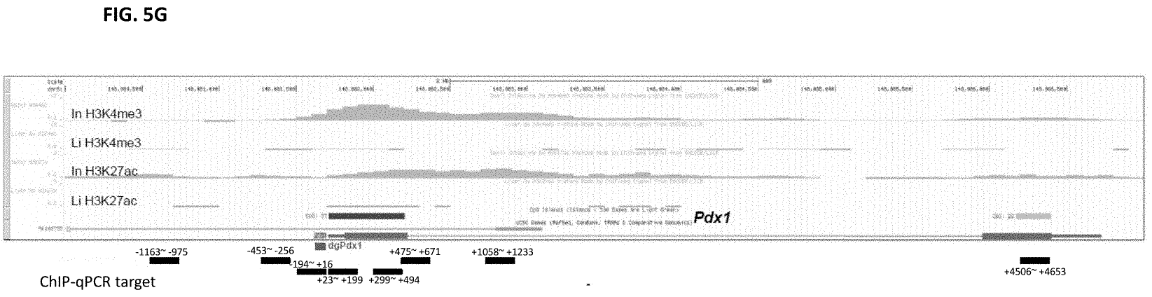

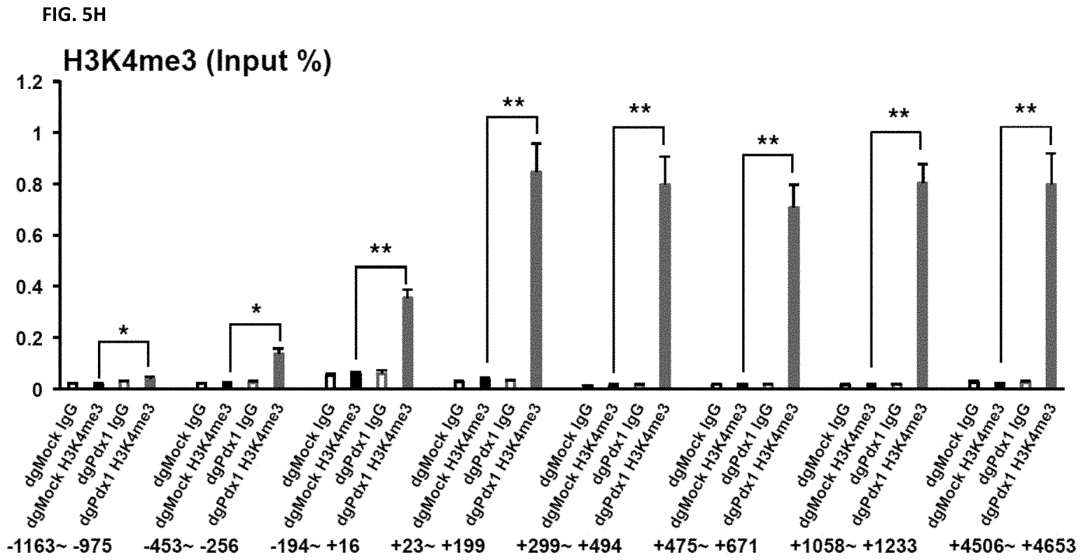

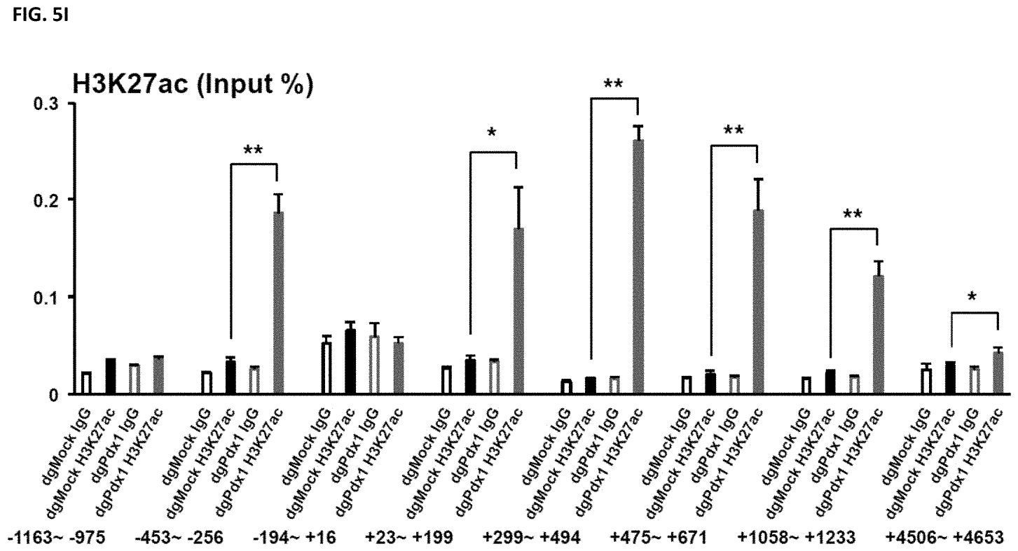

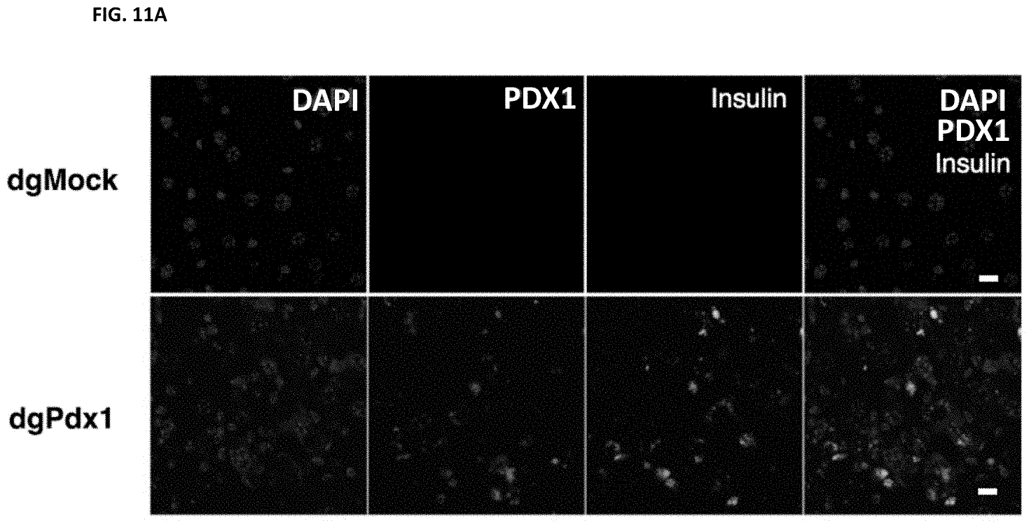

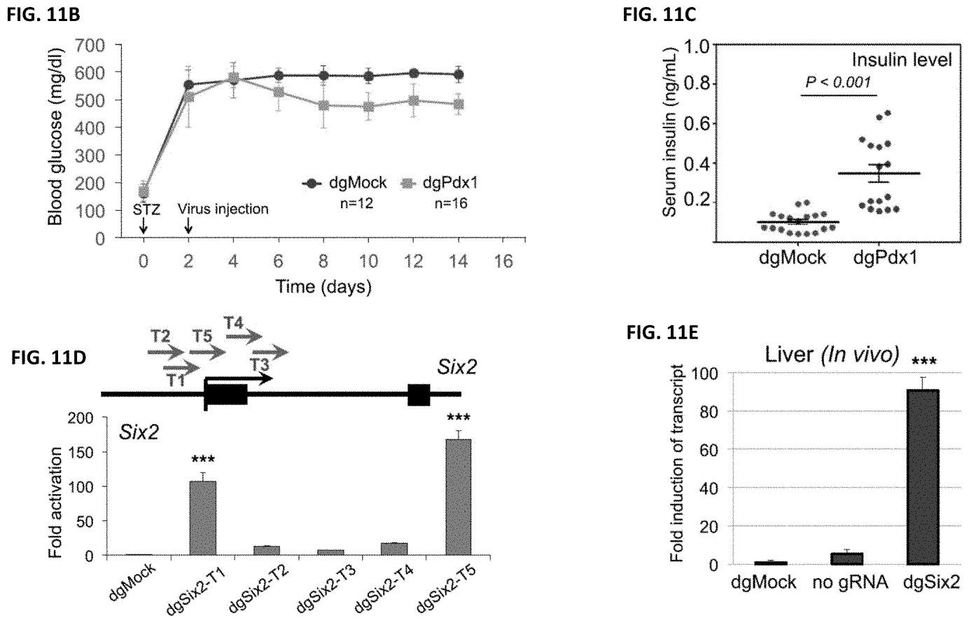

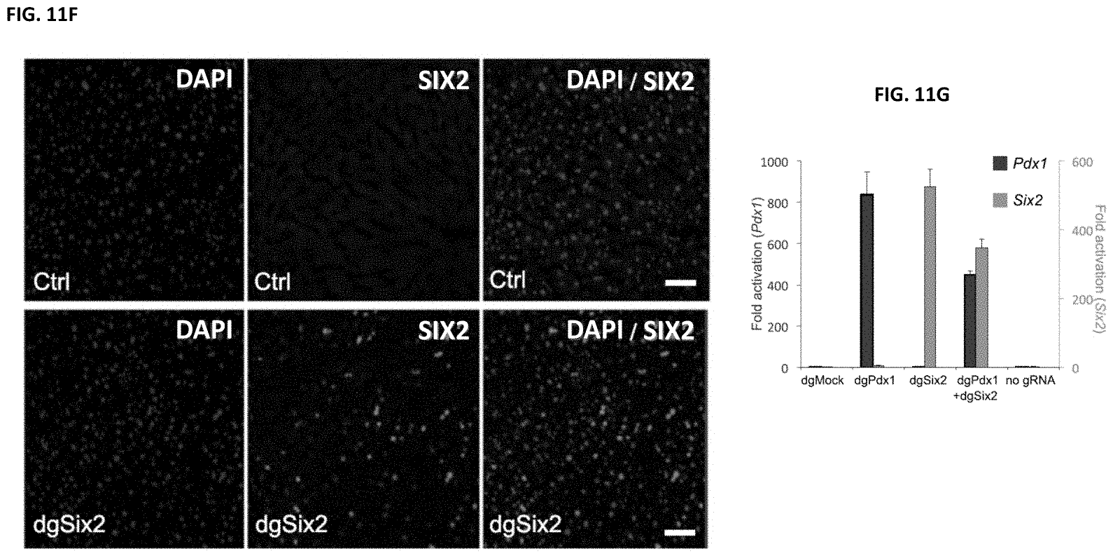

This example demonstrates that CRISPR/Cas9 TGA results in transdifferentiation of liver cells into insulin-producing cells in vivo via trans-epigenetic modulation. Next, activation of an endogenous gene via CRISPR/Cas9-mediated TGA was shown to produce in vivo transdifferentiation of cells. Pancreatic and duodenal homeobox gene 1 (Pdx1) was overexpressed in liver cells (using AAV-dgPdx1-MPH) to generate insulin-secreting cells to treat a mouse model of type I diabetes. Pdx1 is necessary for pancreatic development and can transdifferentiate hepatocytes into pancreatic beta-like insulin-producing cells (Ferber et al., 2000; Tang et al., 2006). First, effective dgRNAs against Pdx1 were first identified using Cas9 mESCs in vitro. Injecting AAV-dgPdx1-MPH into adult Cas9-expressing mice via tail vein injection elevated levels of Pdx1 in liver cells compared with dgMock controls (FIGS. 5A-5C and 5F). In addition to overexpressing Pdx1, AAV-dgPdx1-MPH resulted in the upregulation of insulin 1 (Ins1), insulin 2 (Ins2), and proprotein convertase subtilisin/kexin type 1 (Pcsk1) in liver cells; the latter participates in insulin processing (FIGS. 5D, 5E and 11A).

To demonstrate that the in vivo TGA system also affected epigenetic marks near the targeted genomic locus, chromatin-immunoprecipitation (ChIP)-qRT-PCR of liver samples from mice injected with AAV-dgPdx1-MPH was performed. H3K4me3 and H3K27ac epigenetic marks, which are typically associated with transcriptionally active genes, were enriched at the Pdx1 locus of AAV-dgPdx1-MPH injected mice compared with AAV-dgMock controls (FIGS. 5G-5I). These patterns of epigenetic modifications are similar to Pdx1-expressing tissues (e.g., small intestine) (FIG. 5G). Thus, the CRISPR/Cas9 TGA system transcriptionally activated a gene that is normally silent in a target organ via trans-epigenetic remodeling histone marks.

When mice were administered AAV-dgPdx-1-MPH two days following streptozotocin (STZ) treatment (160 mg/kg), which induces hyperglycemia and creates a mouse model of type I diabetes, the treated mice exhibited lower blood glucose levels than dgMock controls; thus, the mice with STZ-induced hyperglycemia were partially rescued (FIG. 11B). In addition, serum insulin levels were higher in STZ-treated mice that received AAV-dgPdx1-MPH (FIG. 11C), indicating that the AAV treatment transformed liver cells into insulin-secreting cells. The CRISPR/Cas9 TGA system can, therefore, provide a means of in vivo cell fate engineering to produce cell types necessary to restore particular physiological functions.

To further demonstrate the utility of the in vivo CRISPR/Cas9 TGA system, more than one gene was simultaneously overexpressed. dgRNAs were designed to overexpress Six2 (AAV-dgSix2-MPH), a transcription factor expressed in the kidney (FIGS. 11D-11F) (Kobayashi et al., 2008). AAV-dgPdx1-MPH and AAV-dgSix2-MPH were co-injected into the tail vein of Cas9-expressing mice. Both genes were overexpressed in the liver, demonstrating multiplexed in vivo TGA (FIG. 11G). Together, these results indicate that the CRISPR/Cas9 TGA system can be used to activate multiple endogenous genes in vivo and that TGA as well as targeted gene knockout can be achieved simultaneously in Cas9-expressing mice.

### Example 7

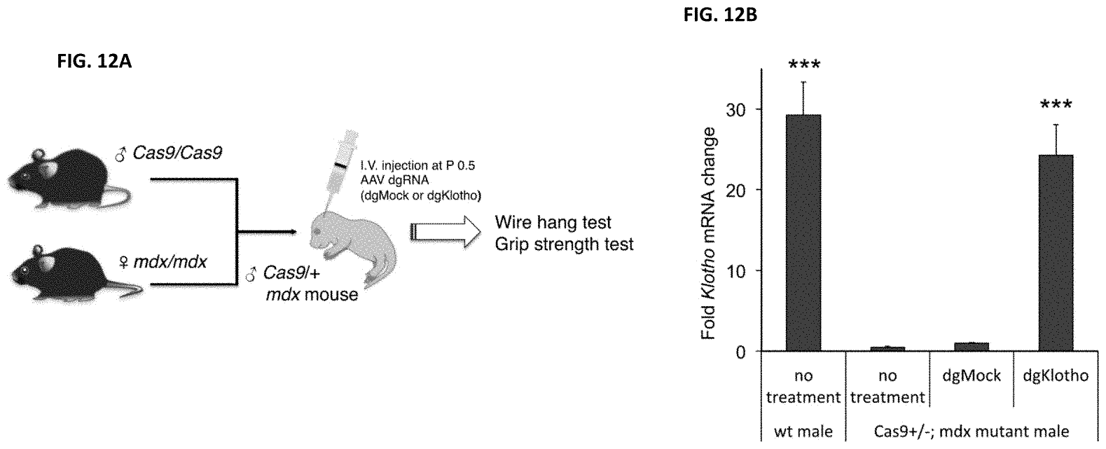

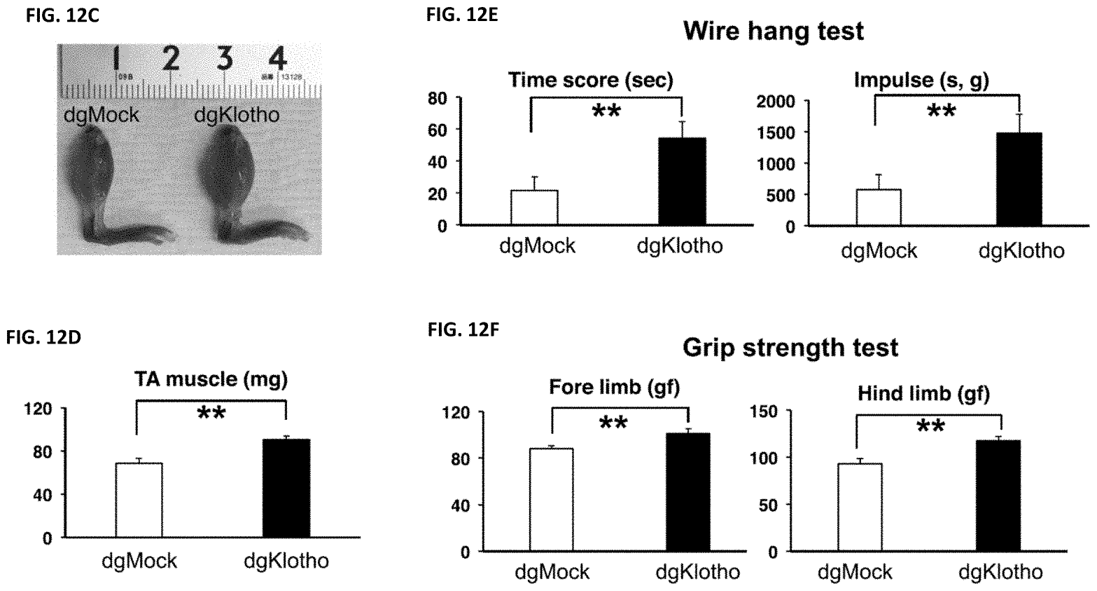

This example shows that CRISPR/Cas9 TGA of Klotho and utrophin partially rescues dystrophin-deficient mice. Next, the system disclosed herein was assayed for ameliorating disease phenotypes in mouse models of human genetic disorders. The mdx mouse model of Duchenne muscular dystrophy (DMD) (Sicinski et al., 1989) was used. DMD is a lethal, inherited muscle wasting disorder resulting from a loss-of-function mutation in the large gene, dystrophin (the cDNA is ˜14 kb). Due to the large size of this gene, in previous research, delivering a fully functional dystrophin transgene via traditional virus-mediated gene therapies was challenging (Janghra et al., 2016; Sicinski et al., 1989). Previous research has produced no effective therapy for DMD and shows the difficulty with transplanting muscle stem cells into damaged organs to stop disease progression (Sienkiewicz et al., 2015). Recent studies demonstrate that klotho is epigenetically silenced in muscle cells of mdx mice at the time of disease onset, and systemic expression of klotho via a transgene can relieve disease symptoms (Wehling-Henricks et al., 2016). Therefore, AAV-dgKlotho-MPH was injected into neonatal Cas9/mdx mutant mice via facial vein injection. This AAV restored klotho expression in muscle tissue (FIGS. 12A and 12B), increasing TA muscle mass compared with dgMock controls (FIGS. 12C and 12D). Two months after Cas9/mdx mice received AAV-dgKlotho-MPH, they showed improved muscle strength based on wire-hang and grip strength tests, compared with dgMock controls (FIGS. 12E and 12F). CRISPR/Cas9-mediated activation of the endogenous klotho gene, therefore, ameliorated DMD phenotypes, partially rescuing this mouse model of a human genetic disorder.

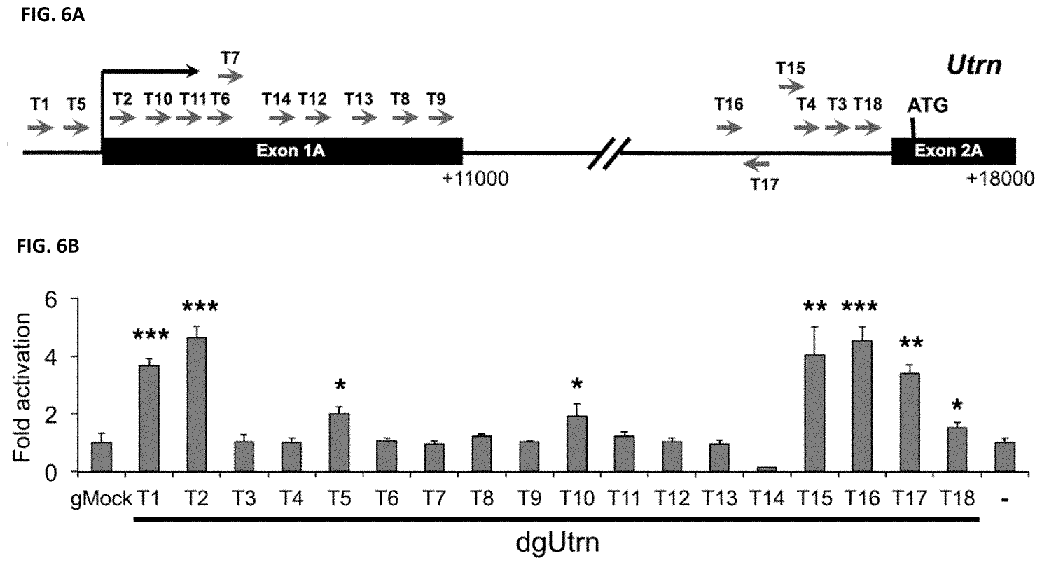

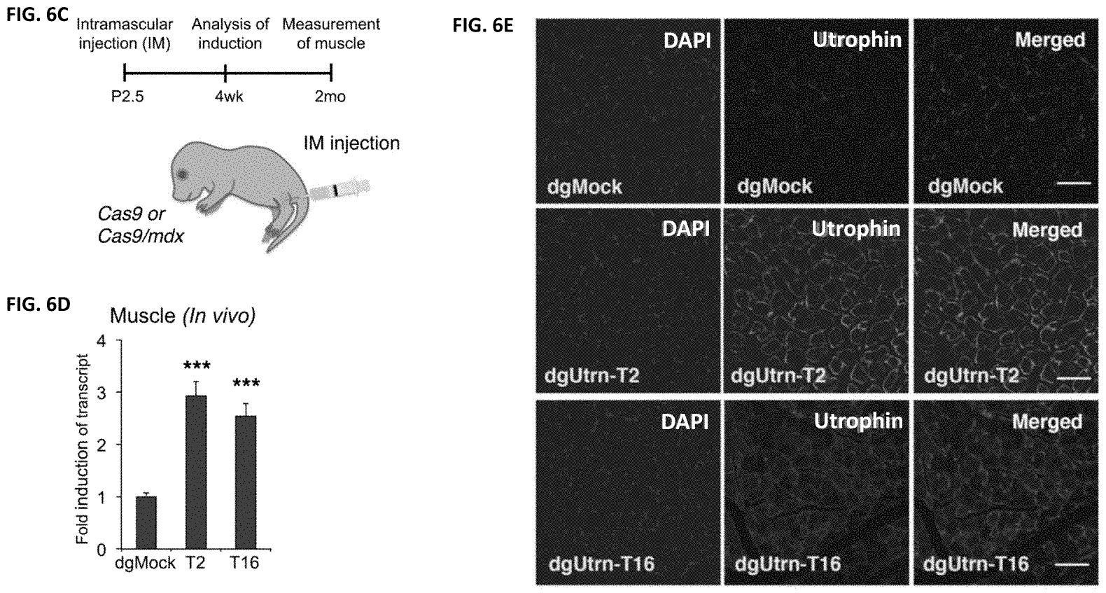

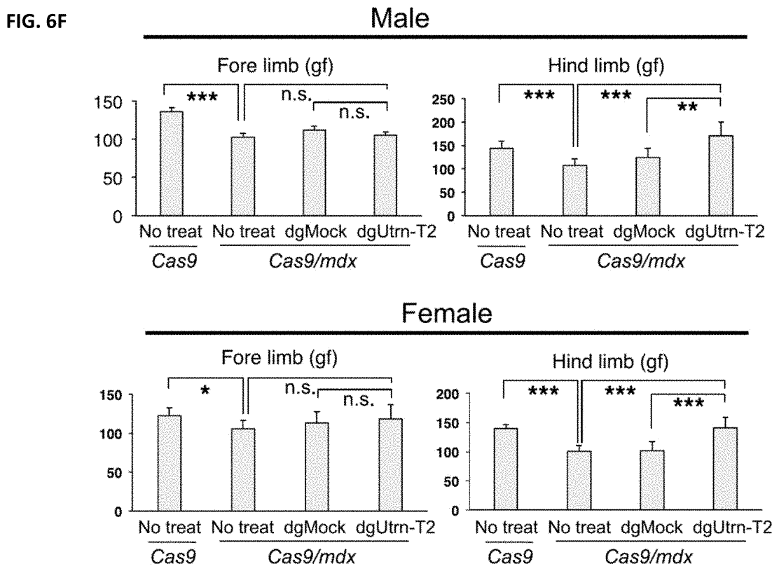

An alternative way of treating DMD is to upregulate utrophin, as the utrophin and dystrophin genes encode similar proteins (˜80% similarity), and systemic expression of utrophin in a transgenic model relieves disease symptoms (Rafael et al., 1998; Tinsley et al., 1996). As with dystrophin, the utrophin cDNA is too large to package into most viral vectors for traditional gene therapy. To overcome this hurdle, the in vivo CRISPR/Cas9 TGA described herein was used to activate the endogenous utrophin gene to compensate for the loss of dystrophin. First, 18 dgRNAs were created to identify the most effective utrophin target sites (FIG. 6A). Among these utrophin dgRNAs, T2 and T16 were most promising (FIG. 6B) (Burton et al., 1999). AAV-dgUtrn-T2-MPH or AAV-dgUtrn-T16-MPH was administered via IM injection into Cas9-expressing mice; both induced utrophin expression in muscle compared with dgMock controls (FIGS. 6C-6E). Next, AAV-dgUtrn-T2-MPH was injected into the hind limbs of Cas9/mdx mice (at P2.5). Two months later, the treated mice exhibited improved hind-limb grip strength compared with Cas9/mdx controls (untreated or AAV-dgMock-MPH). No effect on grip strength was observed for non-injected fore limbs (FIG. 6F).

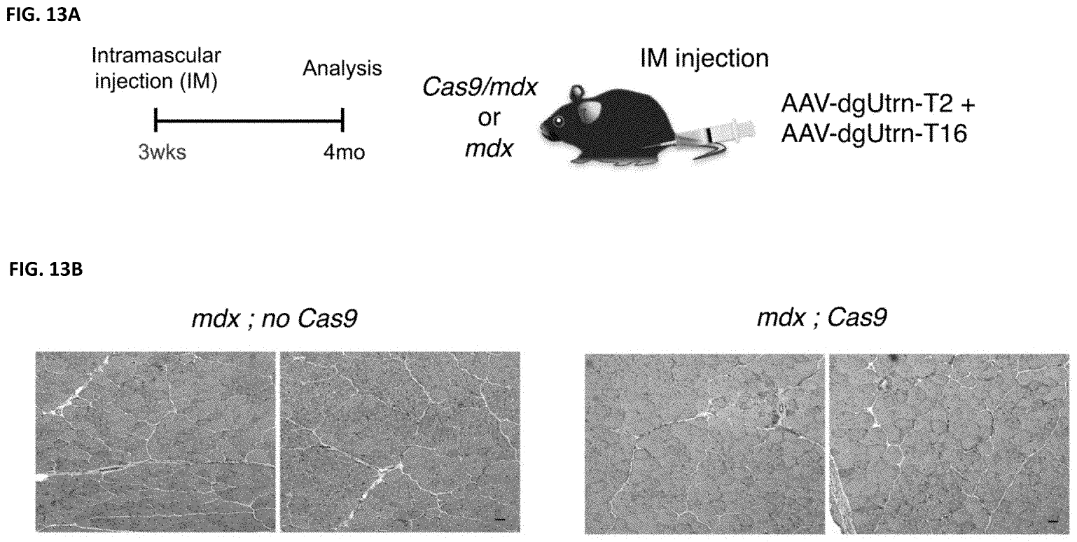

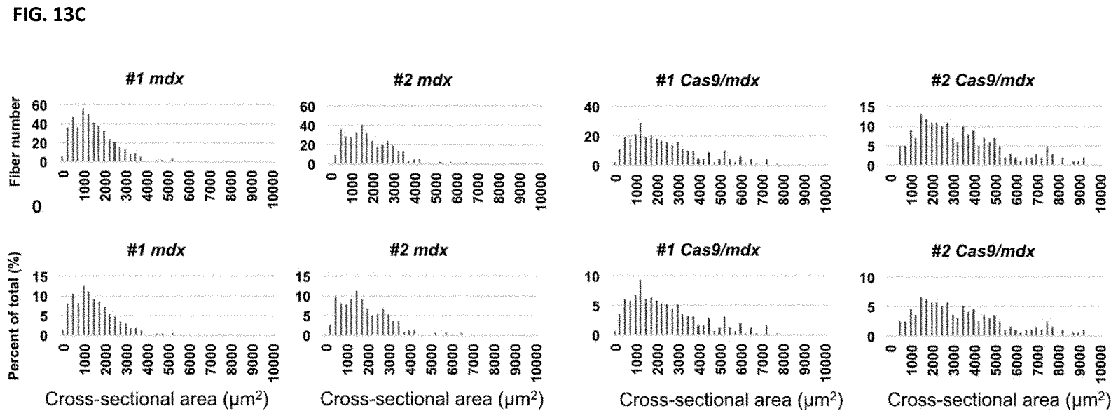

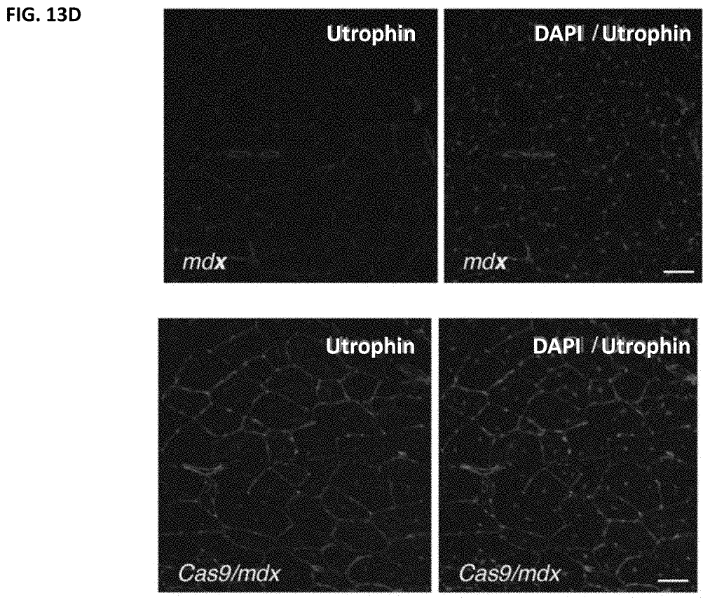

TGA-mediated up-regulation of utrophin was then assayed for rescue of mdx mice after the pathophysiology was established. AAV-dgUtrn-T2 and AAV-dgUtrn-T16 were injected together into the hind limbs of 3-week-old Cas9/mdx and mdx littermates. Disease symptoms were reduced for Cas9/mdx mice, but not for mdx controls, which lacked Cas9 (FIGS. 13A-13D).

### Example 8

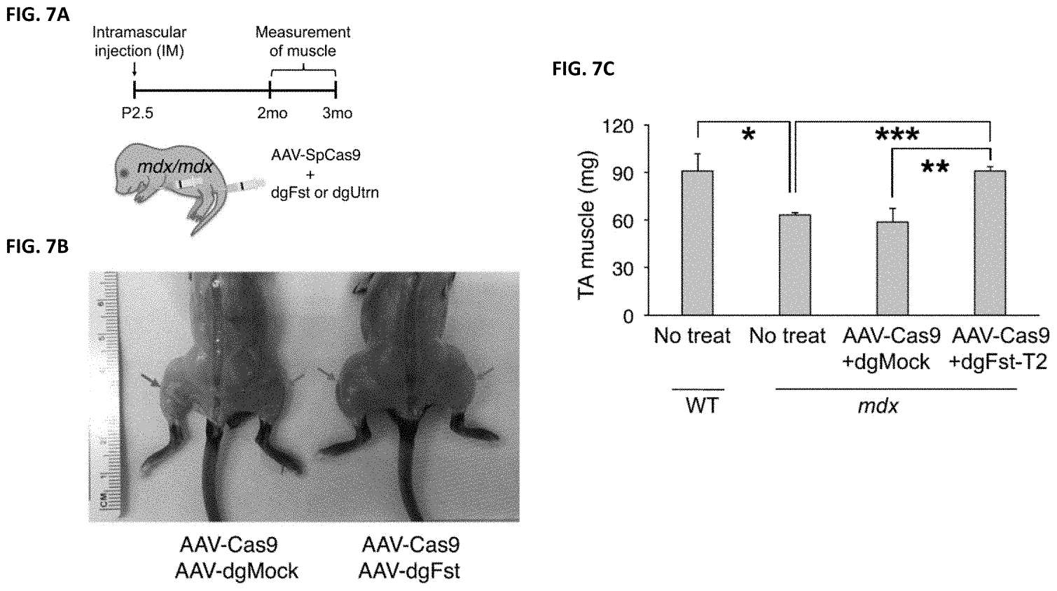

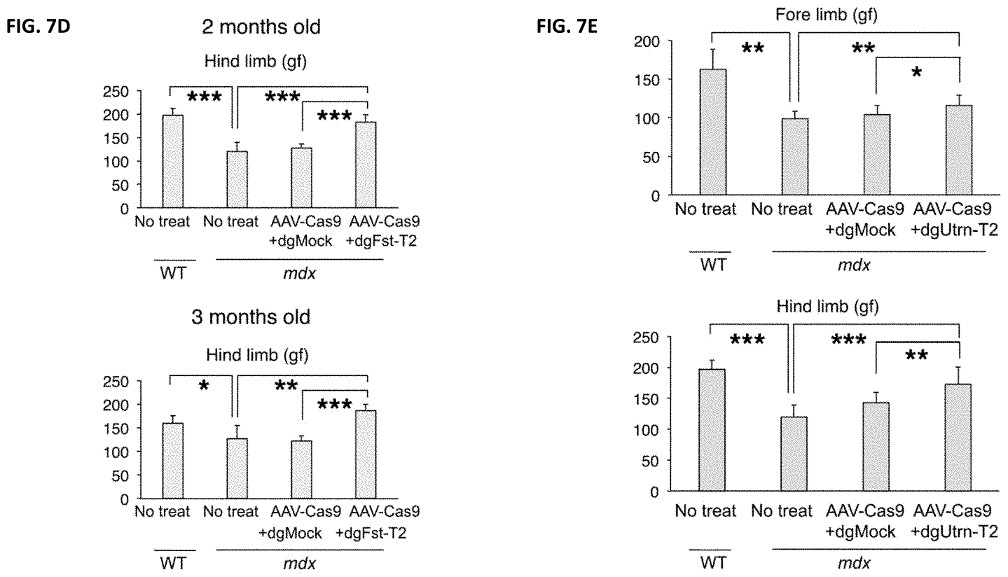

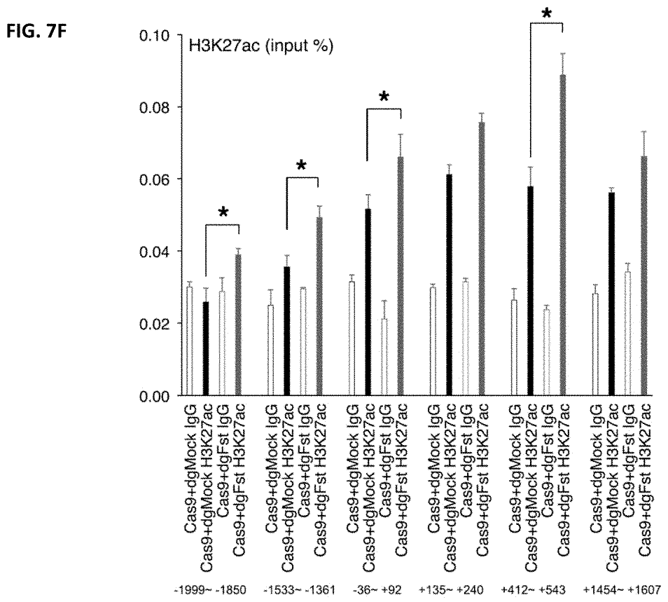

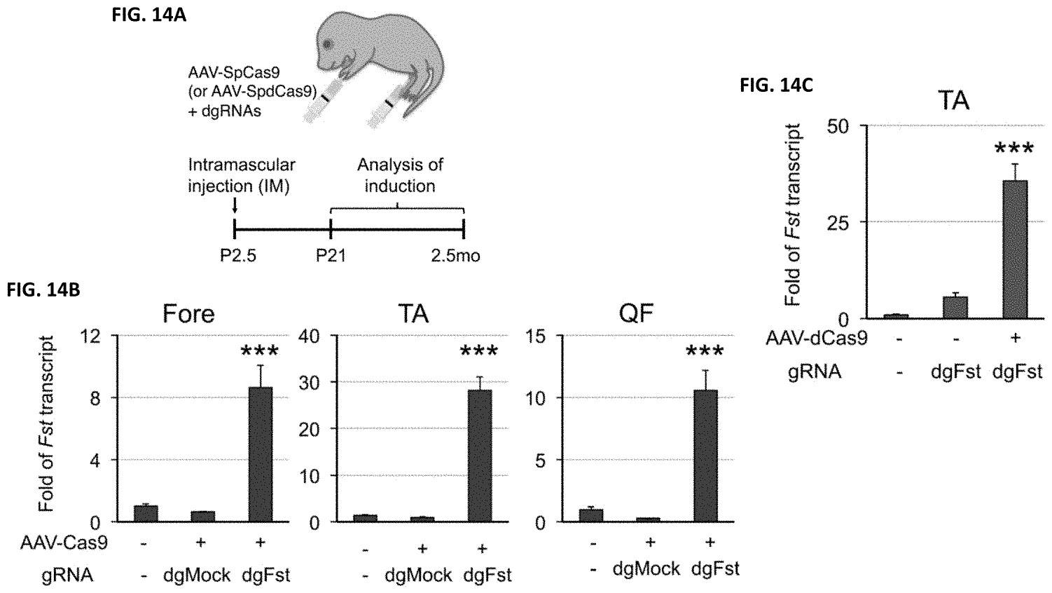

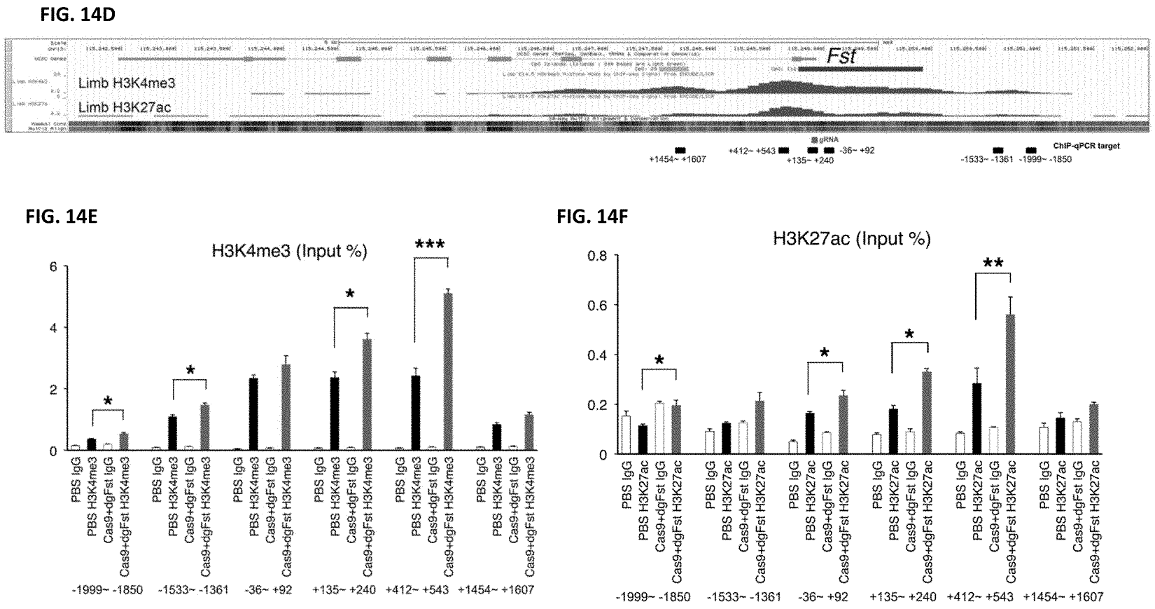

This example demonstrates amelioration of dystrophic phenotypes by a dual AAV-CRISPR/Cas9 TGA system that includes AAV-Cas9. To further demonstrate the potential therapeutic utility of CRISPR/Cas9 TGA, the AAV-dgRNA-MPH described herein was assayed in combination with a Cas9 AAV virus (AAV-SpCas9) for activation of target genes in vivo. Promoters and constructs were investigated, and AAV-CMVc-SpCas9 and AAV-nEF-SpCas9 (both driven by short, ubiquitous promoters of ˜500 bp) showed the best TGA efficiency. TGA efficiency was evaluated by co-injecting AAV-dgFst-T2-MPH with AAV-SpCas9 into the fore and hind limb muscles of wild-type mice at P2.5. At P21, the muscles were dissected, and levels of Fst expression were analyzed (FIG. 14A). Fst expression was elevated 9-fold, 28-fold, and 11-fold in the fore limb, TA, and QF muscles, respectively (compared with AAV-dgMock-MPH or no-injection controls) (FIG. 14B). Similar levels of Fst overexpression were observed when AAV-SpCas9 was replaced with nuclease-dead Cas9 (AAV-SpdCas9) (FIG. 14C). Using AAV-SpdCas9 minimizes DSBs for CRISPR/Cas9 TGA treatments in vivo. Co-injection of AAV-dgFst-MPH and AAV-SpCas9 also induced H3K4me3 and H3K27ac activation marks within Fst target sequences (FIGS. 7F and 14D-14F). Further, co-injection of AAV-SpCas9 with AAV-dgFst-MPH or AAV-dgUtrn-MPH ameliorated disease symptoms of mdx mice compared with AAV-dgMock controls (FIGS. 7A-7F). These data reveal that the dual-AAV in vivo CRISPR/Cas9 described herein can efficiently induce TGA to promote a therapeutic benefit.

### REFERENCES

1. Altucci, L., and Rots, M. G. (2016). Epigenetic drugs: from chemistry via biology to medicine and back. Clinical epigenetics 8, 56.
2. Burton, E. A., Tinsley, J. M., Holzfeind, P. J., Rodrigues, N. R., and Davies, K. E. (1999). A second promoter provides an alternative target for therapeutic up-regulation of utrophin in Duchenne muscular dystrophy. Proceedings of the National Academy of Sciences of the United States of America 96, 14025-14030.
3. Chavez, A., Scheiman, J., Vora, S., Pruitt, B. W., Tuttle, M., E, P. R. I., Lin, S., Kiani, S., Guzman, C. D., Wiegand, D. J., et al. (2015). Highly efficient Cas9-mediated transcriptional programming. Nature methods 12, 326-328.
4. Chavez, A., Tuttle, M., Pruitt, B. W., Ewen-Campen, B., Chari, R., Ter-Ovanesyan, D., Haque, S. J., Cecchi, R. J., Kowal, E. J., Buchthal, J., et al. (2016). Comparison of Cas9 activators in multiple species. Nature methods 13, 563-567.
5. Chen, M., and Qi, L. S. (2017). Repurposing CRISPR System for Transcriptional Activation. Advances in experimental medicine and biology 983, 147-157.
6. Chew, W. L., Tabebordbar, M., Cheng, J. K., Mali, P., Wu, E. Y., Ng, A. H., Zhu, K., Wagers, A. J., and Church, G. M. (2016). A multifunctional AAV-CRISPR-Cas9 and its host response. Nature methods 13, 868-874.
7. Czechanski, A., Byers, C., Greenstein, I., Schrode, N., Donahue, L. R., Hadjantonakis, A. K., and Reinholdt, L. G. (2014). Derivation and characterization of mouse embryonic stem cells from permissive and nonpermissive strains. Nature protocols 9, 559-574.
8. Dahlman, J. E., Abudayyeh, O. O., Joung, J., Gootenberg, J. S., Zhang, F., and Konermann, S. (2015). Orthogonal gene knockout and activation with a catalytically active Cas9 nuclease. Nature biotechnology 33, 1159-1161.
9. de Groote, M. L., Verschure, P. J., and Rots, M. G. (2012). Epigenetic Editing: targeted rewriting of epigenetic marks to modulate expression of selected target genes. Nucleic acids research 40, 10596-10613.
10. Esvelt, K. M., Mali, P., Braff, J. L., Moosburner, M., Yaung, S. J., and Church, G. M. (2013). Orthogonal Cas9 proteins for RNA-guided gene regulation and editing. Nature methods 10, 1116-1121.
11. Ferber, S., Halkin, A., Cohen, H., Ber, I., Einav, Y., Goldberg, I., Barshack, I., Seijffers, R., Kopolovic, J., Kaiser, N., et al. (2000). Pancreatic and duodenal homeobox gene 1 induces expression of insulin genes in liver and ameliorates streptozotocin-induced hyperglycemia. Nature medicine 6, 568-572.
12. Gilbert, L. A., Horlbeck, M. A., Adamson, B., Villalta, J. E., Chen, Y., Whitehead, E. H., Guimaraes, C., Panning, B., Ploegh, H. L., Bassik, M. C., et al. (2014). Genome-Scale CRISPR-Mediated Control of Gene Repression and Activation. Cell 159, 647-661.
13. Gilbert, L. A., Larson, M. H., Morsut, L., Liu, Z., Brar, G. A., Tones, S. E., Stern-Ginossar, N., Brandman, O., Whitehead, E. H., Doudna, J. A., et al. (2013). CRISPR-mediated modular RNA-guided regulation of transcription in eukaryotes. Cell 154, 442-451.
14. Gombash Lampe, S. E., Kaspar, B. K., and Foust, K. D. (2014). Intravenous injections in neonatal mice. Journal of visualized experiments: JoVE, e52037.
15. Haidet, A. M., Rizo, L., Handy, C., Umapathi, P., Eagle, A., Shilling, C., Boue, D., Martin, P. T., Sahenk, Z., Mendell, J. R., et al. (2008). Long-term enhancement of skeletal muscle mass and strength by single gene administration of myostatin inhibitors. Proceedings of the National Academy of Sciences of the United States of America 105, 4318-4322.
16. Hatanaka, F., Matsubara, C., Myung, J., Yoritaka, T., Kamimura, N., Tsutsumi, S., Kanai, A., Suzuki, Y., Sassone-Corsi, P., Aburatani, H., et al. (2010). Genome-wide profiling of the core clock protein BMAL1 targets reveals a strict relationship with metabolism. Molecular and cellular biology 30, 5636-5648.
17. Heerboth, S., Lapinska, K., Snyder, N., Leary, M., Rollinson, S., and Sarkar, S. (2014). Use of epigenetic drugs in disease: an overview. Genetics & epigenetics 6, 9-19.
18. Hunter, P. (2015). The second coming of epigenetic drugs: a more strategic and broader research framework could boost the development of new drugs to modify epigenetic factors and gene expression. EMBO reports 16, 276-279.
19. Imberti, B., Tomasoni, S., Ciampi, O., Pezzotta, A., Derosas, M., Xinaris, C., Rizzo, P., Papadimou, E., Novelli, R., Benigni, A., et al. (2015). Renal progenitors derived from human iPSCs engraft and restore function in a mouse model of acute kidney injury. Scientific reports 5, 8826.
20. Janghra, N., Morgan, J. E., Sewry, C. A., Wilson, F. X., Davies, K. E., Muntoni, F., and Tinsley, J. (2016). Correlation of Utrophin Levels with the Dystrophin Protein Complex and Muscle Fibre Regeneration in Duchenne and Becker Muscular Dystrophy Muscle Biopsies. PloS one 11, e0150818.
21. Jin, Y., Liu, R., Xie, J., Xiong, H., He, J. C., and Chen, N. (2013). Interleukin-10 deficiency aggravates kidney inflammation and fibrosis in the unilateral ureteral obstruction mouse model. Laboratory investigation; a journal of technical methods and pathology 93, 801-811.
22. Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
23. Jurkowski, T. P., Ravichandran, M., and Stepper, P. (2015). Synthetic epigenetics-towards intelligent control of epigenetic states and cell identity. Clinical epigenetics 7, 18.
24. Kearns, N. A., Genga, R. M., Enuameh, M. S., Garber, M., Wolfe, S. A., and Maehr, R. (2014). Cas9 effector-mediated regulation of transcription and differentiation in human pluripotent stem cells. Development 141, 219-223.
25. Kiani, S., Chavez, A., Tuttle, M., Hall, R. N., Chari, R., Ter-Ovanesyan, D., Qian, J., Pruitt, B. W., Beal, J., Vora, S., et al. (2015). Cas9 gRNA engineering for genome editing, activation and repression. Nature methods 12, 1051-1054.
26. Kim, J. Y., Grunke, S. D., Levites, Y., Golde, T. E., and Jankowsky, J. L. (2014). Intracerebroventricular viral injection of the neonatal mouse brain for persistent and widespread neuronal transduction. Journal of visualized experiments: JoVE, 51863.
27. Kobayashi, A., Valerius, M. T., Mugford, J. W., Carroll, T. J., Self, M., Oliver, G., and McMahon, A. P. (2008). Six2 defines and regulates a multipotent self-renewing nephron progenitor population throughout mammalian kidney development. Cell stem cell 3, 169-181.
28. Komor, A. C., Badran, A. H., and Liu, D. R. (2017). CRISPR-Based Technologies for the Manipulation of Eukaryotic Genomes. Cell 168, 20-36.
29. Konermann, S., Brigham, M. D., Trevino, A. E., Joung, J., Abudayyeh, O. O., Barcena, C., Hsu, P. D., Habib, N., Gootenberg, J. S., Nishimasu, H., et al. (2015). Genome-scale transcriptional activation by an engineered CRISPR-Cas9 complex. Nature 517, 583-588.
30. Kurosu, H., Yamamoto, M., Clark, J. D., Pastor, J. V., Nandi, A., Gurnani, P., McGuinness, O. P., Chikuda, H., Yamaguchi, M., Kawaguchi, H., et al. (2005). Suppression of aging in mice by the hormone Klotho. Science 309, 1829-1833.
31. La Russa, M. F., and Qi, L. S. (2015). The New State of the Art: Cas9 for Gene Activation and Repression. Molecular and cellular biology 35, 3800-3809.
32. Li, Z., Araoka, T., Wu, J., Liao, H. K., Li, M., Lazo, M., Zhou, B., Sui, Y., Wu, M. Z., Tamura, I., et al. (2016). 3D Culture Supports Long-Term Expansion of Mouse and Human Nephrogenic Progenitors. Cell stem cell 19, 516-529.
33. Liao, H. K., Gu, Y., Diaz, A., Marlett, J., Takahashi, Y., Li, M., Suzuki, K., Xu, R., Hishida, T., Chang, C. J., et al. (2015). Use of the CRISPR/Cas9 system as an intracellular defense against HIV-1 infection in human cells. Nature communications 6, 6413.
34. Ling, G. S., Cook, H. T., Botto, M., Lau, Y. L., and Huang, F. P. (2011). An essential protective role of IL-10 in the immunological mechanism underlying resistance vs. susceptibility to lupus induction by dendritic cells and dying cells. Rheumatology 50, 1773-1784.
35. Long, C., Amoasii, L., Mireault, A. A., McAnally, J. R., Li, H., Sanchez-Ortiz, E., Bhattacharyya, S., Shelton, J. M., Bassel-Duby, R., and Olson, E. N. (2016). Postnatal genome editing partially restores dystrophin expression in a mouse model of muscular dystrophy. Science 351, 400-403.
36. Nelson, C. E., Hakim, C. H., Ousterout, D. G., Thakore, P. I., Moreb, E. A., Castellanos Rivera, R. M., Madhavan, S., Pan, X., Ran, F. A., Yan, W. X., et al. (2016). In vivo genome editing improves muscle function in a mouse model of Duchenne muscular dystrophy. Science 351, 403-407.
37. Panesso, M. C., Shi, M., Cho, H. J., Paek, J., Ye, J., Moe, O. W., and Hu, M. C. (2014). Klotho has dual protective effects on cisplatin-induced acute kidney injury. Kidney international 85, 855-870.
38. Perez-Pinera, P., Kocak, D. D., Vockley, C. M., Adler, A. F., Kabadi, A. M., Polstein, L. R., Thakore, P. I., Glass, K. A., Ousterout, D. G., Leong, K. W., et al. (2013). RNA-guided gene activation by CRISPR-Cas9-based transcription factors. Nature methods 10, 973-976.
39. Pfister, S. X., and Ashworth, A. (2017). Marked for death: targeting epigenetic changes in cancer. Nature reviews Drug discovery 16, 241-263.
40. Platt, R. J., Chen, S., Zhou, Y., Yim, M. J., Swiech, L., Kempton, H. R., Dahlman, J. E., Parnas, O., Eisenhaure, T. M., Jovanovic, M., et al. (2014). CRISPR-Cas9 knockin mice for genome editing and cancer modeling. Cell 159, 440-455.
41. Qi, L. S., Larson, M. H., Gilbert, L. A., Doudna, J. A., Weissman, J. S., Arkin, A. P., and Lim, W. A. (2013). Repurposing CRISPR as an RNA-guided platform for sequence-specific control of gene expression. Cell 152, 1173-1183.
42. Rafael, J. A., Tinsley, J. M., Potter, A. C., Deconinck, A. E., and Davies, K. E. (1998). Skeletal muscle-specific expression of a utrophin transgene rescues utrophin-dystrophin deficient mice. Nature genetics 19, 79-82.
43. Sanjana, N. E., Cong, L., Zhou, Y., Cunniff, M. M., Feng, G., and Zhang, F. (2012). A transcription activator-like effector toolbox for genome engineering. Nature protocols 7, 171-192.
44. Schaefer, K. A., Wu, W. H., Colgan, D. F., Tsang, S. H., Bassuk, A. G., and Mahajan, V. B. (2017). Unexpected mutations after CRISPR-Cas9 editing in vivo. Nature methods 14, 547-548.
45. Sicinski, P., Geng, Y., Ryder-Cook, A. S., Barnard, E. A., Darlison, M. G., and Barnard, P. J. (1989). The molecular basis of muscular dystrophy in the mdx mouse: a point mutation. Science 244, 1578-1580.
46. Sienkiewicz, D., Kulak, W., Okurowska-Zawada, B., Paszko-Patej, G., and Kawnik, K. (2015). Duchenne muscular dystrophy: current cell therapies. Therapeutic advances in neurological disorders 8, 166-177.
47. Suzuki, K., Tsunekawa, Y., Hernandez-Benitez, R., Wu, J., Zhu, J., Kim, E. J., Hatanaka, F., Yamamoto, M., Araoka, T., Li, Z., et al. (2016). In vivo genome editing via CRISPR/Cas9 mediated homology-independent targeted integration. Nature 540, 144-149.
48. Swiech, L., Heidenreich, M., Banerjee, A., Habib, N., Li, Y., Trombetta, J., Sur, M., and Zhang, F. (2015). In vivo interrogation of gene function in the mammalian brain using CRISPR-Cas9. Nature biotechnology 33, 102-106.
49. Tabebordbar, M., Zhu, K., Cheng, J. K., Chew, W. L., Widrick, J. J., Yan, W. X., Maesner, C., Wu, E. Y., Xiao, R., Ran, F. A., et al. (2016). In vivo gene editing in dystrophic mouse muscle and muscle stem cells. Science 351, 407-411.
50. Takahashi, Y., Wu, J., Suzuki, K., Martinez-Redondo, P., Li, M., Liao, H. K., Wu, M. Z., Hernandez-Benitez, R., Hishida, T., Shokhirev, M. N., et al. (2017). Integration of CpG-free DNA induces de novo methylation of CpG islands in pluripotent stem cells. Science 356, 503-508.
51. Tanenbaum, M. E., Gilbert, L. A., Qi, L. S., Weissman, J. S., and Vale, R. D. (2014). A protein-tagging system for signal amplification in gene expression and fluorescence imaging. Cell 159, 635-646.
52. Tang, D. Q., Lu, S., Sun, Y. P., Rodrigues, E., Chou, W., Yang, C., Cao, L. Z., Chang, L. J., and Yang, L. J. (2006). Reprogramming liver-stem WB cells into functional insulin-producing cells by persistent expression of Pdx1- and Pdx1-VP16 mediated by lentiviral vectors. Laboratory investigation; a journal of technical methods and pathology 86, 83-93.
53. Thakore, P. I., Black, J. B., Hilton, I. B., and Gersbach, C. A. (2016). Editing the epigenome: technologies for programmable transcription and epigenetic modulation. Nature methods 13, 127-137.
54. Tinsley, J. M., Potter, A. C., Phelps, S. R., Fisher, R., Trickett, J. I., and Davies, K. E. (1996). Amelioration of the dystrophic phenotype of mdx mice using a truncated utrophin transgene. Nature 384, 349-353.
55. Vora, S., Tuttle, M., Cheng, J., and Church, G. (2016). Next stop for the CRISPR revolution: RNA-guided epigenetic regulators. The FEBS journal 283, 3181-3193.
56. Wehling-Henricks, M., Li, Z., Lindsey, C., Wang, Y., Welc, S. S., Ramos, J. N., Khanlou, N., Kuro, O. M., and Tidball, J. G. (2016). Klotho gene silencing promotes pathology in the mdx mouse model of Duchenne muscular dystrophy. Human molecular genetics 25, 2465-2482.
57. Zincarelli, C., Soltys, S., Rengo, G., and Rabinowitz, J. E. (2008). Analysis of AAV serotypes 1-9 mediated gene expression and tropism in mice after systemic injection. Molecular therapy: the journal of the American Society of Gene Therapy 16, 1073-1080.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

We claim:

1. A guide ribonucleic acid (gRNA), comprising from 5′ to 3′:
(a) a first region comprising a backbone sequence comprising at least 90% sequence identity to gttttagagcta (SEQ ID NO: 7) or guuuuagagcua (SEQ ID NO: 34);
a second region linked to a third region, wherein the second region comprises a modified MS2-binding loop sequence;
a third region linked to the second region, wherein the third region comprises a backbone sequence comprising at least 90% sequence identity to
(SEQ ID NO: 8)
tagcaagttaaaataaggctagtccgttatcaactt
or
(SEQ ID NO: 35)
uagcaaguuaaaauaaggcuaguccguuaucaacuu;
a fourth region linked to the third region, wherein the fourth region comprises the modified MS2-binding loop sequence; and
a fifth region linked to the fourth region, wherein the fifth region comprises a backbone sequence comprising at least 90% sequence identity to
(SEQ ID NO: 9)
aagtggcaccgagtcggtgctt
or
(SEQ ID NO: 36)
aaguggcaccgagucggugcuu;
wherein the modified MS2-binding loop sequence comprises at least two nucleotide changes to the native MS2-binding loop sequence ggccaacatgaggatcacccatgtctgcagggcc (SEQ ID NO: 12) or ggccaacaugaggaucacccaugucugcagggcc (SEQ ID NO: 39), wherein the at least two nucleotide changes increase the GC content and/or shorten repetitive content of the modified MS2-binding loop sequence relative to the native MS2-binding loop sequence; or
(b) a first region comprising a first modified backbone sequence;
a second region linked to the first region, wherein the second region comprises an MS2-binding loop sequence;
a third region linked to the second region, wherein the third region comprises a second modified backbone sequence;
a fourth region linked to the third region, wherein the fourth region comprises the MS2-binding loop sequence; and
a fifth region linked to the fourth region, wherein the fifth region comprises a backbone sequence comprising at least 90% sequence identity to
(SEQ ID NO: 9)
aagtggcaccgagtcggtgctt
or
(SEQ ID NO: 36)
aaguggcaccgagucggugcuu;
wherein the first modified backbone sequence comprises at least one nucleotide change to the native backbone sequence gttttagagcta (SEQ ID NO: 7) or guuuuagagcua (SEQ ID NO: 34),
wherein the second modified backbone sequence comprises at least one nucleotide change to the native backbone sequence tagcaagttaaaataaggctagtccgttatcaactt (SEQ ID NO: 8) or uagcaaguuaaaauaaggcuaguccguuaucaacuu (SEQ ID NO: 35), and
wherein each of the at least one nucleotide changes increase the GC content and/or shorten repetitive content of the first and second modified backbone sequences relative to the native backbone sequences.

2. The gRNA of claim 1, comprising a sixth region at the 5′-end of the gRNA and linked that is 14 to 30 nucleotides or ribonucleotides in length and comprises complementarity to a target nucleic acid molecule.

3. The gRNA of claim 2, wherein the target nucleic acid molecule is a gene whose decreased expression results in a disease or disorder in a mammal.

4. The gRNA of claim 1, wherein the wherein the first region comprises a U to C substitution and the third region comprises a A to G substitution.

5. The gRNA of claim 4, wherein
(a) the first region comprises or consists of the sequence gtttcagagcta (SEQ ID NO: 10) or guuucagagcua (SEQ ID NO: 37) and the third region comprises or consists of the sequence tagcaagttgaaataaggctagtccgttatcaactt (SEQ ID NO: 11) or uagcaaguugaaauaaggcuaguccguuaucaacuu (SEQ ID NO: 38);
(b) the modified MS2-binding loop sequence comprises or consists of the sequence tgctgaacatgaggatcacccatgtctgcagcagca (SEQ ID NO: 13), gggccaacatgaggatcacccatgtctgcagggccc (SEQ ID NO: 14), ggccagcatgaggatcacccatgcctgcagggcc (SEQ ID NO: 15), ugcugaacaugaggaucacccaugucugcagcagca (SEQ ID NO: 40), gggccaacaugaggaucacccaugucugcagggccc (SEQ ID NO: 41), or ggccagcaugaggaucacccaugccugcagggcc (SEQ ID NO: 42); or
(c) the gRNA comprises or consists of the sequence of SEQ ID NO: 1, SEQ ID NO: 2, SEQ ID NO: 3, SEQ ID NO: 4, SEQ ID NO: 28, SEQ ID NO: 29, SEQ ID NO: 30, or SEQ ID NO: 31.

6. A composition, comprising
the gRNA of claim 1, and
a pharmaceutically acceptable carrier.

7. A viral vector comprising the gRNA of claim 1.

8. A kit, comprising
the gRNA of claim 1;
a Cas9 protein, a dead Cas9 (dCas9) protein, a nucleic acid encoding a Cas9 protein, or a nucleic acid encoding a dCas9 protein, and
a MS2-transcriptional activator fusion protein or a nucleic acid encoding an MS2-transcriptional activator fusion protein.

9. A targeted gene activation (TGA) system, comprising:
(a) a first vector comprising a nucleic acid encoding a Cas9 or dCas9; and
(b) a second vector comprising the gRNA of claim 1 and a nucleic acid encoding an MS2-transcriptional activator fusion protein.

10. A method of increasing expression of at least one gene product in a subject, the method comprising:
administering a targeted gene activation (TGA) system to a subject, wherein the TGA system comprises:
a. a first vector comprising a nucleic acid encoding a Cas9 protein or dead Cas9 (dCas9) protein; and
b. a second vector comprising the gRNA of claim 1, and a nucleic acid encoding an MS2-transcriptional activator fusion protein;
wherein the TGA system infects a cell of the subject, thereby increasing expression of the at least one gene product in the infected cell.

11. The method of claim 10, wherein the first and second vector are adeno-associated viral (AAV) vectors.

12. The method of claim 10, wherein the least one gene product is encoded by an endogenous gene of the subject and the gRNA comprises a sixth region comprising complementarity to a sequence near the start site of the endogenous gene.

13. The method of claim 10, wherein the MS2-transcriptional activator fusion protein comprises an MS2 domain fused with a transcriptional activation domain comprising VP64, p65, MyoD1, HSF1, RTA, SET7/9, or any combination thereof.

14. The method of claim 13, wherein the MS2-transcriptional activator fusion protein comprises at least 90% sequence identity to SEQ ID NO: 18.

15. The method of claim 10, wherein the cell is in a muscle, liver, heart, lung, kidney, spinal cord, or stomach.

16. The method of claim 10, wherein the method comprises treating a disease associated with no or reduced expression of a gene by administering a therapeutically effective amount of the TGA system to the subject.

17. The method of claim 16, wherein the disease is type I diabetes, Duchenne muscular dystrophy, or acute kidney disease.

18. A vector system for measuring gene activation when Cas9 is expressed, comprising at least one gene activation vector and at least one reporter vector, wherein:
the at least one gene activation vector comprises a guide ribonucleic acid (gRNA) and at least one transcriptional activator protein; and
the at least one reporter vector comprises a target sequence of the gRNA and at least one reporter protein, wherein the reporter protein is positioned downstream of the target sequence.

19. A kit comprising the vector system of claim 18.

20. A method of measuring gene activation in a subject, comprising;
expressing Cas9 in the subject; and
injecting the subject with the at least one gene activation vector and the at least one reporter vector of the vector system of claim 18.

21. A gRNA or dgRNA comprising, in 5′ to 3′ order:
(i) a first backbone sequence;
(ii) a first MS2-binding loop sequence;
(iii) a second backbone sequence;
(iv) a second MS-2 binding loop sequence; and
(v) a third backbone sequence,
wherein at least one of (i)-(iv) are modified sequences that comprise one or more nucleotide changes that increase the GC content and/or shorten repetitive content relative to the corresponding native sequence.

22. One or more vectors comprising:
the gRNA or dgRNA of claim 21;
a Cas9 protein, a nucleic acid encoding a Cas9 protein, a dCas9 protein, or a nucleic acid encoding a dCas9 protein; and
a MS2-transcriptional activator fusion protein or a nucleic acid encoding a MS2-transcriptional activator fusion protein.

23. A method of increasing expression of at least one gene product of a cell, the method comprising contacting the cell with a vector comprising the gRNA or dgRNA of claim 21, wherein the cell comprises Cas9 or dCas9 protein and MS2-transcriptional activator fusion protein.

24. The method of claim 23, wherein the method further comprises contacting the cell with one or more vectors comprising:
a Cas9 protein, a nucleic acid encoding a Cas9 protein, a dCas9 protein, or a nucleic acid encoding a dCas9 protein; and
a MS2-transcriptional activator fusion protein or a nucleic acid encoding a MS2-transcriptional activator fusion protein.
